(19) United States
(12) Patent Application Publication
(10) Pub. No.: US 2020/0277268 A1
(43) Pub. Date: Sep. 3, 2020

(54) PENTAFLUOROPHENYL SULFONAMIDE COMPOUNDS, COMPOSITIONS AND USES THEREOF

(71) Applicants: Diana Sina, Mississauga (CA); DALRIADA THERAPEUTICS INC., Mississauga (CA)

(72) Inventors: Patrick Thomas Gunning, Mississauga (CA); Ji Sung Park, Milton (CA); Siawash Ahmar, Toronto (CA); David Alexander Rosa, Toronto (CA); Dziyana Kraskouskaya, Mississauga (CA); David Bakhshinyan, Richmond Hill (CA); Sheila Singh, Dundas (CA); Chitra Venugopal, Burlington (CA); Angelika Berger-Becvar, Mississauga (CA); Mulu Geletu-Heye, Mississauga (CA); Mariya Bogatchenko, Mississauga (CA); Elvin De Araujo, Mississauga (CA); Diana Sina, Mississauga (CA)

(73) Assignee: Dalriada Therapeutics Inc., Mississauga, ON (CA)

(21) Appl. No.: 16/649,806

(22) PCT Filed: Sep. 21, 2018
(86) PCT No.: PCT/CA2018/051191

§ 371 Date: Mar. 23, 2020

Related U.S. Application Data
(60) Provisional application No. 62/561,268, filed on Sep. 21, 2017.

(51) Int. Cl. C07D 295/135; C07C 311/16; C07C 311/20; C07D 213/42; C07D 241/04; C07D 307/52; C07D 209/14; A61P 35/00

(52) U.S. Cl. CPC C07D 295/135; C07C 311/16; C07C 311/20; A61P 35/00; C07D 241/04; C07D 307/52; C07D 209/14; C07D 213/42

(57) ABSTRACT

The present application relates to sulfonamide containing compounds of Formulae (I) and (II) and compositions containing said compounds effective in the treatment of cell proliferative disorders, in particular cancer, and various methods of use thereof.

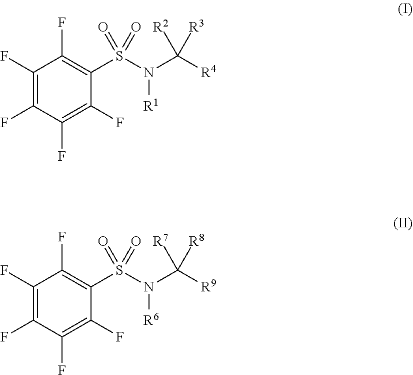

### RELATED APPLICATIONS

The present application claims the benefit of priority from U.S. provisional patent application Ser. No. 62/561,268 filed on Sep. 21, 2017, the contents of which are incorporated herein by reference in their entirety.

### FIELD

The present application relates to sulfonamide containing compounds and compositions containing said compounds effective in the treatment of cell proliferative disorders, in particular cancer, and various methods of use thereof.

### INTRODUCTION

Uncontrolled cell proliferation presents the underlying basis of many biological disorders. A prominent class of such disorders is various types of cancer. Despite the recent developments in cancer therapeutic agents such as DNA-alkylating agents, DNA intercalators, hormone analogs, and metabolite analogs, there is still need to develop therapeutic agents that selectively target malignant cells while leaving healthy cells intact and that present amenable pharmacokinetic profile with regard to availability, distribution, metabolism and toxicity.

Attachment of a small protein modifier called ubiquitin-fold modifier 1 (UFM1) to target proteins is a form of post-translational modification.¹ Attachment occurs through a three enzyme cascade, consisting of an E1-activating enzyme (UBA5), an E2-conjugating enzyme (UFC1) and an E3-ligase, that act in series.² The attachment of UFM1 to substrates, called UFMylation, has implications in numerous disease states. UFM1 enhances breast cancer progression when conjugated to components of the estrogen receptor system.³ Specifically, in invasive breast ductal carcinoma (MCF-7) UFM1 modifies ASC1 protein substrate which increases its affinity to ERα promoter regions, ultimately resulting in an upregulation of pro-proliferative genes, such as c-Myc, pS2 and Cyclin D1.³ UFM1 may also be responsible for the prevention of endoplasmic reticulum (ER) stress induced apoptosis.⁴ Furthermore, UFMylation plays a vital role in erythroid development and erythropoietin production.⁵ The Broad Institute's dependency map study, using RNAi and CRISPR loss-of-function screens against UBA5 showed that cell viability of the following listed cancers is dependent on UBA5 activity: leukemia, bile duct, fibroblast, kidney, mesothelioma, multiple myeloma, liver, central nervous system, soft tissue, pancreas, thyroid, gastric, ovary, upper aerodigestive tract, urinary tract, lung, skin, colorectal, esophagus, breast, uterus, cervix, bone, peripheral nervous system and lymphoma. In particular, acute myeloid leukemia (AML) cells, among other cancers, were shown to be highly dependent upon UBA5 E1 enzyme.^{6,7}

U.S. Pat. No. 6,482,860B1 discloses pentafluorophenylsulfonamide containing compounds for the treatment of cell proliferative diseases such as psoriasis and cancer.

### SUMMARY

The present application describes a novel class of compounds having strong anti-cancer activity. Strong cancer-killing potency (IC₅₀<5 μM) of exemplary compounds has been demonstrated in various cell cultures, such as major types of acute myeloid leukemia (AML), medulloblastoma (MB) and glioblastoma (GBM), including in patient-derived cells. In addition to strong anti-cancer activity, exemplary compounds of the application were found to meet and/or exceed other clinically desired parameters, including high metabolic stability.

Accordingly, in some embodiments, the present application includes a compound of Formula I or a pharmaceutically acceptable salt and/or solvate thereof:

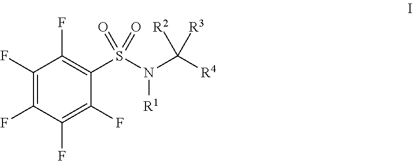

wherein:
R¹ is selected from C₁₋₁₀alkyl, C₂₋₁₀alkenyl, C₂₋₁₀alkynyl, C(O)C₁₋₁₀alkyl, C₃₋₁₀cycloalkyl, aryl, heterocycloalkyl, heteroaryl, CH₂C₃₋₁₀cycloalkyl, CH₂aryl, CH₂heterocycloalkyl and CH₂heteroaryl, the latter 8 of which are each optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆ alkenyl, OC₂₋₆ alkynyl, C₁₋₆ alkyleneOC₁₋₆ alkyl, C₁₋₆ alkyleneOC₂₋₆ alkenyl, C₁₋₆ alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl) and NHC(O)C₁₋₆alkyl;
R², and R³ are each independently selected from H, C₁₋₆ alkyl, C₂₋₆ alkenyl and C₂₋₆ alkynyl; or both R² and R³ combine to form ═O, or
R² and R³ together with the carbon to which they are attached form C₃₋₆cycloalkyl;
R⁴ is selected from aryl, heteroaryl, heterocycloalkyl, C₃₋₁₀cycloalkyl, C≡C-aryl, C≡C-heteroaryl, and C≡C-heterocycloalkyl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆ alkyl, OC₂₋₆ alkenyl, OC₂₋₆ alkynyl, C₁₋₆ alkyleneOC₁₋₆ alkyl, C₁₋₆ alkyleneOC₂₋₆ alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆ alkenyl, C(O)OC₂₋₆ alkynyl, S(O)ₓC₁₋₆ alkyl, S(O)ₓC₂₋₆ alkenyl, S(O)ₓC₂₋₆ alkynyl, C(O)NH₂, C(O)NHC₁₋₆alkyl, C(O)N(C₁₋₆alkyl(C₁₋₆alkyl), NHC(O)C₁₋₆alkyl and R⁵;
R⁵ is selected from Z—C₃₋₁₀cycloalkyl, Z-heterocycloalkyl, Z-aryl and Z-heteroaryl, each of which is optionally substituted with one or more of halo, CN, OH, NH₂, ═O, CO₂H, SO₂F, C₁₋₁₀alkyl, C₂₋₁₀ alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆ alkynyl, C(O)OC₁₋₆ alkyl, C(O)OC₂₋₆ alkenyl, C(O)OC₂₋₆ alkynyl, S(O)ₓC₁₋₆ alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl), NHC(O)C₁₋₆alkyl, C₃₋₁₀cycloalkyl, aryl, heteroaryl and heterocycloalkyl, the latter four groups being further optionally substituted by C₁₋₆ alkyl, C(O)C₁₋₆ alkyl and benzyl;
x is 0, 1 or 2;
Z is selected from a direct bond, C₁₋₄alkylene, O, NH, S, SO and SO₂ and all alkyl, alkenyl, alkynyl, aryl, heteroaryl, heterocycloalkyl and alkylene groups are optionally halosubstituted, provided that when R¹ is CH₂C₃₋₁₀cycloalkyl, the cycloalkyl group is not substituted with C(O)OC₁₋₆alkyl and when R¹ is cyclopropyl, R⁴ is not phenyl substituted with quinazoline.

In some embodiments, the present application includes a compound of Formula I or a pharmaceutically acceptable salt and/or solvate thereof:

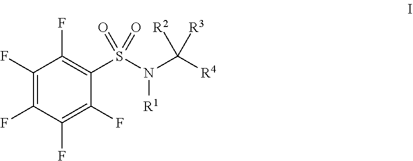

wherein:
R¹ is selected from C₃₋₁₀ alkyl, C₃₋₁₀ alkenyl, C₃₋₁₀ alkynyl, C(O)C₁₋₁₀alkyl, C₃₋₁₀ cycloalkyl, aryl, heterocycloalkyl, heteroaryl, CH₂C₃₋₁₀cycloalkyl, CH₂aryl and CH₂heterocycloalkyl, the latter 7 of which are each optionally substituted with one or more of halo, CN, OH, NH₂, ═O, CO₂H, SO₂F, C₁₋₁₀alkyl, C₂₋₁₀ alkenyl, C₂₋₁₀ alkynyl, NH(C₁₋₆ alkyl), N(C₁₋₆ alkyl)(C₁₋₆ alkyl), OC₁₋₆ alkyl, OC₂₋₆ alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl C(O)NHC₁₋₆alkyl, C(O)N(C₁₋₆alkyl(C₁₋₆alkyl) and NHC(O)C₁₋₆alkyl;
R², and R³ are each independently selected from H, C₁₋₆alkyl, C₂₋₆alkenyl and C₂₋₆alkynyl; or both R² and R³ combine to form ═O, or
R² and R³ together with the carbon to which they are attached form C₃₋₆cycloalkyl;
R⁴ is selected from aryl, heteroaryl, heterocycloalkyl and C₃₋₁₀cycloalkyl, each of which is optionally substituted with one or more of halo, CN, OH, NH, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl C(O)NHC₁₋₆alkyl, C(O)N(C₁₋₆alkyl(C₁₋₆alkyl), NHC(O)C₁₋₆alkyl and R⁵;
R⁵ is selected from Z—C₃₋₁₀cycloalkyl, Z-heterocycloalkyl, Z-aryl and Z-heteroaryl, each of which is optionally substituted with one or more of halo, CN, OH, NH₂, ═O, CO₂H, SO₂F, C₁₋₁₀alkyl, C₂₋₁₀alkenyl, C₂₋₁₀alkenyl, NH(C₁₋₆alkyl), N(C₁₋₆alkyl)(C₁₋₆alkyl), OC₁₋₆alkyl, OC₂₋₆alkenyl, OC₂₋alkynyl, C₁₋₆ alkyleneOC₁₋₆ alkyl, C₁₋₆ alkyleneOC₂₋₆ alkenyl, C₁₋₆ alkyleneOC₂₋₆ alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl C(O)NHC₁₋₆alkyl, C(O)N(C₁₋₆alkyl(C₁₋₆alkyl), NHC(O)C₁₋₆alkyl, C₃₋₁₀cycloalkyl, aryl, heteroaryl and heterocycloalkyl, the latter four groups being further optionally substituted by C₁₋₆alkyl, C(O)C₁₋₆alkyl and benzyl;
x is 0, 1 or 2;
Z is selected from a direct bond, C₁₋₄alkylene, O, NH, S, SO and SO₂ and
all alkyl, alkenyl, alkynyl, aryl, heteroaryl, heterocycloalkyl and alkylene groups are optionally halosubstituted, provided that when R¹ is CH₂C₃₋₁₀cycloalkyl, the cycloalkyl group is not substituted with C(O)OC₁₋₆ alkyl and when R¹ is cyclopropyl, R⁴ is not phenyl substituted with quinazoline.

In another aspect, the present application includes a composition comprising one or more compounds Formula I, and/or salts and/or solvates thereof, and one or more carriers. In some embodiments, the composition is a pharmaceutical composition and the one or more carriers are pharmaceutically acceptable.

In some embodiments, the present application includes a use of one or more compounds or compositions of the applications as a medicament.

In another aspect, the present application includes a method of treating a cell proliferative disorder comprising administering an effective amount of one or more of the compounds of this application to a subject in need thereof.

Other features and advantages of the present application will become apparent from the following detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The embodiments of the application will now be described in greater detail with reference to the attached drawings in which:

FIG. 1 shows glutathione-reactivity of (a) compound I-1 and (b) known microtubule inhibitor, Batabulin. Compound I-1 (100 μM, 40% DMSO) and Batabulin (100 μM, 1% DMSO) were incubated with 1-glutathione (10 mM) and consumption of test compounds was monitored by ¹⁹F-NMR spectroscopy.

FIG. 2 shows in panel (a) the clearance rate of exemplary compounds I-1 (square) and I-7 (diamond) in mouse hepatocytes. Panel (b) shows the clearance rate of a related literature compound (Batabulin).

FIG. 3 shows assessment of anti-microtubule activity of exemplary compound I-1. Negligible inhibition of tubulin polymerization by exemplary compound I-1 was observed in the assay probing for the change in optical density of the solution, as compared to the beta-tubulin inhibitor (Batabulin).

FIG. 4 shows assessment of competitive binding activity of exemplary compound I-1 against 132 kinases in a KINOMEscan™ platform (DiscoverX), summarized in a TREEspot™ interaction map. Exemplary compound I-1 (10 μM) showed negligible competitive binding towards 132 DNA-tagged kinases, which was measured via quantitative PCR of the DNA tag. Note false positive hit on mechanistic target of rapamycin (MTOR).

FIG. 5 shows assessment of competitive binding activity of exemplary compound I-1 in a BROMOscan™ platform (DiscoverX) against 32 bromodomains, summarized in a TREEspot™ interaction map. Exemplary compound I-1 (10 μM) showed negligible competitive binding towards 32 DNA-tagged bromodomains, which was measured via quantitative PCR of the DNA tag.

FIG. 6 shows ¹⁹F NMR assessment of covalent engagement of exemplary compound I-1 with cysteine-containing proteins. BSA (100 μM), lysozyme (100 μM) and STAT3/5 (12 and 15 μM, respectively) were incubated with exemplary compound I-1 (100 μM), and the generation of free fluoride ion (at −120 ppm) was monitored for covalent modification of the proteins and no significant fluoride release was observed.

FIG. 7 shows 1D ¹⁹F NMR spectra of 100 μM exemplary compound I-1 in the presence of 100 μM UBA5 at 25° C. following incubation for two hours at 37° C. in buffer (100 mM HEPES, pH 7.4, 100 μM 5-fluoro-Trp, with a final concentration of 10% D₂O and 10% DMSO). Spectra were normalized and referenced according to the fluorine peak of 5-fluoro-Trp. Fluoride was released in the presence of UBA5.

FIG. 8 shows MS analysis of exemplary compound I-1 and UBA5, showing covalent adduct formation at 45, 571 Da.

FIG. 9 shows Western blot analysis after dosing of MV4-11 cells with exemplary compound I-1, at an 8 hour time-point. Blots probed with antibodies against: i. UBA5, ii. UFC1, UFM1, iv. c-Myc and v. β-actin loading control. Concentrations tested ranged from 0 to 1 μM as indicated.

FIG. 10 shows Western blot analysis after exemplary compound I-55 (a) and I-40 (b) dosing of MV4-11 cells, at 8 hour time-points. Blots probed with antibodies against: i. UBA5, ii. UFC1, and iii. β-actin loading control. Concentrations tested ranged from 0 to 1 μM as indicated.

FIG. 11 shows transthiolation assay of exemplary compounds. Levels of UFM1-UFC1 conjugate formation is monitored for UBA5 inhibition. % Inhibition values result from quantifying UFM1-UFC1 conjugate intensity of reactions with 50 μM or 10 μM test compound against normal reaction (NR) control.

FIG. 12 shows transthiolation assay of I-1, using reduced levels of UBA5 protein (50 nM). Levels of UFM1-UFC1 conjugate formation is monitored for UBA5 inhibition as compared for normal reaction (NR) control. Concentrations of I-1 tested ranges from 0 μM to 1 μM.

FIG. 13 shows thermal shift assay results showing negative derivative plot of UBA5 with and without 50 μM I-1.

FIG. 14 A. shows DFT calculated TS1 for reaction of T138067 with CH₃S⁻ nucleophile. B. Calculated Reaction Profile of T138067 with CH₃S— Nucleophile.

FIG. 15 shows effects of select compounds were tested on cell proliferation in patient derived GBM BTIC lines: GBM8 and BT428. Concentrations tested were: 62.5 nM, 125 nM, 250 nM and no compound control.

### DESCRIPTION OF VARIOUS EMBODIMENTS

### I. Definitions

Unless otherwise indicated, the definitions and embodiments described in this and other sections are intended to be applicable to all embodiments and aspects of the present application herein described for which they are suitable as would be understood by a person skilled in the art.

The term “compound of the application” or “compound of the present application” and the like as used herein refers to a compound Formula I or II, and pharmaceutically acceptable salts and/or solvates thereof.

The term “composition of the application” or “composition of the present application” and the like as used herein refers to a composition, such a pharmaceutical composition, comprising one or more compounds of the application.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “a compound” should be understood to present certain aspects with one compound or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “agent” as used herein indicates a compound or mixture of compounds that, when added to a composition, tend to produce a particular effect on the composition's properties.

The term “and/or” as used herein means that the listed items are present, or used, individually or in combination. In effect, this term means that “at least one of” or “one or more” of the listed items is used or present.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown or named in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

The present application refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “haloalkyl” as used herein refers to an alkyl group wherein one or more, including all of the hydrogen atoms are replaced by a halogen atom.

The term “halosubstituted” as used herein refers to a chemical group wherein one or more, including all of the hydrogen atoms, are replaced by a halogen atom.

The term “cycloalkyl,” as used herein, whether it is used alone or as part of another group, means a saturated carbocyclic group containing a number of carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms. When a cycloalkyl group contains more than one ring, the rings may be fused, bridged, spirofused or linked by a bond.

The term “aryl” as used herein, whether it is used alone or as part of another group, refers to cyclic groups containing from 6 to 10 carbon atoms and one or more rings, at least one of which is aromatic ring. When an aryl group contains more than one ring, the rings may be fused, bridged, spirofused or linked by a bond. In some embodiments of the application, the aryl group contains from 6, 9 or 10 carbon atoms, such as phenyl, indanyl or naphthyl.

The term “heterocycloalkyl” as used herein, whether it is used alone or as part of another group, refers to cyclic groups containing 3 to 10 atoms, and at least one non-aromatic ring in which one or more of the atoms are a heteromoiety selected from O, S, S(O), SO₂, N, NH and NC₁₋₆alkyl. Heterocycloalkyl groups are either saturated or unsaturated (i.e. contain one or more double bonds) and contain one or more than one ring (i.e. are polycyclic). When a heterocycloalkyl group contains more than one ring, the rings may be fused, bridged, spirofused or linked by a bond. When a heterocycloalkyl group contains the prefix Cₙ₁₋ₙ₂ this prefix indicates the number of carbon atoms in the corresponding carbocyclic group in which one or more of the ring atoms is replaced with a heteromoiety as defined above.

A first ring group being “fused” with a second ring group means the first ring and the second ring share at least two atoms there between.

The term “heteroaryl” as used herein refers to cyclic groups containing from 5 to 10 atoms, one or more rings, at least one of which is aromatic ring, and at least one heteromoiety selected from O, S, S(O), SO₂, N, NH and NC₁₋₆alkyl. When a heteroaryl group contains more than one ring, the rings may be fused, bridged, spirofused or linked by a bond. When a heteroaryl group contains the prefix Cₙ₁₋ₙ₂ this prefix indicates the number of carbon atoms in the corresponding carbocyclic group in which one or more of the ring atoms is replaced with a heteromoiety as defined above.

The term “available”, as in “available hydrogen atoms” or “available atoms” refers to atoms that would be known to a person skilled in the art to be capable of replacement by a substituent.

The terms “halo” or “halogen” as used herein, whether it is used alone or as part of another group, refers to a halogen atom and includes fluoro, chloro, bromo and iodo.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3rd Edition, 1999 and in Kocienski, P. Protecting Groups, 3rd Edition, 2003, Georg Thieme Verlag (The Americas).

The term “subject” as used herein includes all members of the animal kingdom including mammals, and suitably refers to humans. Thus the methods of the present application are applicable to both human therapy and veterinary applications

The term “pharmaceutically acceptable” means compatible with the treatment of a subject.

The term “pharmaceutically acceptable carrier” means a non-toxic solvent, dispersant, excipient, adjuvant and/or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of a subject.

The term “solvate” as used herein means a compound, or a salt or prodrug of a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

As used herein, the term “effective amount” or “therapeutically effective amount” means an amount of one or more compounds or compositions of the application that is effective, at dosages and for periods of time necessary to achieve the desired result. For example in the context of treating a cell proliferative disorder, an effective amount is an amount that, for example, decreases said cell proliferation compared to the inhibition without administration of the one or more compounds or compositions. In an embodiment, effective amounts vary according to factors such as the disease state, age, sex and/or weight of the subject. In a further embodiment, the amount of a given compound or composition that will correspond to an effective amount will vary depending upon factors, such as the given compound(s), the pharmaceutical formulation, the route of administration, the type of condition, disease or disorder, the identity of the subject being treated, and the like, but can nevertheless be routinely determined by one skilled in the art.

The term “administered” as used herein means administration of a therapeutically effective amount of one or more compounds or compositions of the application to a cell, tissue, organ or subject.

The term “cell proliferative disorder” as used herein refers to a disease, disorder or condition characterized by cells that have the capacity for autonomous growth or replication, e.g., an abnormal state or condition characterized by proliferative cell growth.

The term “neoplasm” as used herein refers to a mass of tissue resulting from the abnormal growth and/or division of cells in a subject having a cell proliferative disorder. Neoplasms can be benign (such as uterine fibroids and melanocytic nevi), potentially malignant (such as carcinoma in situ) or malignant (i.e. cancer). Exemplary cell proliferative disorders or neoplactic disorders include but are not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers.

The term “hematological malignancy” as used herein refers to cancers that affect blood and bone marrow.

The term “leukemia” as used herein means any disease involving the progressive proliferation of abnormal leukocytes found in hemopoietic tissues, other organs and usually in the blood in increased numbers. For example, leukemia includes acute myeloid leukemia, acute lymphocytic leukemia and chronic myeloma leukemia (CML) in blast crisis.

The term “lymphoma” as used herein means any disease involving the progressive proliferation of abnormal lymphoid cells. For example, lymphoma includes Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas. Non-Hodgkin's lymphomas can also for example be as classified using the WHO and REAL classification.

The term “myeloma” and/or “multiple myeloma” as used herein means any tumor or cancer composed of cells derived from the hemopoietic tissues of the bone marrow. Multiple myeloma is also knows as MM and/or plasma cell myeloma.

The term “glioblastoma” as used herein are malignant Grade IV brain tumors, where a large portion of tumor cells are reproducing and dividing at any given time. Glioblastomas are generally found in the cerebral hemispheres of the brain, but can be found anywhere in the brain.

### II. Compounds and Compositions of the Application

In one aspect, the present application includes a compound of Formula I or a pharmaceutically acceptable salt and/or solvate thereof:

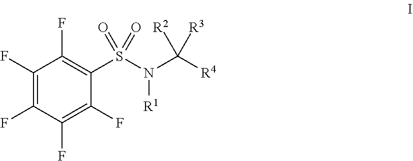

wherein:
R¹ is selected from C₁₋₁₀alkyl, C₂₋₁₀alkenyl, C₂₋₁₀alkynyl, C(O)C₁₋₁₀alkyl, C₃₋₁₀cycloalkyl, aryl, heterocycloalkyl, heteroaryl, CH₂C₃₋₁₀cycloalkyl, CH₂aryl, CH₂heterocycloalkyl and CH₂heteroaryl, the latter 8 of which are each optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆ alkyl), N(C₁₋₆ alkyl)(C₁₋₆ alkyl), OC₁₋₆ alkyl, OC₂₋₆ alkenyl, OC₂₋₆ alkynyl, C₁₋₆ alkyleneOC₁₋₆ alkyl, C₁₋₆ alkyleneOC₂₋₆ alkenyl, C₁₋₆ alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆ alkynyl, S(O)ₓC₁₋₆ alkyl, S(O)ₓC₂₋₆ alkenyl, S(O)ₓC₂₋₆ alkynyl, C(O)NH₂, C(O)NHC₁₋₆ alkyl, C(O)N(C₁₋₆alkyl(C₁₋₆alkyl) and NHC(O)C₁₋₆alkyl;
R², and R³ are each independently selected from H, C₁₋₆alkyl, C₂₋₆alkenyl and C₂₋₆alkynyl; or both R² and R³ combine to form ═O, or
R² and R³ together with the carbon to which they are attached form C₃₋₆cycloalkyl;
R⁴ is selected from aryl, heteroaryl, heterocycloalkyl, C₃₋₁₀cycloalkyl, C≡C-aryl, C≡C-heteroaryl, and C≡C-heterocycloalkyl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl), NHC(O)C₁₋₆alkyl and R⁵;
R⁵ is selected from Z—C₃₋₁₀ cycloalkyl, Z-heterocycloalkyl, Z-aryl and Z-heteroaryl, each of which is optionally substituted with one or more of halo, CN, OH, NH₂, ═O, CO₂H, SO₂F, C₁₋₁₀alkyl, C₂₋₁₀alkenyl, C₂₋₁₀ alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl), NHC(O)C₁₋₆alkyl, C₃₋₁₀cycloalkyl, aryl, heteroaryl and heterocycloalkyl, the latter four groups being further optionally substituted by C₁₋₆alkyl, C(O)C₁₋₆alkyl and benzyl;
x is 0, 1 or 2;
Z is selected from a direct bond, C₁₋₄ alkylene, O, NH, S, SO and SO₂ and
all alkyl, alkenyl, alkynyl, aryl, heteroaryl, heterocycloalkyl and alkylene groups are optionally halosubstituted, provided that when R¹ is CH₂C₃₋₁₀cycloalkyl, the cycloalkyl group is not substituted with C(O)OC₁₋₆alkyl and when R¹ is cyclopropyl, R⁴ is not phenyl substituted with quinazoline.

In another aspect, the present application includes a compound of Formula I or a pharmaceutically acceptable salt and/or solvate thereof:

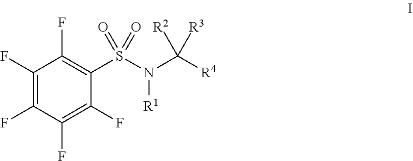

wherein:
R¹ is selected from C₃₋₁₀alkyl, C₃₋₁₀alkenyl, C₃₋₁₀alkynyl, C(O)C₁₋₁₀alkyl, C₃₋₁₀cycloalkyl, aryl, heterocycloalkyl, heteroaryl, CH₂C₃₋₁₀cycloalkyl, CH₂aryl and CH₂heterocycloalkyl, the latter 7 of which are each optionally substituted with one or more of halo, CN, OH, NH, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆ alkenyl, C(O)C₂₋₆ alkynyl, C(O)OC₁₋₆ alkyl, C(O)OC₂₋₆ alkenyl, C(O)OC₂₋₆ alkynyl, S(O)ₓC₁₋₆alkyl, S(O)ₓC₂₋₆alkenyl, S(O)ₓC₂₋₆alkynyl C(O)NHC₁₋₆alkyl, C(O)N(C₁₋₆alkyl(C₁₋₆alkyl) and NHC(O)C₁₋₆alkyl;
R², and R³ are each independently selected from H, C₁₋₆ alkyl, C₂₋₆ alkenyl and C₂₋₆ alkynyl; or both R² and R³ combine to form ═O, or
R² and R³ together with the carbon to which they are attached form C₃₋₆cycloalkyl;
R⁴ is selected from aryl, heteroaryl, heterocycloalkyl and C₃₋₁₀cycloalkyl, each of which is optionally substituted with one or more of halo, CN, OH, NH₂, ═O, CO₂H, SO₂F, C₁₋₁₀alkyl, C₂₋₁₀alkenyl, C₂₋₁₀ alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl C(O)NHC₁₋₆alkyl, C(O)N(C₁₋₆alkyl(C₁₋₆alkyl), NHC(O)C₁₋₆alkyl and R⁵;
R⁵ is selected from Z—C₃₋₁₀cycloalkyl, Z-heterocycloalkyl, Z-aryl and Z-heteroaryl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆ alkyleneOC₁₋₆ alkyl, C₁₋₆ alkyleneOC₂₋₆ alkenyl, C₁₋₆ alkyleneOC₂₋₆ alkynyl, C(O)C₁₋₆ alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl, C(O)NHC₁₋₆alkyl, C(O)N(C₁₋₆alkyl(C₁₋₆alkyl), NHC(O)C₁₋₆alkyl, C₃₋₁₀ cycloalkyl, aryl, heteroaryl and heterocycloalkyl, the latter four groups being further optionally substituted by C₁₋₆alkyl, C(O)C₁₋₆alkyl and benzyl;
x is 0, 1 or 2;
Z is selected from a direct bond, C₁₋₄alkylene, O, NH, S, SO and SO₂ and
all alkyl, alkenyl, alkynyl, aryl, heteroaryl, heterocycloalkyl and alkylene groups are optionally halosubstituted, provided that when R¹ is CH₂C₃₋₁₀cycloalkyl, the cycloalkyl group is not substituted with C(O)OC₁₋₆ alkyl and when R¹ is cyclopropyl, R⁴ is not phenyl substituted with quinazoline.

Heterocycloalkyl includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran, tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine, homopiperazine, 1,3-dioxepane, 4,7-dihydro-1,3-dioxepin, and hexamethylene oxide.

Heteroaryl includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole. Additionally, heteroaryl encompasses polycyclic aromatic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In some embodiments, R¹ is CH₂C₃₋₁₀cycloalkyl optionally substituted with one to two of halo, CN, OH, NH₂, ═O, CO₂H, SO₂F, C₁₋₆alkyl, C₂₋₆alkenyl, C₂₋₆alkenyl, NH(C₁₋₄alkyl), N(C₁₋₄alkyl)(C₁₋₄alkyl), OC₁₋₄alkyl, OC₂₋₄alkenyl, OC₂₋₄alkynyl, C₁₋₄alkyleneOC₁₋₄alkyl, C₁₋₄alkyleneOC₂₋₄alkenyl, C₁₋₄ alkyleneOC₂₋₄ alkynyl, C(O)C₁₋₄ alkyl, C(O)C₂₋₄ alkenyl, C(O)C₂₋₄ alkynyl, C(O)OC₁₋₄alkyl, C(O)OC₂₋₄alkenyl, C(O)OC₂₋₄alkynyl, S(O)ₓC₁₋₄alkyl, S(O)ₓC₂₋₄alkenyl, S(O)ₓC₂₋₄alkynyl C(O)NHC₁₋₄alkyl, C(O)N(C₁₋₄alkyl(C₁₋₄alkyl) and NHC(O)C₁₋₄alkyl). In some embodiments, R¹ is unsubstituted CH₂cyclopropyl, CH₂cyclobutyl, CH₂cyclopentyl or CH₂cyclohexyl. In some embodiments, R¹ is unsubstituted CH₂cyclopropyl or CH₂cyclopropyl substituted with one substituent selected from C₁₋₂ alkyleneOC₁₋₄ alkyl, C₁₋₂ alkyleneOC₂₋₄ alkynyl, C(O)C₁₋₄ alkyl, C(O)C₂₋₄alkynyl, C(O)OC₁₋₄alkyl, and C(O)OC₂₋₄alkynyl.

In some embodiments, R¹ is CH₂heteroaryl, optionally substituted with one to two of halo, CN, OH, NH₂, ═O, CO₂H, SO₂F, C₁₋₆alkyl, C₂₋₆alkenyl, C₂₋₆alkenyl, NH(C₁₋₄alkyl), N(C₁₋₄alkyl)(C₁₋₄ alkyl), OC₁₋₄ alkyl, OC₂₋₄ alkenyl, OC₂₋₄ alkynyl, C₁₋₄ alkyleneOC₁₋₄ alkyl, C₁₋₄ alkyleneOC₂₋₄alkenyl, C₁₋₄alkyleneOC₂₋₄alkynyl, C(O)C₁₋₄alkyl, C(O)C₂₋₄alkenyl, C(O)C₂₋₄alkynyl, C(O)OC₁₋₄alkyl, C(O)OC₂₋₄ alkenyl, C(O)OC₂₋₄ alkynyl, S(O)ₓC₁₋₄ alkyl, S(O)ₓC₂₋₄ alkenyl, S(O)ₓC₂₋₄ alkynyl C(O)NHC₁₋₄alkyl, C(O)N(C₁₋₄alkyl(C₁₋₄alkyl) and NHC(O)C₁₋₄alkyl). In some embodiments, R¹ is unsubstituted CH₂pyridine, CH₂pyrazine, CH₂pyrimidine, CH₂pyridazine, CH₂thiophene, CH₂furan, CH₂pyrrole, CH₂imidazole, CH₂thiazole, CH₂oxazole, CH₂pyrazole, CH₂isothiazole or CH₂isoxazole. In some embodiments, R¹ is unsubstituted CH₂pyridine.

In some embodiments, R¹ is selected from C₃₋₁₀cycloalkyl and heterocycloalkyl, each of which is optionally substituted with one to two of halo, CN, OH, NH₂, ═O, C₁₋₆alkyl, C₂₋₆alkenyl, C₂₋₆ alkenyl, NH(C₁₋₄ alkyl), N(C₁₋₄ alkyl)(C₁₋₄ alkyl), OC₁₋₄ alkyl, OC₂₋₄ alkenyl, OC₂₋₄ alkynyl, C₁₋₄alkyleneOC₁₋₄alkyl, C₁₋₄alkyleneOC₂₋₄alkenyl, C₁₋₄alkyleneOC₂₋₄alkynyl, C(O)C₁₋₄alkyl, C(O)C₂₋₄alkenyl, C(O)C₂₋₄ alkynyl, C(O)OC₁₋₄ alkyl, C(O)OC₂₋₄ alkenyl, C(O)OC₂₋₄ alkynyl, S(O)ₓC₁₋₄ alkyl, S(O)ₓC₂₋₄alkenyl, S(O)ₓC₂₋₄alkynyl C(O)NHC₁₋₄alkyl, C(O)N(C₁₋₄alkyl(C₁₋₄alkyl) and NHC(O)C₁₋₄alkyl).

In some embodiments, R¹ is a C₃₋₆cycloalkyl optionally substituted with one or two substituents independently selected from OC₁₋₄ alkyl, C₁₋₄ alkyleneOC₁₋₄ alkyl, C₁₋₄ alkyleneOC₂₋₄alkynyl, C(O)C₁₋₄ alkyl, C(O)C₂₋₄ alkynyl, C(O)OC₁₋₄ alkyl, and C(O)OC₂₋₄ alkynyl. In some embodiments, R¹ is unsubstituted cyclopropyl, cyclobutyl, cyclopentyl or cyclohexyl. In some embodiments, R¹ is unsubstituted cyclopropyl or cyclopropyl substituted with one substituent selected from C₁₋₂alkyleneOC₁₋₄alkyl, C₁₋₂alkyleneOC₂₋₄alkynyl, C(O)C₁₋₄alkyl, C(O)C₂₋₄alkynyl, C(O)OC₁₋₄ alkyl, and C(O)OC₂₋₄ alkynyl.

In some embodiments, R¹ is selected from heterocycloalkyl, aryl and heteroaryl, each of which is optionally substituted with one or more of C₁₋₄ alkyl, C₂₋₄alkenyl, C₂₋₄ alkynyl halo, OH, ═O, NH₂, NH(C₁₋₄ alkyl), N(C₁₋₄ alkyl)(C₁₋₄ alkyl), OC₁₋₆ alkyl, OC₂₋₆ alkenyl, OC₂₋₆ alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₁₋₆alkynyl, C(O)OC₁₋₆alkyl, C(O)OC₂₋₆alkenyl, C(O)OC₂₋₆alkynyl, C(O)NHC₁₋₆alkyl and C(O)N(C₁₋₆ alkyl(C₁₋₆ alkyl). In some embodiments, R¹ is selected from furanyl, indolinyl, 1,2,3,4-tetrahydroquinolinyl and 1,2,3,4-tetrahydroisoquinolinyl attached through the nitrogen in R¹. In some embodiments, R¹ is unsubstituted oxetane or tetrahydrofuran, or oxetane or tetrahydrofuran substituted with one or more of two of halo, CN, OH, NH₂, ═O, C₁₋₆ alkyl, C₂₋₆alkenyl, C₂₋₆alkenyl, NH(C₁₋₄alkyl), N(C₁₋₄alkyl)(C₁₋₄alkyl), OC₁₋₄alkyl, OC₂₋₄alkenyl, OC₂₋₄alkynyl, C₁₋₄ alkyleneOC₁₋₄ alkyl, C₁₋₄ alkyleneOC₂₋₄ alkenyl, C₁₋₄ alkyleneOC₂₋₄ alkynyl, C(O)C₁₋₄ alkyl, C(O)C₂₋₄alkenyl, C(O)C₂₋₄alkynyl, C(O)OC₁₋₄alkyl, C(O)OC₂₋₄alkenyl, C(O)OC₂₋₄alkynyl, S(O)ₓC₁₋₄alkyl, S(O)ₓC₂₋₄alkenyl, S(O)ₓC₂₋₄alkynyl C(O)NHC₁₋₄alkyl, C(O)N(C₁₋₄alkyl(C₁₋₄alkyl) and NHC(O)C₁₋₄alkyl).

In some embodiments, R¹ is selected from C₁₋₁₀alkyl, C₂₋₁₀alkenyl, and C₂₋₁₀alkynyl. In some embodiments, R¹ is unsubstituted C₁₋₁₀alkyl. In some embodiments, R¹ is methyl or ethyl.

In some embodiments, R¹ is selected from

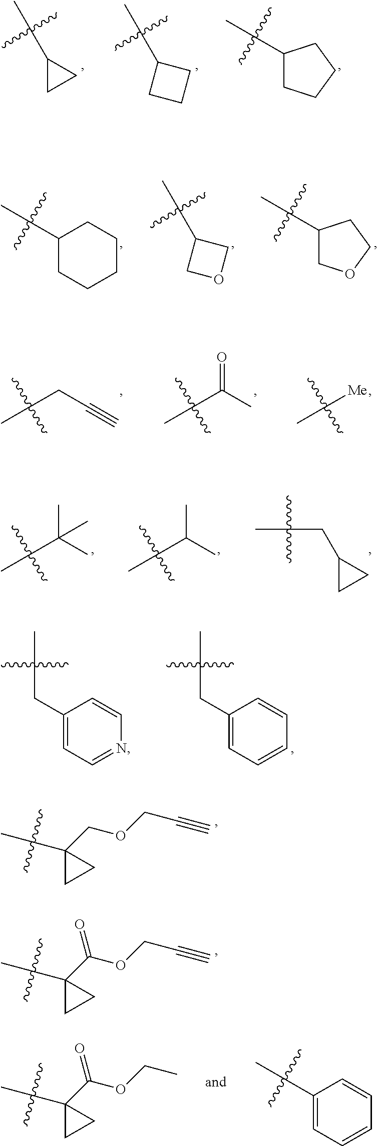

wherein the wavy line represents the point of attachment to the rest of the structure of Formula I.

In some embodiments, R¹ is selected from:

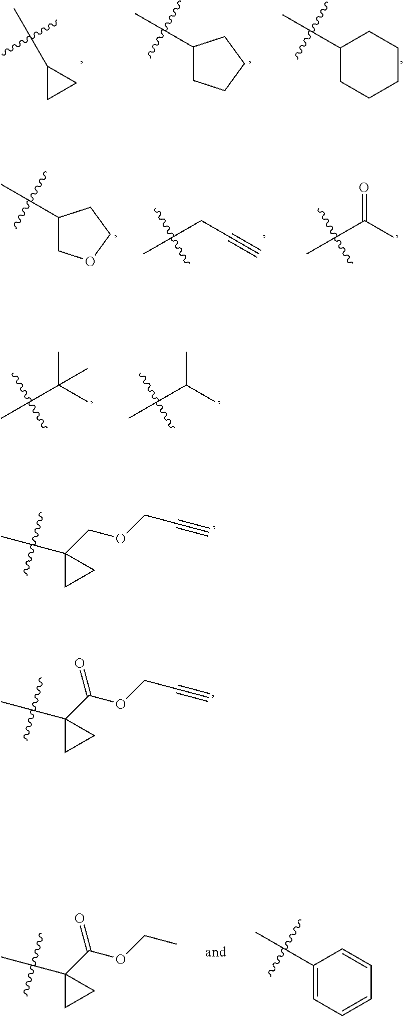

wherein the wavy line represents the point of attachment to the rest of the structure of Formula I.

In some embodiments, R² and R³ are each independently selected from H, C₁₋₆alkyl, C₁₋₆fluoroalkyl and C₃₋₁₀cycloalkyl. In some embodiments, at least one of R² and R³ is H. In some embodiments, both R² and R³ are H.

In some embodiments, both R² and R³ combine to form ═O.

In some embodiments, both R² and R³ together with the carbon to which they are attached form C₃₋₆cycloalkyl. In some embodiments, both R² and R³ together with the carbon to which they are attached form cyclopentyl, In some embodiments, R⁴ is independently selected from phenyl, pyridinyl, quinazolinyl, quinolinyl, indanyl, pyrazolyl, isooxazole, quinazoline and pyrrolo[2,3-b]pyridinyl optionally substituted with one, two or three F, Br, Cl, CF₃, CF₃O, CO₂H, CN, CONH₂, CO₂C₁₋₆ alkyl C₃₋₆ cycloalkyl, C₃₋₆ heterocycloalkyl, C₁₋₄ alkyl, OC₁₋₄ alkyl, C₁₋₄ alkynyl, OC₁₋₄alkynyl, NH₂, NHC₁₋₄alkyl N(C₁₋₄alkyl)₂, NHC(O)C₁₋₄alkyl, SO₂C₁₋₄alkyl, phenyl and heteroaryl, wherein the phenyl, heteroaryl, cycloalkyl and heterocycloalkyl groups are independently further optionally substituted with one, two or three F, Br, Cl, CF₃, CF₃O, CO₂H, CN, CONH₂, CO₂C₁₋₆alkyl C₃₋₆cycloalkyl, C₃₋₆heterocycloalkyl, C₁₋₄alkyl, OC₁₋₄alkyl, C₁₋₄alkynyl, OC₁₋₄alkynyl, NH₂, NHC₁₋₄alkyl N(C₁₋₄alkyl)₂, NHC(O)C₁₋₄alkyl, and SO₂C₁₋₄alkyl.

In some embodiments, R⁴ is independently selected from phenyl, pyridinyl, quinazolinyl, quinolinyl, indanyl, pyrazolyl, isooxazole, quinazoline and pyrrolo[2,3-b]pyridinyl optionally substituted with one, two or three F, Cl, CF₃O, CO₂H, CN, C₃₋₆cycloalkyl, C₁₋₄alkyl, OC₁₋₄alkyl, C₁₋₄ alkynyl, OC₁₋₄ alkynyl, NH₂, NHC₁₋₄ alkyl N(C₁₋₄ alkyl)₂, NHC(O)C₁₋₄ alkyl, SO₂C₁₋₄ alkyl, phenyl and heteroaryl, wherein the phenyl and heteroaryl groups are further optionally substituted with one, two or three F, Br, Cl, CF₃, CF₃O, CO₂H, CN, CONH₂, CO₂C₁₋₆alkyl C₃₋₆cycloalkyl, C₃₋₆heterocycloalkyl, C₁₋₄alkyl, OC₁₋₄alkyl, C₁₋₄alkynyl, OC₁₋₄alkynyl, NH₂, NHC₁₋₄alkyl N(C₁₋₄alkyl)₂, NHC(O)C₁₋₄alkyl, and SO₂C₁₋₄alkyl.

In some embodiments, R⁴ is selected from C≡C-aryl, C≡C-heteroaryl, and C≡C-heterocycloalkyl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl C(O)NHC₁₋₆alkyl, C(O)N(C₁₋₆alkyl(C₁₋₆alkyl), and NHC(O)C₁₋₆alkyl. In some embodiments, R⁴ is selected from C≡C-aryl wherein aryl is unsubstituted phenyl or phenyl substituted with one, two or three F, Br, Cl, CF₃, CF₃O, CO₂H, CN, CONH₂, CO₂C₁₋₆ alkyl C₃₋₆ cycloalkyl, C₃₋₆ heterocycloalkyl, C₁₋₄ alkyl, OC₁₋₄alkyl, C₁₋₄alkynyl, OC₁₋₄alkynyl, NH₂, NHC₁₋₄alkyl N(C₁₋₄alkyl)₂, NHC(O)C₁₋₄alkyl, and SO₂C₁₋₄alkyl.

In some embodiments, R⁴ is selected from:

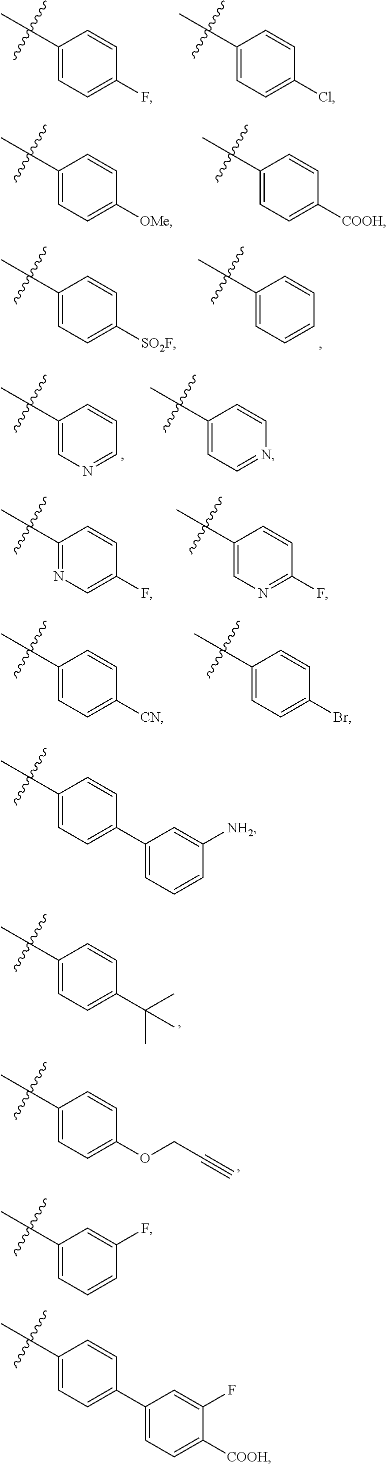

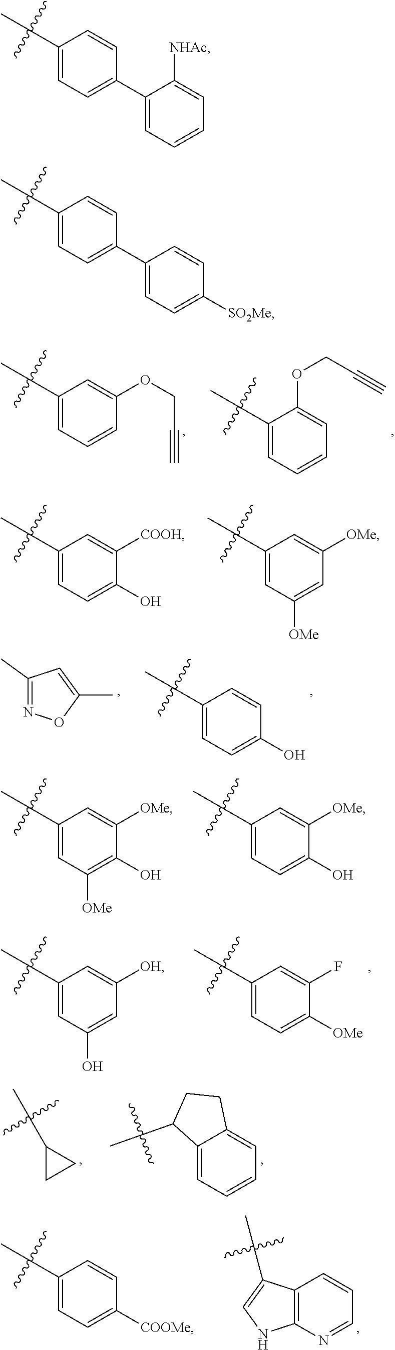

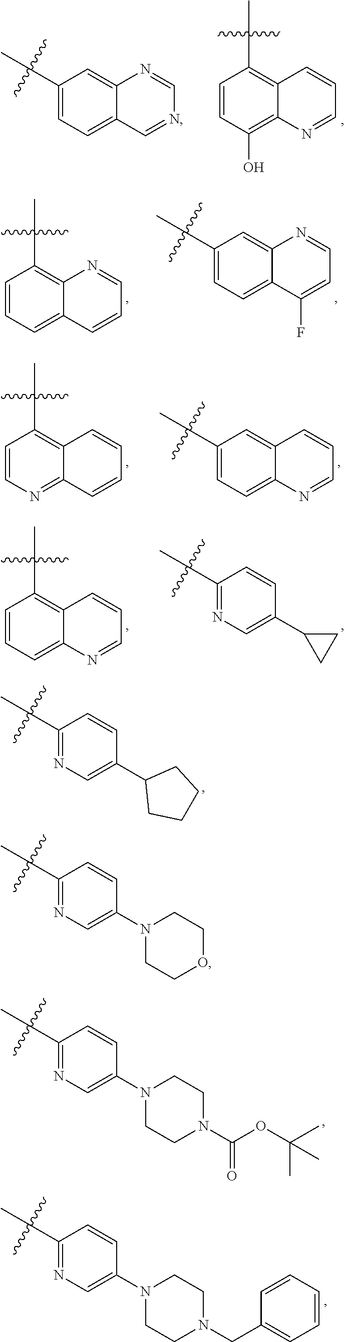

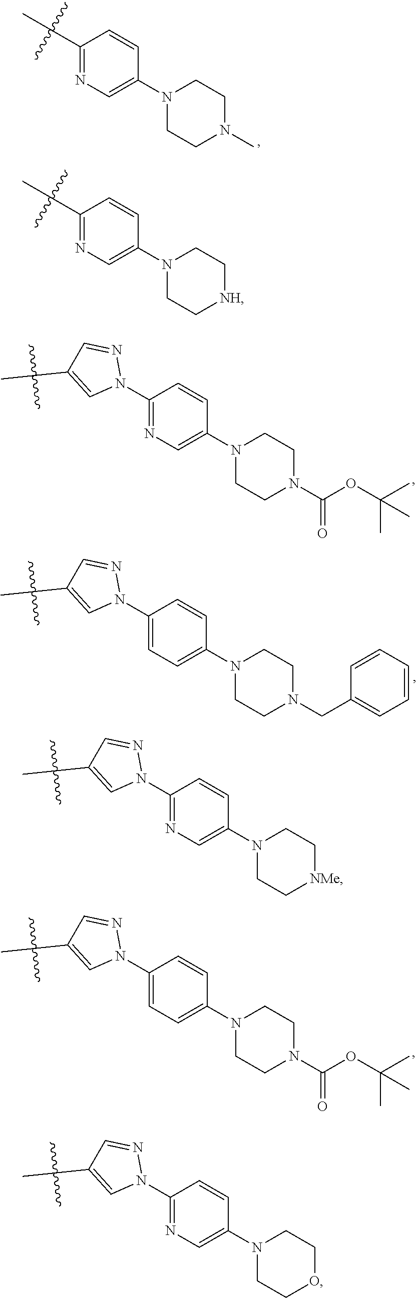

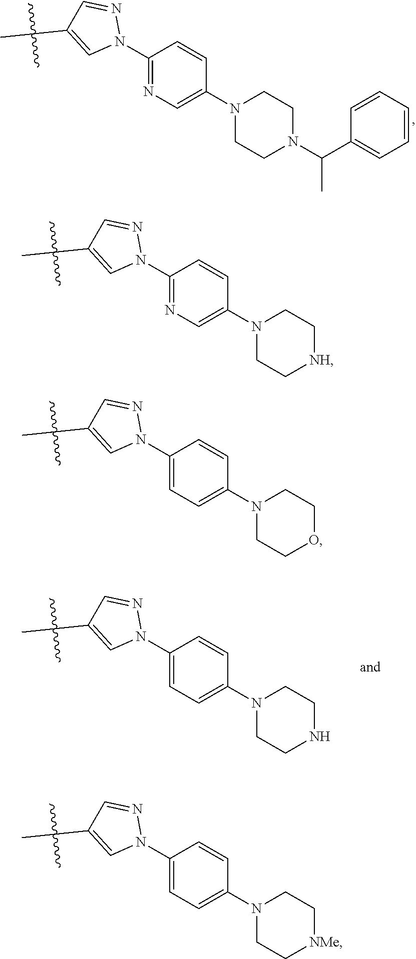 [SMILES: *c1cnn(-c2ccc(N3CCN(C(C)c4ccccc4)CC3)cn2)c1 ; *c1cnn(-c2ccc(N3CCNCC3)cn2)c1 ; *c1cnn(-c2ccc(N3CCOCC3)cc2)c1 ; *c1cnn(-c2ccc(N3CCNCC3)cc2)c1 ; *c1cnn(-c2ccc(N3CCN(C)CC3)cc2)c1]

where the wavy line represents the point of attachment to the rest of the structure of Formula I.

In some embodiments, R⁴ is selected from:

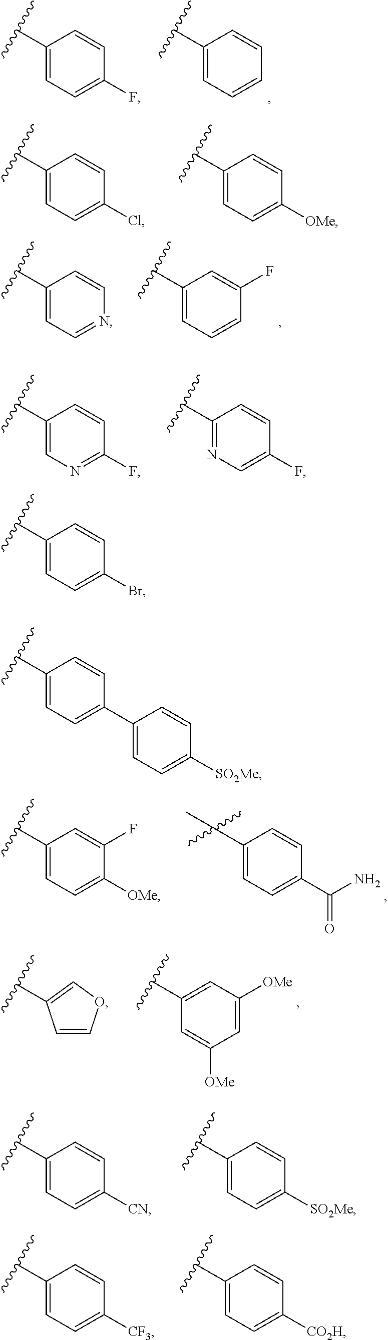

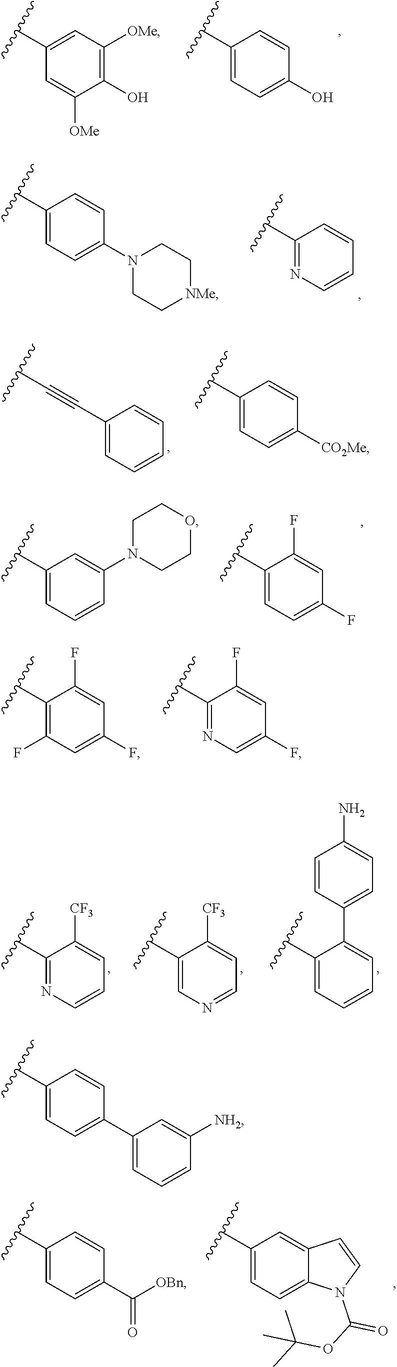

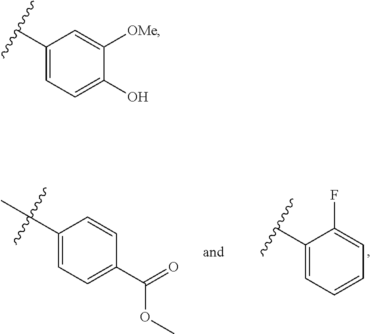 [SMILES: *c1ccc(O)c(OC)c1 ; *c1ccc(C(=O)OC)cc1 ; *c1ccccc1F]

where the wavy line represents the point of attachment to the rest of the structure of Formula I.

In some embodiments, the compound of Formula I is selected from:

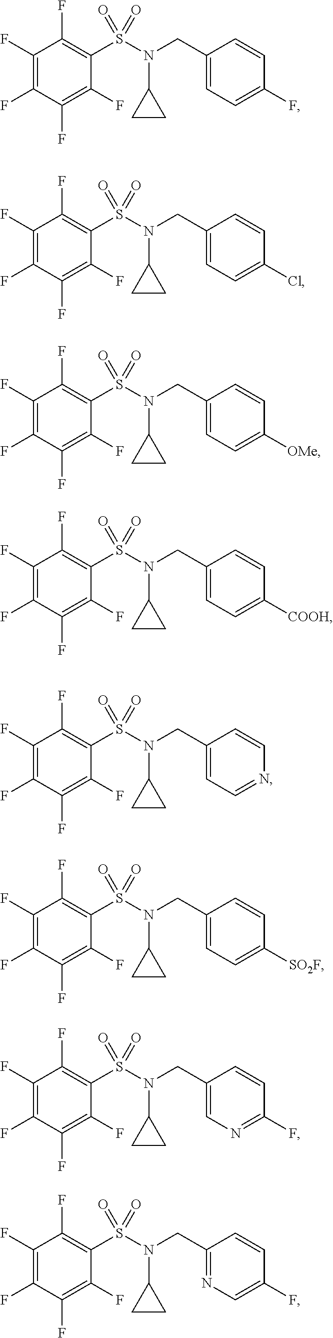

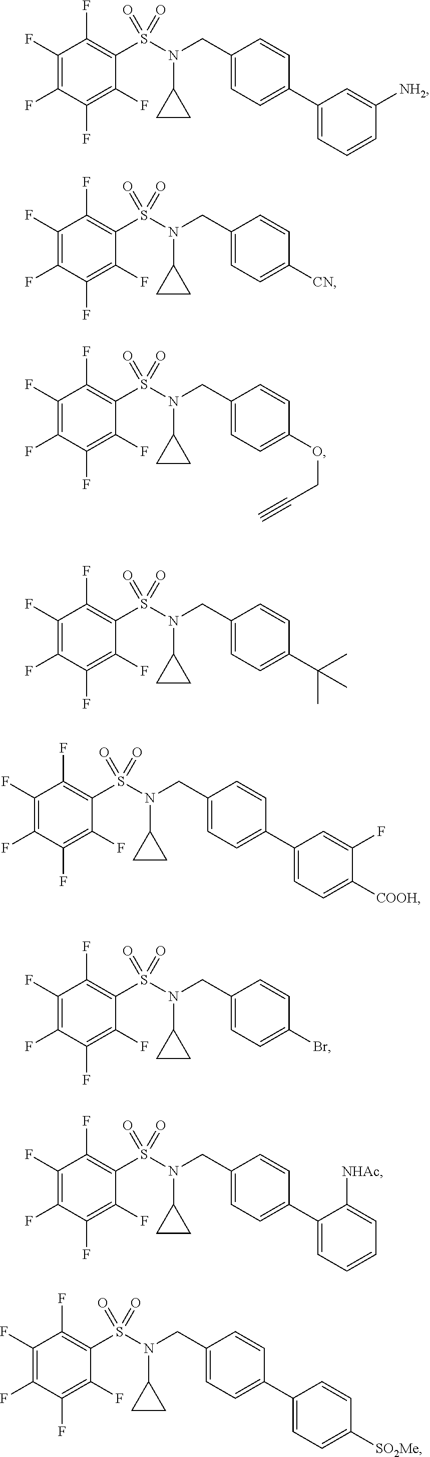

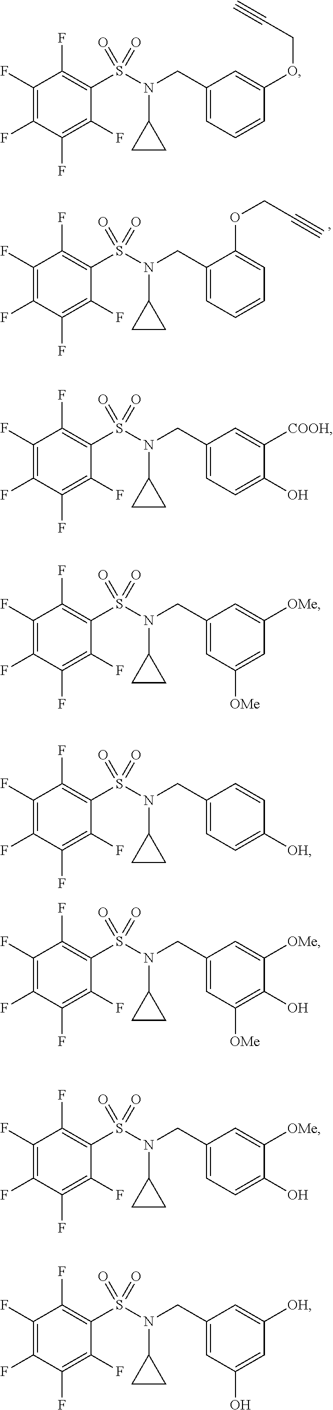

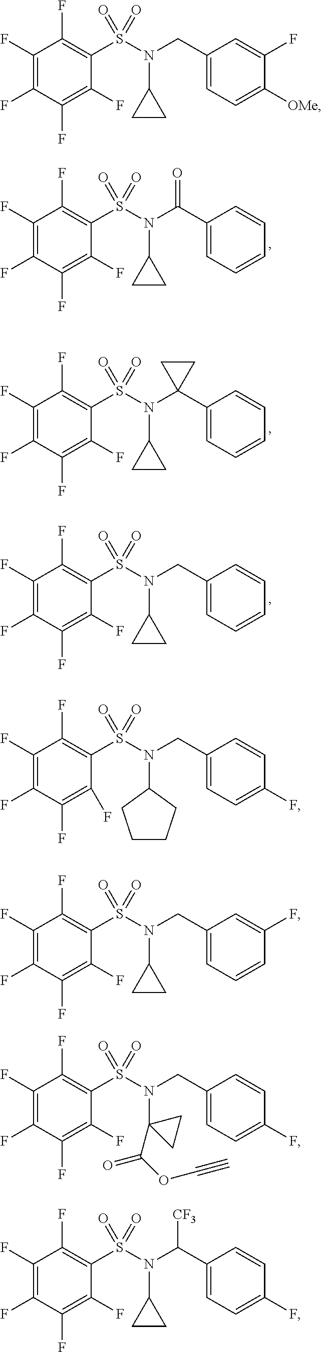

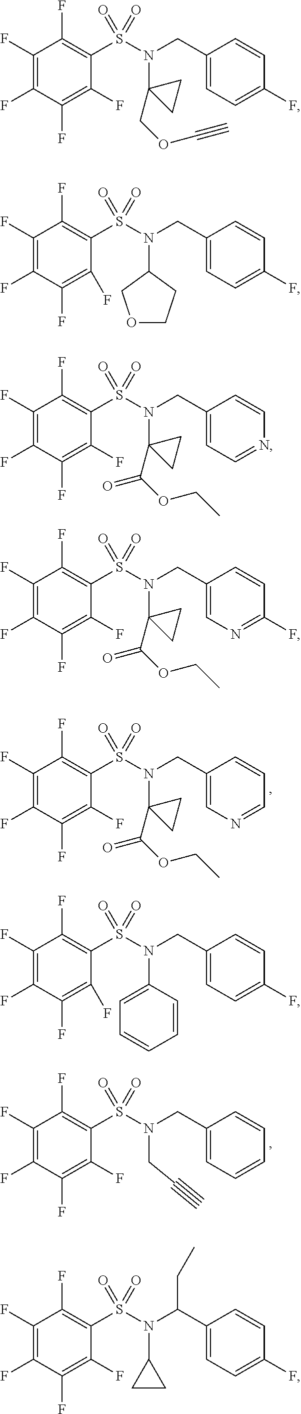

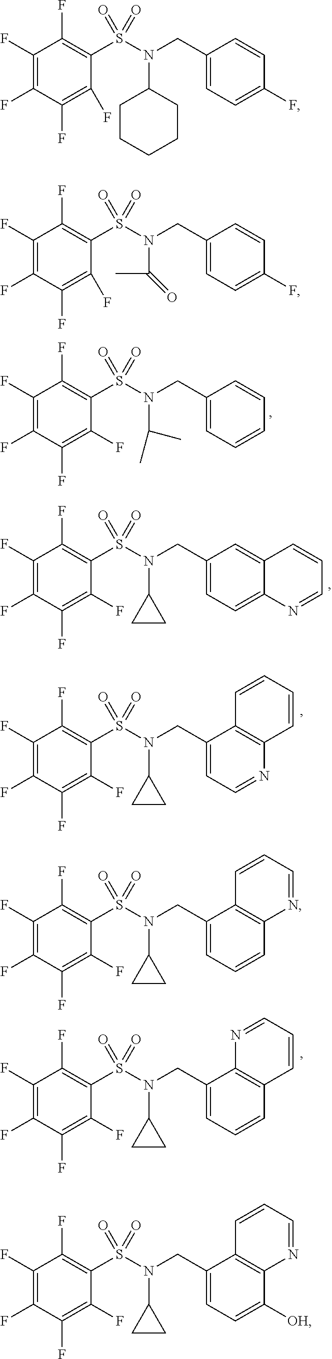

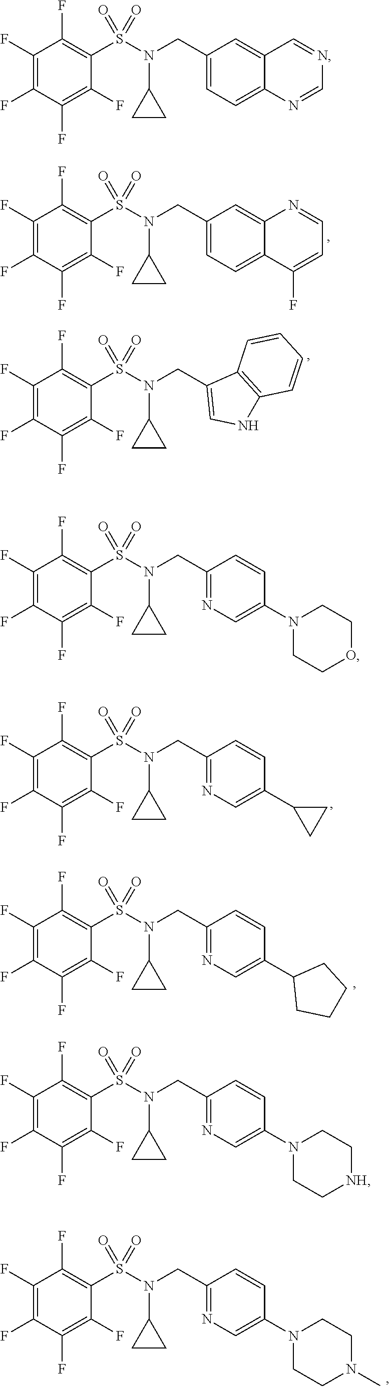

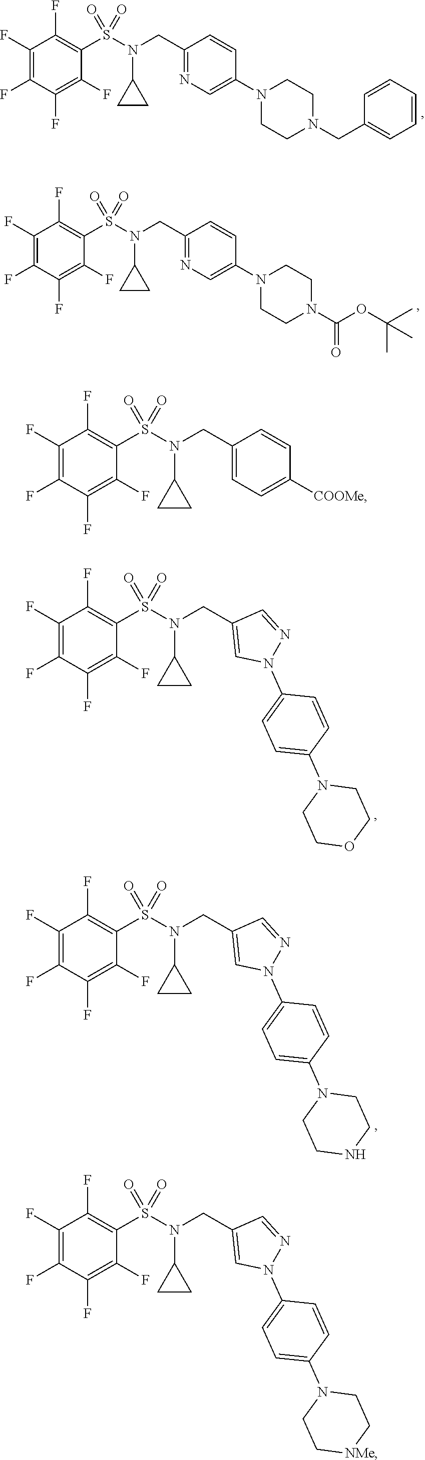

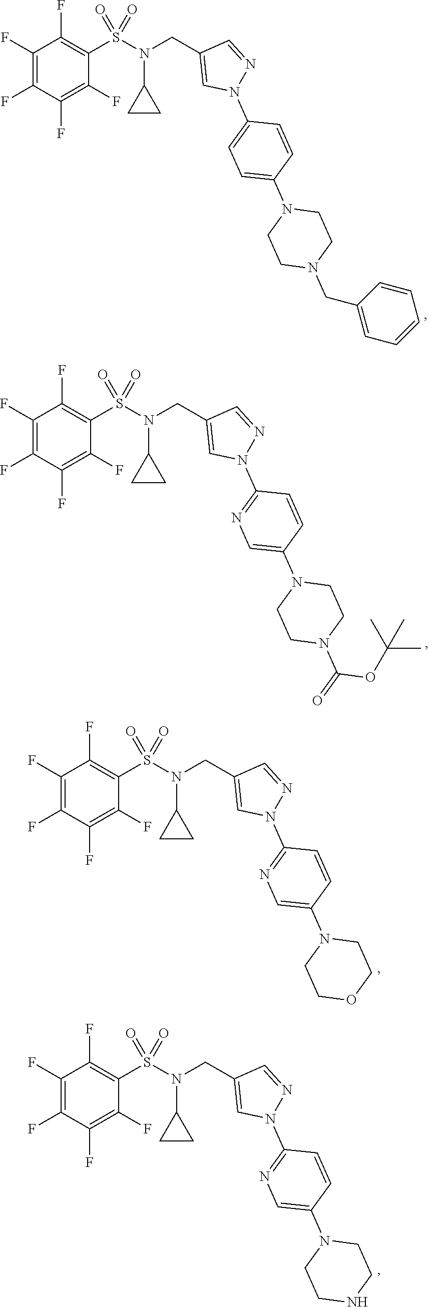 [SMILES: O=S(=O)(c1c(F)c(F)c(F)c(F)c1F)N(Cc1cnn(-c2ccc(N3CCN(Cc4ccccc4)CC3)cc2)c1)C1CC1 ; CC(C)(C)OC(=O)N1CCN(c2ccc(-n3cc(CN(C4CC4)S(=O)(=O)c4c(F)c(F)c(F)c(F)c4F)cn3)nc2)CC1 ; O=S(=O)(c1c(F)c(F)c(F)c(F)c1F)N(Cc1cnn(-c2ccc(N3CCOCC3)cn2)c1)C1CC1 ; O=S(=O)(c1c(F)c(F)c(F)c(F)c1F)N(Cc1cnn(-c2ccc(N3CCNCC3)cn2)c1)C1CC1]

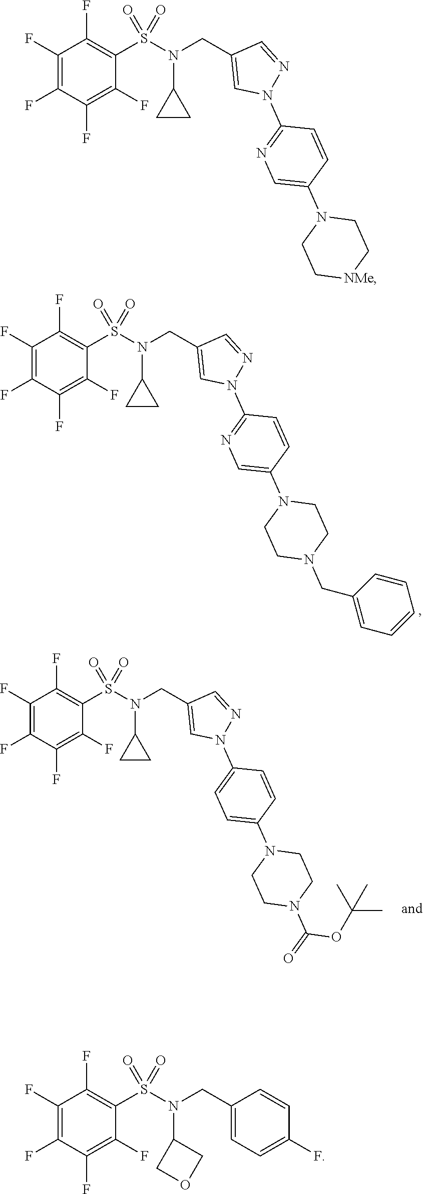 [SMILES: CN1CCN(c2ccc(-n3cc(CN(C4CC4)S(=O)(=O)c4c(F)c(F)c(F)c(F)c4F)cn3)nc2)CC1 ; O=S(=O)(c1c(F)c(F)c(F)c(F)c1F)N(Cc1cnn(-c2ccc(N3CCN(Cc4ccccc4)CC3)cn2)c1)C1CC1 ; CC(C)(C)OC(=O)N1CCN(c2ccc(-n3cc(CN(C4CC4)S(=O)(=O)c4c(F)c(F)c(F)c(F)c4F)cn3)cc2)CC1 ; O=S(=O)(c1c(F)c(F)c(F)c(F)c1F)N(Cc1ccc(F)cc1)C1COC1]

In some embodiments, the compound of Formula I is selected from:

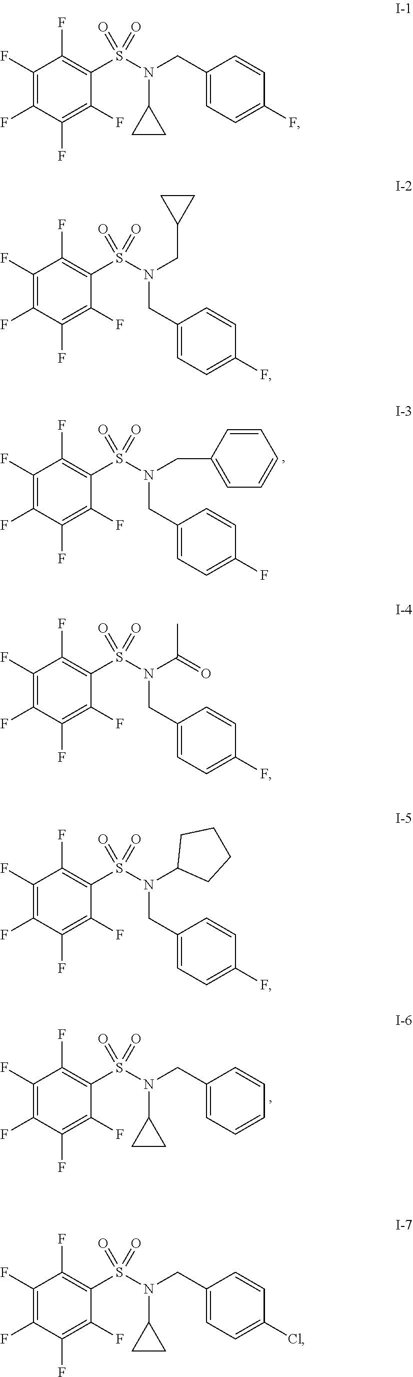

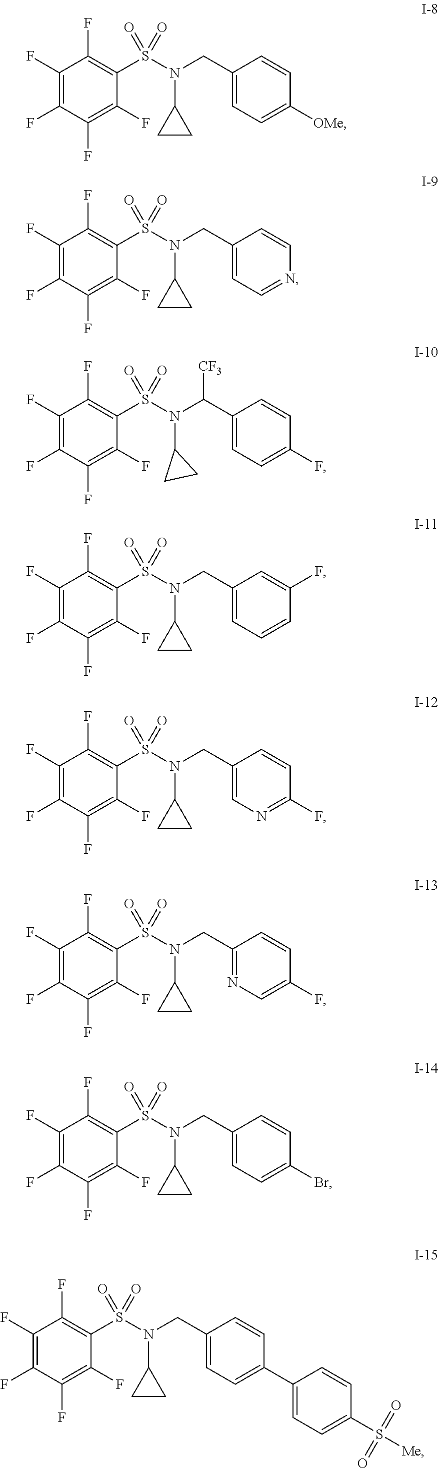

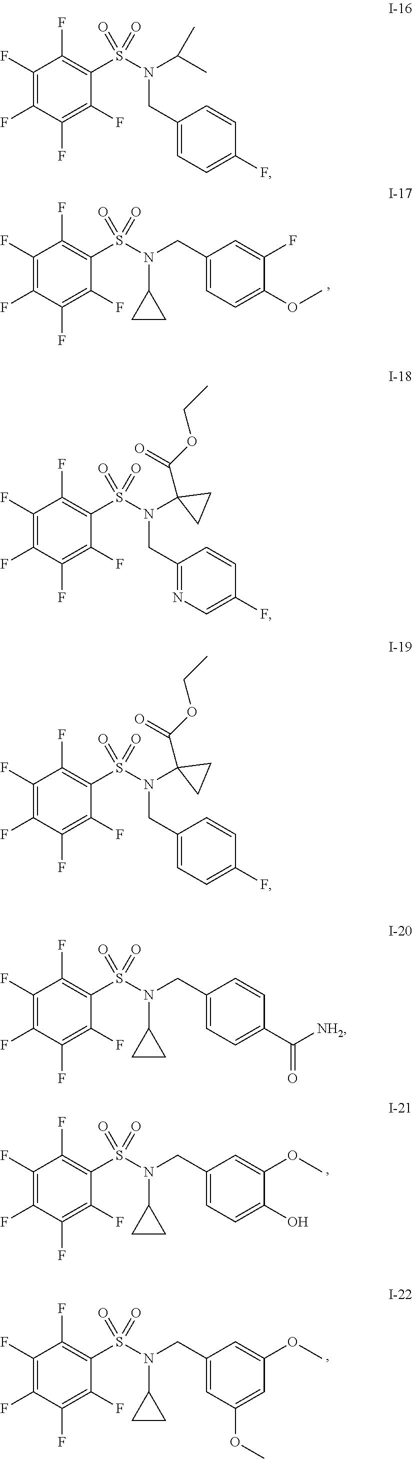

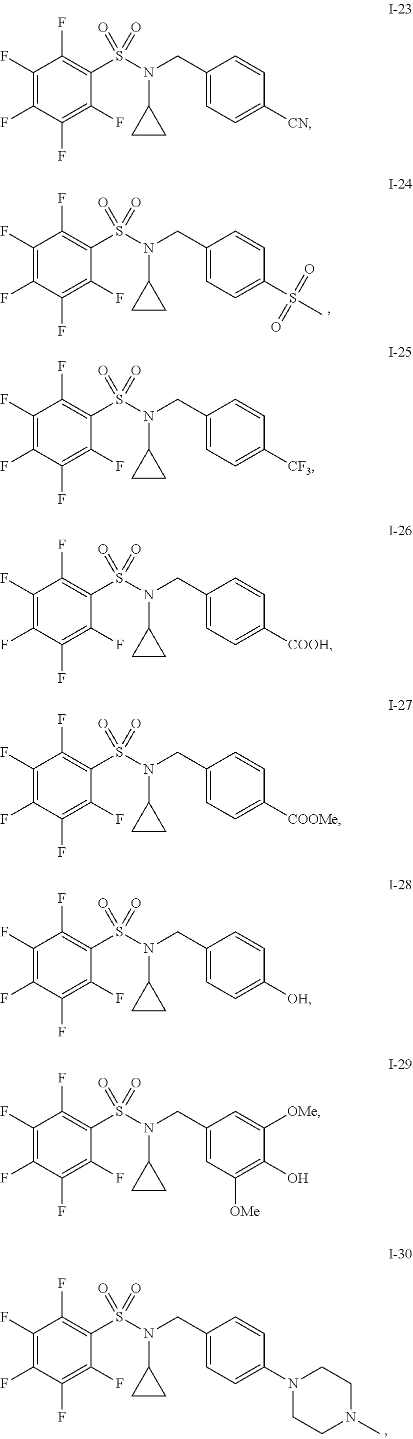

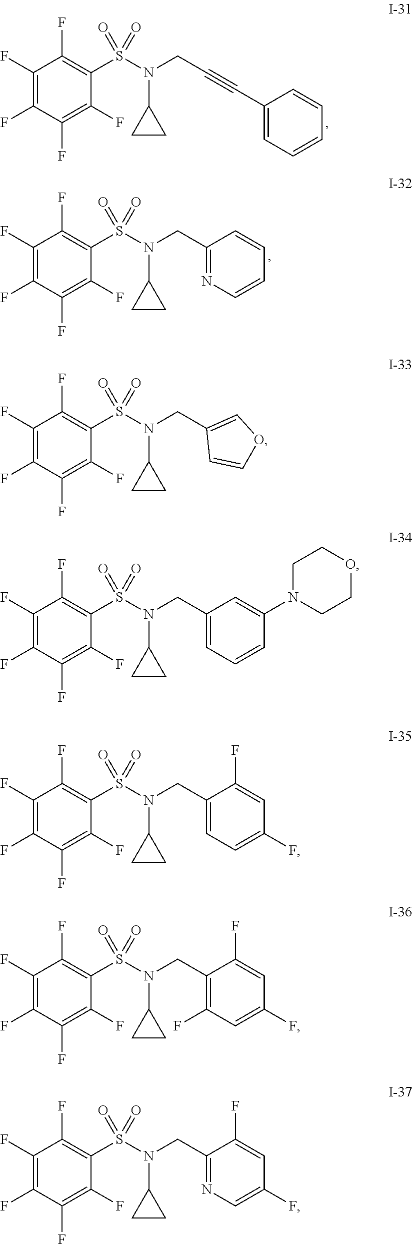

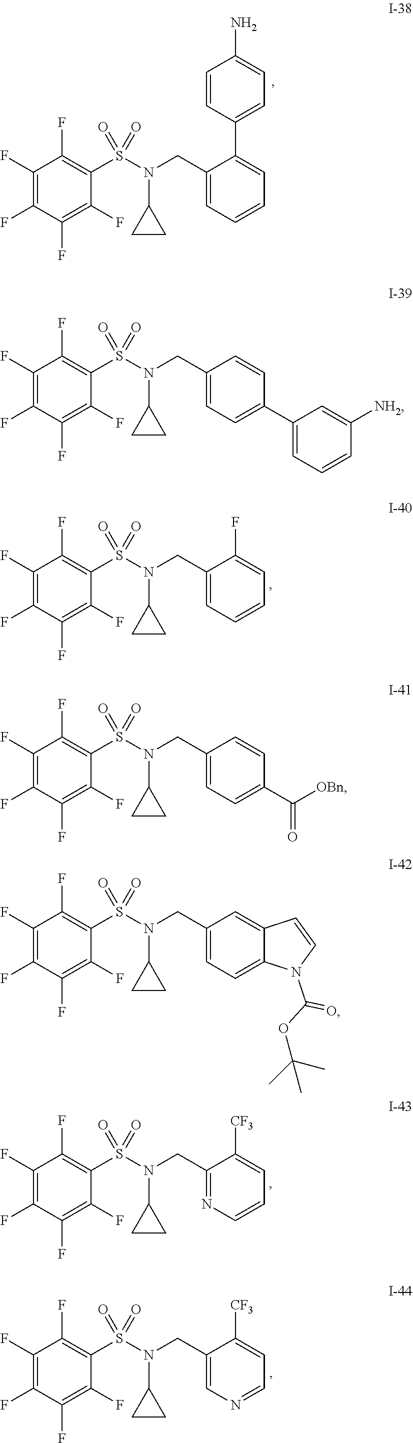

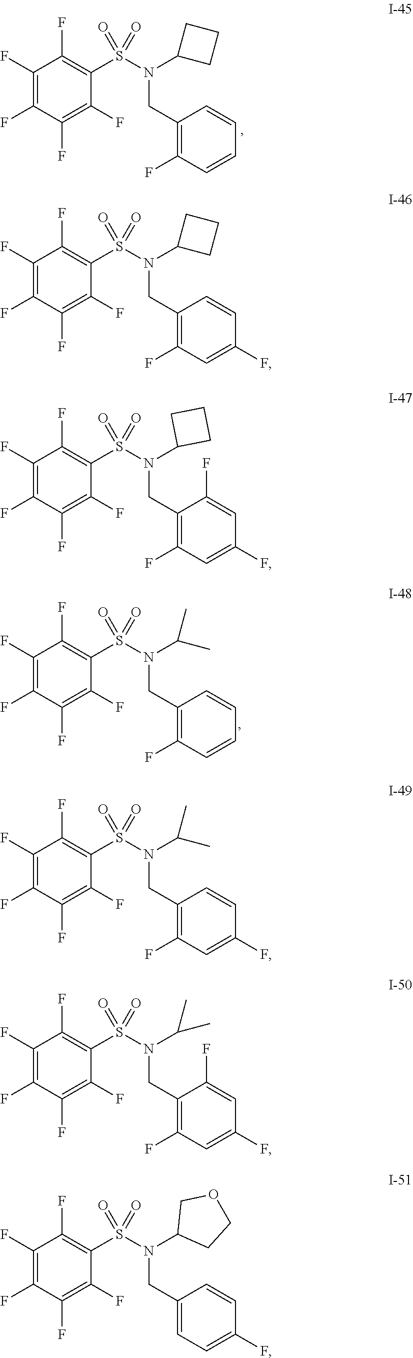

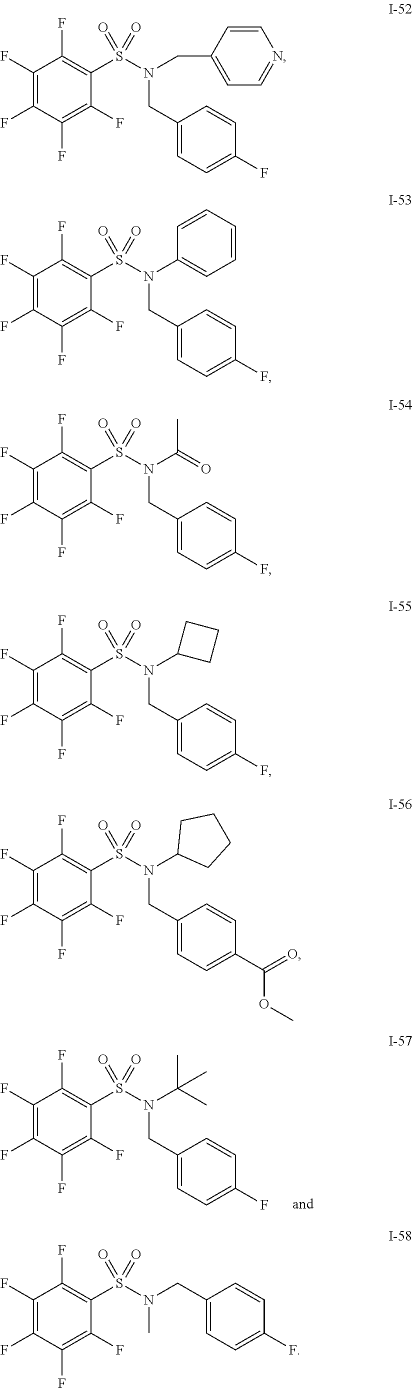

In some embodiments, the compound of Formula I has an improved metabolic stability compared to certain prior art compounds.

In another aspect, the present application includes a composition comprising one or more compounds of Formula I, and/or pharmaceutically acceptable salts and/or solvates thereof, and one or more pharmaceutically acceptable carrier or excipient.

In some embodiments, the pharmaceutical composition further comprises an additional therapeutic agent.

### III. Methods of Treatment and Medical Uses

Compounds of the present application have shown strong anticancer activity. Accordingly the present application includes the use of one or more compounds of the application or a composition of the application, as a medicament.

In some embodiments, the present application includes a method of treating a cell proliferative disorder comprising administering one or compounds of the application, or a composition of the application, to a subject in need thereof

In some embodiments, the present application includes a method of treating a disease, condition or disorder caused by uncontrolled cell proliferation comprising administering an effective amount of one or more of the compounds of this application to a subject in need thereof. In some embodiments, the disease, condition or disorder is cancer.

In some embodiments, the application includes a method of treating a subject with a cancer selected from a hematological cancer, optionally leukemia, lymphoma, or myeloma, a brain cancer, lung cancer, epidermoid cancer, ovarian cancer, or breast cancer comprising administering one or more compounds or a composition of the application. In some embodiments, the cancer is a hematological cancer, such as leukemia, lymphoma, or myeloma, or a brain cancer, such as medulloblastoma or glioblastoma.

In some embodiments, the present application includes a use of one or compounds of the application, or a composition of the application, for treating a cell proliferative disorder.

In some embodiments, the present application includes a use of one or compounds of the application, or a composition of the application, for treating a disease, condition or disorder caused by uncontrolled cell proliferation. In some embodiments, the disease, condition or disorder is cancer.

In some embodiments, the application includes a use of one or compounds of the application, or a composition of the application, for treating a subject with a cancer selected from a hematological cancer, optionally leukemia, lymphoma, or myeloma, a brain cancer, lung cancer, epidermoid cancer, ovarian cancer, or breast cancer comprising. In some embodiments, the cancer is a hematological cancer, such as leukemia, lymphoma, or myeloma, or a brain cancer, such as medulloblastoma or glioblastoma.

In some embodiments, the leukemia is acute myelomoid leukemia (AML) or acute lymphoblastic leukemia (ALL).

In some embodiments, the lymphoma is Hodgkin's or non-Hodgkin's lymphoma.

In some embodiments, the brain cancer is medulloblastoma or glioblastoma.

In some embodiments, the disease, condition, or disorder is acute myelomoid leukemia or medulloblastoma.

In some embodiments, the present application includes a method of treating a cell proliferative disorder comprising administering one or more compounds of the application, or a composition of the application, in combination with another known agent useful for treating a cell proliferative disorder to a subject in need thereof.

In some embodiments, the present application includes a method of treating a disease, condition or disorder caused by uncontrolled cell proliferation comprising administering an effective amount of one or more of the compounds of this application to a subject in combination with another known agent useful for treating a cell proliferative disorder.

In some embodiments, the present application includes a use of one or compounds of the application, or a composition of the application, for treating a cell proliferative disorder in combination with another known agent useful for treating a cell proliferative disorder.

In some embodiments, the present application includes a use of one or compounds of the application, or a composition of the application, for treating a disease, condition or disorder caused by uncontrolled cell proliferation in combination with another known agent useful for a disease, condition or disorder caused by uncontrolled cell proliferation. In some embodiments, the disease, condition or disorder is cancer.

Compounds of the present application have been shown to be capable of inhibiting UFMylation.

Accordingly, the present application includes a method for inhibiting UFMylation in a cell comprising administering an effective amount of one or more compounds of the application to the cell. The application also includes a use of one more compounds of the application for inhibiting UFMylation in a cell as well as a use of one or more compounds of the application for the preparation of a medicament for inhibiting UFMylation in a cell. The application further includes one or more compounds of the application for use in inhibiting UFMylation

As the compounds of the application have been shown to be capable of inhibiting UFMylation, the compounds of the application are useful for treating a disease, disorder or condition that benefits from inhibiting UFMylation.

Accordingly, the present application also includes a method of treating a disease, disorder or condition that benefits from inhibiting UFMylation comprising administering an effective amount of one or more compounds of the application to a subject in need thereof. The present application as includes a use of one or more compounds of the application for treatment of a disease, disorder or condition that benefits from inhibiting UFMylation as well as a use of one or more of the application for the preparation of a medicament for the treatment of a disease, disorder or condition that benefits from inhibiting UFMylation. The application further includes one or more compounds of the application for use in treating a disease, disorder or condition that benefits from inhibiting UFMylation.

In some embodiments, the disease, disorder or condition that benefits from inhibiting UFMylation is a cancer. In some embodiments, the cancer is a cancer that is caused by, or has as least as part of its etiology, upregulation of the c-Myc, pS2 and/or cyclin D1 genes.

Compounds of the present application have been shown to be capable of covalently interacting with ubiquitin-like modifier-activating enzyme 5 (UBA5).

Accordingly, the present application includes a method for covalently interacting with ubiquitin-like modifier-activating enzyme 5 (UBA5) in a cell comprising administering an effective amount of one or more compounds of the application to the cell. The application also includes a use of one more compounds of the application for covalently interacting with UBA5 in a cell as well as a use of one or more compounds of the application for the preparation of a medicament for covalently interacting with UBA5 in a cell. The application further includes one or more compounds of the application for covalently interacting with UBA5.

As the compounds of the application have been shown to be capable of covalently interacting with UBA5, the compounds of the application are useful for treating a disease, disorder or condition that benefits from covalently interacting with UBA5.

Accordingly, the present application also includes a method of treating a disease, disorder or condition that benefits from covalently interacting with UBA5 comprising administering an effective amount of one or more compounds of the application to a subject in need thereof.

The present application also includes a use of one or more compounds of the application for the treatment of a disease, disorder or condition that benefits from covalently interacting with UBA5 as well as a use of one or more of the application for the preparation of a medicament for the treatment of a disease, disorder or condition that benefits from covalently interacting with UBA5. The application further includes one or more compounds of the application for use in treating a disease, disorder or condition that benefits from covalently interacting with UBA5.

In some embodiments, the disease, disorder or condition that benefits from covalently interacting with UBA5 is a cancer dependent on UBA5 activity. In some embodiments, the cancer dependent on UBA5 activity is leukemia, bile duct, fibroblast, kidney, mesothelioma, multiple myeloma, liver, central nervous system, soft tissue, pancreas, thyroid, gastric, ovary, upper aerodigestive tract, urinary tract, lung, skin, colorectal, esophagus, breast, uterus, cervix, bone, peripheral nervous system or lymphoma.

In some embodiments, the cell is in vivo or in vitro.

In some embodiments, the subject is a mammal. In some embodiments, the subject is human.

The dosage administered will vary depending on the use and known factors such as the pharmacodynamic characteristics of the particular substance, and its mode and route of administration, age, health, and weight of the individual recipient, nature and extent of symptoms, kind of concurrent treatment, frequency of treatment, and the effect desired. Dosage regime may be adjusted to provide the optimum therapeutic response

In some embodiments, the compounds or compositions of the application are administered at least once a week. In some embodiments, the compounds or compositions are administered to the subject from about one time per two weeks, three weeks or one month. In some embodiments, the compounds or compositions are administered about one time per week to about once daily. In some embodiments, the compounds or compositions are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the one or more compounds for the uses or for the methods of the application are compound of Formula II:

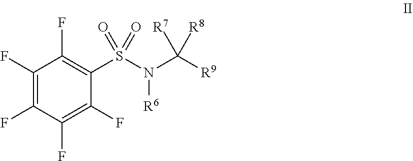

wherein:
R⁶ is selected from C₁₋₁₀alkyl, C₂₋₁₀alkenyl, C₂₋₁₀alkynyl, C(O)C₁₋₁₀alkyl, C₃₋₁₀cycloalkyl, aryl, heterocycloalkyl, heteroaryl, CH₂C₃₋₁₀cycloalkyl, CH₂aryl, CH₂heterocycloalkyl and CH₂heteroaryl, the latter 8 of which are each optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆ alkenyl, OC₂₋₆ alkynyl, C₁₋₆ alkyleneOC₁₋₆ alkyl, C₁₋₆ alkyleneOC₂₋₆ alkenyl, C₁₋₆ alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl) and NHC(O)C₁₋₆alkyl;
R⁷, and R⁸ are each independently selected from H, C₁₋₆alkyl, C₂₋₆alkenyl and C₂₋₆alkynyl; or both R⁷ and R⁸ combine to form ═O, or
R⁷ and R⁸ together with the carbon to which they are attached form C₃₋₆cycloalkyl;
R⁹ is selected from aryl, heteroaryl, heterocycloalkyl, C₃₋₁₀cycloalkyl, C≡C-aryl, C≡C-heteroaryl, and C≡C-heterocycloalkyl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆ alkyleneOC₂₋₆ alkynyl, C(O)C₁₋₆ alkyl, C(O)C₂₋₆ alkenyl, C(O)C₂₋₆ alkynyl, C(O)OC₁₋₆ alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl), NHC(O)C₁₋₆alkyl and R¹⁰;
R¹⁰ is selected from Z—C₃₋₁₀cycloalkyl, Z-heterocycloalkyl, Z-aryl and Z-heteroaryl, each of which is optionally substituted with one or more of halo, CN, OH, NH₂, ═O, CO₂H, SO₂F, C₁₋₁₀alkyl, C₂₋₁₀alkenyl, C₂₋₁₀ alkynyl, NH(C₁₋₆ alkyl), N(C₁₋₆ alkyl)(C₁₋₆ alkyl), OC₁₋₆ alkyl, OC₂₋₆ alkenyl, OC₂₋₆ alkynyl, C₁₋₆ alkyleneOC₁₋₆ alkyl, C₁₋₆ alkyleneOC₂₋₆ alkenyl, C₁₋₆ alkyleneOC₂₋₆ alkynyl, C(O)C₁₋₆ alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl), NHC(O)C₁₋₆ alkyl, C₃₋₁₀ cycloalkyl, aryl, heteroaryl and heterocycloalkyl, the latter four groups being further optionally substituted by C₁₋₆alkyl, C(O)C₁₋₆alkyl and benzyl;
x is 0, 1 or 2;
Z is selected from a direct bond, C₁₋₄ alkylene, O, NH, S, SO and SO₂ and
all alkyl, alkenyl, alkynyl, aryl, heteroaryl, heterocycloalkyl and alkylene groups are optionally halosubstituted.

In some embodiments, the one or more compounds for the uses or for the methods of the application are compound of Formula II:

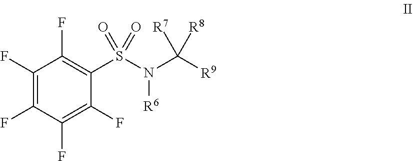

wherein:
R⁶ is selected from C₃₋₁₀alkyl, C₃₋₁₀alkenyl, C₃₋₁₀alkynyl, C(O)C₁₋₁₀alkyl, C₃₋₁₀cycloalkyl, aryl, heterocycloalkyl, heteroaryl, CH₂C₃₋₁₀cycloalkyl, CH₂aryl and CH₂heterocycloalkyl, the latter 7 of which are each optionally substituted with one or more of halo, CN, OH, NH₂, ═O, CO₂H, SO₂F C₁₋₁₀alkyl, C₂₋₁₀alkenyl, C₂₋₁₀alkenyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆ alkyl, S(O)ₓC₂₋₆ alkenyl, S(O)ₓC₂₋₆ alkynyl C(O)NHC₁₋₆ alkyl, C(O)N(C₁₋₆ alkyl(C₁₋₆ alkyl) and NHC(O)C₁₋₆alkyl;
R⁷, and R⁸ are each independently selected from H, C₁₋₆alkyl, C₂₋₆alkenyl and C₂₋₆alkynyl; or both R⁷ and R⁸ combine to form ═O, or
R⁷ and R⁸ together with the carbon to which they are attached form C₃₋₆cycloalkyl;
R⁹ is selected from aryl, heteroaryl, heterocycloalkyl and C₃₋₁₀cycloalkyl, each of which is optionally substituted with one or more of halo, CN, OH, NH₂, CO₂H, SO₂F, C₁₋₁₀alkyl, C₂₋₁₀alkenyl, C₂₋₁₀alkenyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)_{y}C₁₋₆alkyl, S(O)_{y}C₂₋₆alkenyl, S(O)_{y}C₂₋₆alkynyl C(O)NHC₁₋₆alkyl, C(O)N(C₁₋₆alkyl(C₁₋₆alkyl), NHC(O)C₁₋₆alkyl and R¹⁰;
R¹⁰ is selected from Z′—C₃₋₁₀cycloalkyl, Z′-heterocycloalkyl, Z′-aryl and Z′-heteroaryl, each of which is optionally substituted with one or more of halo, CN, OH, NH₂, ═O, CO₂H, SO₂F, C₁₋₁₀alkyl, C₂₋₁₀alkenyl, C₂₋₁₀alkenyl, NH(C₁₋₆ alkyl), N(C₁₋₆ alkyl)(C₁₋₆ alkyl), OC₁₋₆ alkyl, OC₂₋₆ alkenyl, OC₂₋₆alkynyl, C₁₋₆ alkyleneOC₁₋₆ alkyl, C₁₋₆ alkyleneOC₂₋₆ alkenyl, C₁₋₆ alkyleneOC₂₋₆ alkynyl, C(O)C₁₋₆alkyl, C(O)C₂₋₆alkenyl, C(O)C₂₋₆alkynyl, C(O)OC₁₋₆alkyl, C(O)OC₂₋₆alkenyl, C(O)OC₂₋₆alkynyl, S(O)ₓC₁₋₆ alkyl, S(O)ₓC₂₋₆ alkenyl, S(O)ₓC₂₋₆ alkynyl C(O)NHC₁₋₆ alkyl, C(O)N(C₁₋₆ alkyl(C₁₋₆ alkyl), NHC(O)C₁₋₆alkyl, C₃₋₁₀cycloalkyl, aryl, heteroaryl and heterocycloalkyl, the latter four groups being further optionally substituted by C₁₋₆alkyl, C(O)C₁₋₆alkyl and benzyl;
y is 0, 1 or 2;
Z′ is selected from a direct bond, C₁₋₄alkylene, O, NH, S, SO and SO₂ and all alkyl, alkenyl, alkynyl, aryl, heteroaryl, heterocycloalkyl and alkylene groups are optionally halosubstituted.

### IV. Methods of Making Compounds of the Application

In some embodiments, compounds with the generic structure I and II are prepared as shown in Scheme 1 by reacting an appropriate starting amine of Formula A in a solvent such as dichloromethane (DCM) or chloroform in the presence of a base such as N,N-diisopropylethylamine (DIPEA) or triethylamine (TEA) and pentafluorophenylsulfonyl chloride (B). In some embodiments, this reaction is carried out at 0° C., and is slowly warmed to an ambient temperature. The desired product of this reaction (compounds of Formula C) is then reacted with an appropriate reagent of Formula D, in which X is a suitable leaving group such as bromide, in a solvent such as dimethylforamide (DMF) and in the presence of a base such as DIPEA or TEA to yield the compounds with the generic structure I and II.

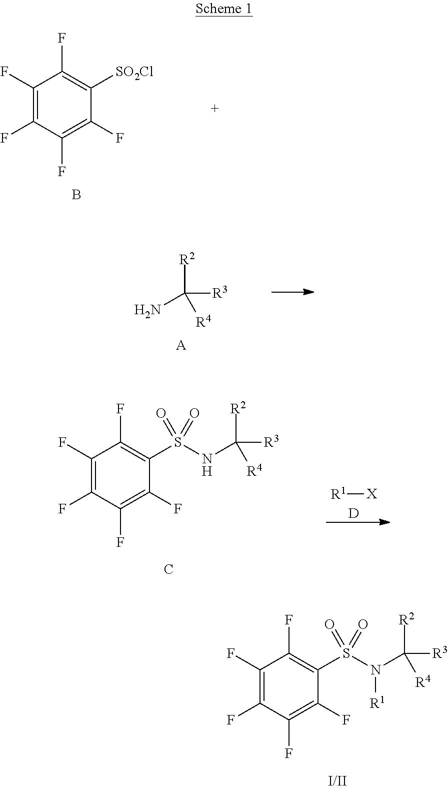

Alternatively, as shown in Scheme 2, compounds with the generic structure I and II, wherein R² and R³ are H, may also be prepared by reacting appropriate starting amine (E) in a solvent such as 1,2-dichloroethane (DCE) with an appropriate aldehyde (F) and sodium triacetoxyborohydride. In some embodiments, this reaction is carried out at ambient temperature. The desired secondary amine product (G) is then reacted in a solvent such as dichloromethane (DCM) or chloroform in the presence of a base such as N,N-diisopropylethylamine or triethylamine and pentafluorophenylsulfonyl chloride (B). In some embodiments, this reaction is carried out at 0° C., and slowly warmed to an ambient temperature.

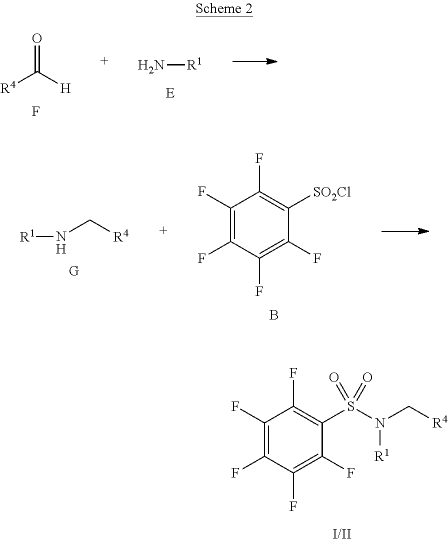

In another alternative, as shown in Scheme 3, compounds with the generic structure I and II wherein R² and R³ are H are prepared as shown in Scheme 3 by reacting pentafluorophenylsulfonyl chloride (B) in a solvent such as dichloromethane (DCM) or chloroform in the presence of a base such as N,N-diisopropylethylamine (DIPEA) or triethylamine (TEA) and appropriate starting amine of Formula E. In some embodiments, this reaction is carried out at 0° C., and is slowly warmed to an ambient temperature. The desired product of this reaction (compounds of Formula H) is then reacted with an appropriate reagent of Formula J, in which X is a suitable leaving group such as bromide, in a solvent such as dimethylforamide (DMF) and in the presence of a base such as cesium carbonate to yield the compounds with the generic structure I and II, wherein R² and R³ are H.

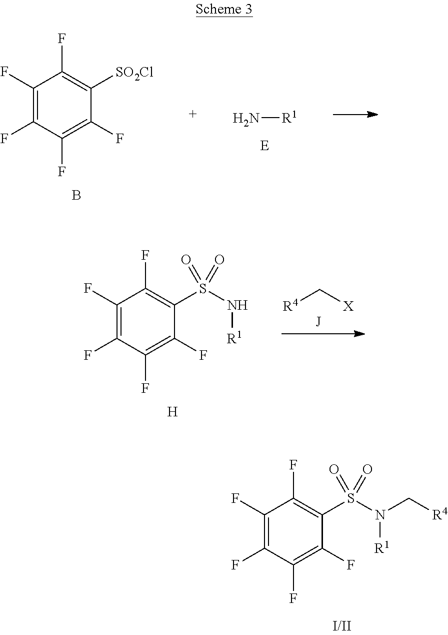

### V. Examples

### A. General Methods

Exemplary compounds of the application were synthesized using the methods described herein, or other methods, which are known in the art. Unless otherwise noted, reagents and solvents were obtained from commercial suppliers.

Anhydrous solvents methanol, dichloromethane (CH₂Cl₂, DCM), tetrahydrofuran (THF) and dimethylformamide (DMF) were purchased from Sigma Aldrich and used directly from Sure-Seal bottles. All reactions were performed under an atmosphere of dry nitrogen in oven-dried glassware and were monitored for completeness by thin-layer chromatography (TLC) using silica gel (visualized by UV light, or developed by treatment with KMnO₄ stain). NMR spectra were recorded in Bruker Avance III spectrometer at 23° C., operating at 400 MHz for ¹H NMR and 100 MHz ¹³C NMR spectroscopy either in CDCl₃, CD₃OD or d₆-DMSO. Chemical shifts (d) are reported in parts per million (ppm) after calibration to residual isotopic solvent. Coupling constants (J) are reported in Hz. Mass spectrometry was performed with an AB/Sciex QStar mass spectrometer with an ESI source, MS/MS and accurate mass capabilities, associated with an Agilent 1100 capillary LC system. Before biological testing, inhibitor purity was evaluated by reversed-phase HPLC (rpHPLC). Analysis by rpHPLC was performed using a Phenomenex Luna 5u C18 150 mm×4.6 mm column run at 1.2 mL/min, and using gradient mixtures. The linear gradient consisted of a changing solvent composition of either (1) 15% MeCN and 85% HO with 0.1% TFA (v/v) to 100% MeCN over 30 minutes and (II) 15% MeCN and 85% H₂O with 0.1% TFA (v/v) to 100 MeCN over 60 minutes, UV detection at 250 nm. For reporting HPLC data, percentage purity is given in parentheses after the retention time for each condition. All biologically evaluated compounds are >95% chemical purity as measured by HPLC. The HPLC traces for all tested compounds are provided in supporting information.

### B. Synthesis of Compounds

### Example 1 Synthesis of N-cyclopropyl-2,3,4,5,6-pentafluoro-N-(4-fluorobenzyl)benzenesulfonamide (I-1)

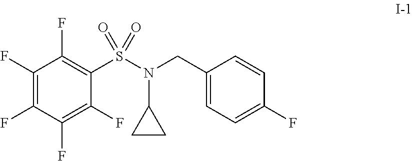 [SMILES: O=S(=O)(c1c(F)c(F)c(F)c(F)c1F)N(Cc1ccc(F)cc1)C1CC1]

To a solution of 2,3,4,5,6-pentafluorobenzenesulfonyl chloride (1.0 equiv) in DCM (0.1 M) stirring at 0° C. were added cyclopropanamine (1.0 equiv) and DIPEA (2.2 equiv) in a dropwise matter. The reaction mixture was allowed to gradually warm to room temperature and the progress of the reaction was monitored by TLC. Upon completion, the reaction was quenched by the addition of DCM and 0.1 M HCl, and transferred to a separatory funnel. The two layers were partitioned and the aqueous layer was extracted with DCM (3×). Combined organic fractions were dried over MgSO₄ and concentrated in vacuo. The crude sample was absorbed onto a small amount of silica and purified using a gradient of Hexanes:Ethyl Acetate. N-cyclopropyl-2,3,4,5,6-pentafluoro-benzenesulfonamide was isolated as a white solid (75%). ¹H NMR (400 MHz, CDCl₃) δ 2.45 (m, 1H), 0.78 (m, 4H).

To a solution of N-cyclopropyl-2,3,4,5,6-pentafluoro-N-(4-fluorobenzyl)benzenesulfonamide (1.0 equiv.) and cesium carbonate (2.1 equiv.) in DMF (0.1 M) was added 1-(bromomethyl)-4-fluorobenzene (1.2 equiv.). Upon completion as indicated by TLC, the reaction was quenched by the addition of DCM and 0.1 M HCl, and transferred to a separatory funnel. The two layers were partitioned and the aqueous layer was extracted with DCM (3×). Combined organic fractions were washed with a satured solution of brine (3×), dried over MgSO₄ and concentrated in vacuo. The crude sample was purified using prep-HPLC and was lyophilized from water/acetonitrile to afford a white powder (70%). ¹H NMR (400 MHz, CDCl₃) δ 7.35 (dd, J=8.3, 5.4 Hz, 2H), 7.03 (t, J=8.4 Hz, 2H), 4.51 (s, 2H), 2.34 (m, 1H), 0.69 (s, J=7.5 Hz, 4H).

### Example 2 N-(cyclopropylmethyl)-2,3,4,5,6-pentafluoro-N-(4-fluorobenzypenzenesulfonamide

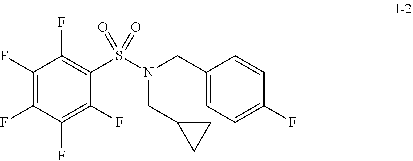 [SMILES: O=S(=O)(c1c(F)c(F)c(F)c(F)c1F)N(Cc1ccc(F)cc1)CC1CC1]

2,3,4,5,6-pentafluoro-N-(cyclopropylmethyl)benzenesulfonamide was prepared in an analogous manner described in Example 1, and was isolated as a white solid (65%). ¹H NMR (400 MHz, CDCl₃) δ 0.20 (dt, J=6.1, 4.8 Hz, 2H), 0.43-0.56 (m, 2H), 0.86-1.05 (m, 1H), 3.06 (dd, J=7.2, 5.8 Hz, 2H), 5.58 (t, J=5.8 Hz, 1H).

(N-(cyclopropylmethyl)-2,3,4,5,6-pentafluoro-N-(4-fluorobenzyl)benzenesulfonamide was prepared in an analogous manner described in Example 1, and was isolated as a free-flowing white powder (70%). ¹H NMR (400 MHz, CDCl₃) δ 7.35 (dd, J=8.5, 5.3 Hz, 2H), 7.06 (t, J=8.6 Hz, 2H), 4.64 (s, 2H), 3.21 (d, J=7.0 Hz, 2H), 0.83-0.70 (m, 1H), 0.43 (dt, J=7.9, 5.4 Hz, 2H), 0.08 (q, J=4.9 Hz, 2H).

### Example 3 Synthesis of N-benzyl-2,3,4,5,6-pentafluoro-N-(4-fluorobenzypenzenesulfonamide

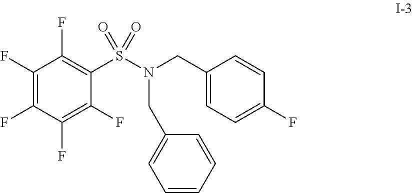 [SMILES: O=S(=O)(c1c(F)c(F)c(F)c(F)c1F)N(Cc1ccccc1)Cc1ccc(F)cc1]

A solution of 4-fluorobenzaldehyde (1.0 equiv.), phenylmethanamine (1.1 equiv.), acetic acid (1.5 equiv.) were dissolved in anhydrous DCE (0.1 M). The mixture was stirred for 1 h at room temperature, followed by the portion-wise addition of sodium triacetoxyborohydride (1.5 equiv). Upon complete consumption of the aldehyde as indicated by TLC, the reaction was diluted with DCM and transferred to a separatory funnel with a saturated solution of NaHCO₃. The two layers were partitioned and the aqueous layer was extracted with DCM (3×). Combined organic fractions were washed with a saturated solution of sodium chloride, dried over MgSO₄ and concentrated. The crude sample was adsorbed onto a small amount of silica and purified by column chromatography eluting with a gradient of Hexanes:Ethyl acete. N-benzyl-1-(4-fluorophenyl)methanamine was isolated as a beige oil (68%). ¹H NMR (400 MHz, CDCl3) δ 7.37-7.28 (m, 7H), 7.07-7.01 (m, 2H), 3.83 (s, 2H), 3.80 (s, 2H).

N-(cyclopropylmethyl)-2,3,4,5,6-pentafluoro-N-(4-fluorobenzyl)benzenesulfonamide was prepared in an analogous manner described in Example 1, and was isolated as a white powder (77%). ¹H NMR (400 MHz, CDCl₃) δ 7.32-7.25 (m, 3H), 7.22-7.12 (m, 4H), 6.99 (t, J=8.6 Hz, 2H), 4.54 (s, 2H), 4.51 (s, 2H).

### Example 4 Synthesis of N-(4-fluorobenzyl)-N-((perfluorophenyl)sulfonyl)acetamide

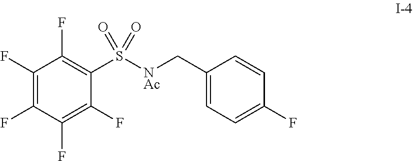

A solution of 2,3,4,5,6-pentafluoro-N-(4-fluorobenzyl)benzenesulfonamide (1.0 equiv.) in DMF (0.1 M) was cooled to 0° C. followed by the addition of DIPEA (1.5 equiv.) and acetyl chloride (1.2 equiv.). The reaction mixture was gradually warmed to room temperature and allowed to stir for 6 hours. The reaction mixture was quenched by the addition of water and diluted further with DCM. The two layers were partitioned and the aqueous layer was extracted with DCM (3×). Combined organic fractions were washed with a saturated solution of sodium chloride, dried over MgSO₄ and concentrated down. The crude sample was purified using prep-HPLC and was lyophilized from water/acetonitrile affording a white powder (68%). ¹H NMR (400 MHz, CDCl₃) δ 7.39 (dd, J=8.6, 5.3 Hz, 2H), 7.07 (t, J=8.6 Hz, 2H), 5.06 (s, 2H), 2.35 (s, 3H).

### Example 5 Synthesis of N-cyclopentyl-2,3,4,5,6-pentafluoro-N-(4-fluorobenzyl)benzenesulfonamide

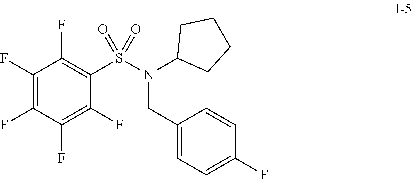 [SMILES: O=S(=O)(c1c(F)c(F)c(F)c(F)c1F)N(Cc1ccc(F)cc1)C1CCCC1]

N-cyclopentyl-2,3,4,5,6-pentafluoro-N-(4-fluorobenzyl)benzenesulfonamide was prepared in an analogous manner described in Example 1, and was isolated as a white powder. ¹H NMR (400 MHz, CDCl₃) δ 7.35 (dd, J=8.6, 5.3 Hz, 2H), 7.05-6.99 (m, 2H), 4.52 (s, 2H), 4.41-4.31 (m, 1H), 1.83-1.72 (m, 2H), 1.68-1.57 (m, 2H), 1.57-1.40 (m, 4H).

### Example 6 Synthesis of N-benzyl-N-cyclopropyl-2,3,4,5,6-pentafluorobenzenesulfonamide

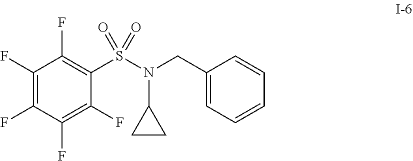 [SMILES: O=S(=O)(c1c(F)c(F)c(F)c(F)c1F)N(Cc1ccccc1)C1CC1]

N-benzyl-N-cyclopropyl-2,3,4,5,6-pentafluorobenzenesulfonamide was prepared in an analogous manner described in Example 1, and was isolated as a white powder (80%). ¹H NMR (400 MHz, CDCl₃) δ 7.41-7.29 (m, 5H), 4.58 (s, 2H), 2.49-2.37 (m, 1H), 0.78-0.68 (m, 4H).

### Example 7 Synthesis of N-(4-chlorobenzyl)-N-cyclopropyl-2,3,4,5,6-pentafluorobenzenesulfonamide

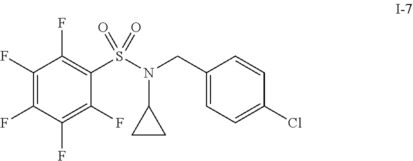 [SMILES: O=S(=O)(c1c(F)c(F)c(F)c(F)c1F)N(Cc1ccc(Cl)cc1)C1CC1]

N-(4-chlorobenzyl)-N-cyclopropyl-2,3,4,5,6-pentafluorobenzenesulfonamide was prepared in an analogous manner described in Example 1, and was isolated as a white powder (88%). ¹H NMR (400 MHz, CDCl₃) δ 7.31 (s, 4H), 4.51 (s, 2H), 2.40-2.28 (m, 1H), 0.69 (s, 4H).

### Example 8 Synthesis of N-cyclopropyl-2,3,4,5,6-pentafluoro-N-(4-methoxybenzyl)benzenesulfonamide

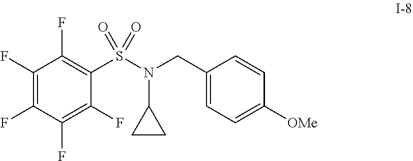 [SMILES: COc1ccc(CN(C2CC2)S(=O)(=O)c2c(F)c(F)c(F)c(F)c2F)cc1]

N-(4-methoxybenzyl)cyclopropanamine was prepared in an analogous manner described in Example 1, and was isolated as beige oil (90%): ¹H NMR (400 MHz, CDCl₃) δ 7.26 (d, J=8.6 Hz, 2H), 6.88 (d, J=8.6 Hz, 2H), 3.82 (s, 3H), 2.21-2.12 (m, 1H), 0.52-0.36 (m, 4H).

N-cyclopropyl-2,3,4,5,6-pentafluoro-N-(4-methoxybenzyl)benzenesulfonamide was prepared in an analogous manner described in Example 1, and was isolated as a white solid (75%). ¹H NMR (400 MHz, CDCl₃) δ 7.31 (d, J=8.6 Hz, 2H), 6.88 (d, J=8.6 Hz, 2H), 4.51 (s, 2H), 3.83 (s, 3H), 2.41-2.33 (m, 1H), 0.74-0.70 (m, 4H).

### Example 9 Synthesis of N-cyclopropyl-2,3,4,5,6-pentafluoro-N-(pyridin-4-ylmethyl)benzenesulfonamide

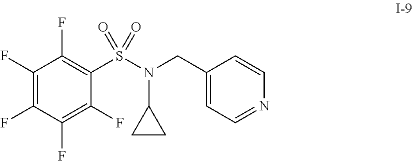 [SMILES: O=S(=O)(c1c(F)c(F)c(F)c(F)c1F)N(Cc1ccncc1)C1CC1]

N-cyclopropyl-2,3,4,5,6-pentafluoro-N-(pyridin-4-ylmethyl)benzenesulfonamide was prepared in an analogous manner described in Example 1, and was isolated as a beige powder (55%). ¹H NMR (400 MHz, CDCl₃) δ 8.63 (d, J=4.8 Hz, 2H), 7.33 (d, J=5.2 Hz, 2H), 4.58 (s, 2H), 2.44 (p, J=5.3 Hz, 1H), 0.75-0.70 (m, 4H).

### Example 10 Synthesis of N-cyclopropyl-2,3,4,5,6-pentafluoro-N-(2,2,2-trifluoro-1-(4 fluorophenyl)ethyl)benzenesulfonamide

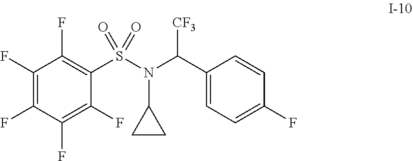 [SMILES: O=S(=O)(c1c(F)c(F)c(F)c(F)c1F)N(C1CC1)C(c1ccc(F)cc1)C(F)(F)F]

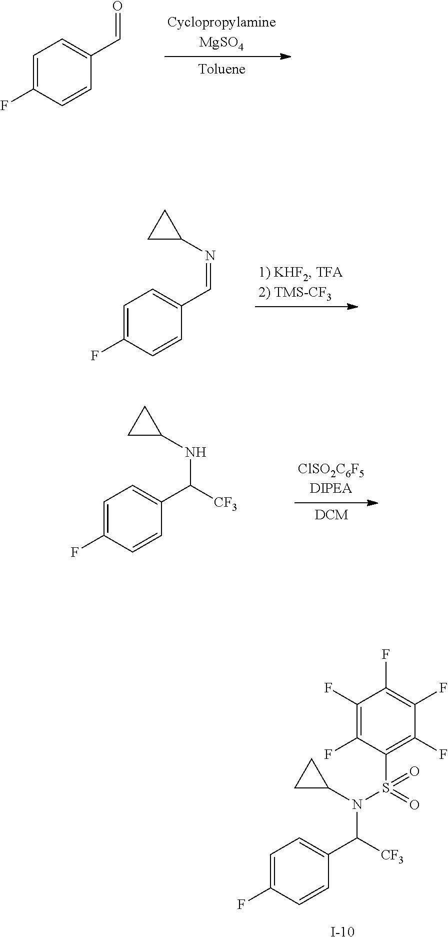

An oven dried round bottom flask flushed with N₂ was charged with 4-fluorobenzaldehyde (2.0 mmol), cyclopropylamine (2.4 mmol), MgSO₄ (5.0 mmol) and toluene (0.5 M). The reaction mixture was stirred at room temperature for 18 hours. The reaction was filtered and concentrated. Crude ¹H NMR analysis showed complete conversion to imine and was used without further purification (298 mg, 91%). (E)-N-cyclopropyl-1-(4-fluorophenyhmethanimine ¹H NMR (400 MHz, CDCl₃) δ 8.41 (s, 1H), 7.70-7.63 (m, 2H), 7.07 (td, J=8.5, 1.4 Hz, 2H), 3.07-2.96 (m, 1H), 1.01-0.90 (m, 4H).

An oven dried round bottom flask equipped with a stir bar and flushed with N₂ was charged with (E)-N-cyclopropyl-1-(4-fluorophenyl)methanimine (1.12 mmol), KHF₂ (0.84 mmol), DMF (3.35 mmol) and MeCN (0.5 M). The reaction mixture was cooled to 0° C. followed by the dropwise addition of trifluoroacetic acid (TFA) (1.39 mmol). The resulting mixture was stirred for 5 minutes, then CF₃SiMe₃ was added, the cooling bath was removed and the reaction mixture was stirred at room temperature for 3 hours. The reaction was quenched by the dropwise addition of saturated aqueous Na₂CO₃ (0.5 mL), which was allowed to stir for an additional 2 minutes. The mixture was further diluted with water and extracted diethyl ether (Et₂O) (3×). Combined organic fractions were dried over MgSO₄ and concentrated in vacuo. N-(2,2,2-trifluoro-1-(4-fluorophenyl)ethyl)cyclopropanamine was isolated as colourless oil (44%): ¹H NMR (400 MHz, Chloroform-d) δ 7.39 (dd, J=8.5, 5.3 Hz, 2H), 7.13-7.08 (m, 2H), 4.21 (q, J=7.7 Hz, 1H), 2.16-2.11 (m, 1H), 0.45 (m, 4H).

A solution of pentafluorobenzenesulfonyl chloride (1.91 mmol) in DCM (0.2 M) was cooled to 0° C. N-(2,2,2-trifluoro-1-(4-fluorophenyl)ethyl)cyclopropanamine (0.71 mmol) and DIPEA (4.79 mmol) were subsequently added dropwise and the reaction mixture was gradually warmed to rt. The reaction was allowed to stir at room temperature until completion, as indicated by TLC. The reaction was quenched by the addition of 0.1 M HCl, and the two layers were partitioned. The aqueous layer was extracted with DCM (3×) and combined organic fractions were washed with brine and dried over MgSO₄ and concentrated in vacuo. The crude sample was absorbed onto a small amount of silica and purified using flash chromatography using an EtOAc:Hex gradient. N-cyclopropyl-2,3,4,5,6-pentafluoro-N-(2,2,2-trifluoro-1-(4-fluorophenyl)ethyl)benzenesulfonamide was isolated as white solid (30%). ¹H NMR (400 MHz, CDCl₃) δ 7.67 (dd, J=8.6, 5.2 Hz, 2H), 7.22-7.08 (m, 2H), 5.52 (q, J=8.9 Hz, 1H), 2.28 (tt, J=7.0, 3.8 Hz, 1H), 1.07 (dq, J=13.3, 4.4, 3.6 Hz, 1H), 0.93-0.82 (m, 2H), 0.80-0.67 (m, 1H).

### Example 11 Synthesis of N-cyclopropyl-2,3,4,5,6-pentafluoro-N-(3-fluorobenzyl)benzenesulfonamide

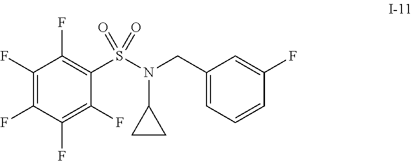 [SMILES: O=S(=O)(c1c(F)c(F)c(F)c(F)c1F)N(Cc1cccc(F)c1)C1CC1]

N-cyclopropyl-2,3,4,5,6-pentafluoro-N-(3-fluorobenzyl)benzenesulfonamide was prepared in an analogous manner described in Example 1, and was isolated as a white powder (78%). ¹H NMR (400 MHz, CDCl₃) δ 7.34 (td, J=7.9, 5.9 Hz, 1H), 7.18 (d, J=7.7 Hz, 1H), 7.11 (d, J=9.5 Hz, 1H), 7.03 (td, J=8.4, 2.4 Hz, 1H), 4.55 (s, 2H), 2.45-2.36 (m, 1H), 0.73 (d, J=5.3 Hz, 4H).

### Example 12 Synthesis of N-cyclopropyl-2,3,4,5,6-pentafluoro-N-((6-fluoropyridin-3-yl)methyl)benzenesulfonamide

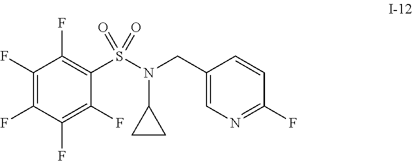 [SMILES: O=S(=O)(c1c(F)c(F)c(F)c(F)c1F)N(Cc1ccc(F)nc1)C1CC1]

N-((6-fluoropyridin-3-yl)methyl)cyclopropanamine was prepared in an analogous manner described in Example 1, and was isolated as an oil (60%): ¹H NMR (400 MHz, MeOD) δ 8.15 (d, J=2.5 Hz, 1H), 7.93 (td, J=8.1, 2.5 Hz, 1H), 7.03 (dd, J=8.5, 2.5 Hz, 1H), 2.10 (tt, J=7.0, 3.7 Hz, 1H), 0.53-0.24 (m, 5H).

N-cyclopropyl-2,3,4,5,6-pentafluoro-N-((6-fluoropyridin-3-yl)methyl)benzenesulfonamide was prepared in an analogous manner described in Example 1, and was isolated as a beige powder (66%). ¹H NMR (400 MHz, CDCl₃) δ 8.21 (d, J=2.5 Hz, 1H), 7.95 (td, J=8.0, 2.6 Hz, 1H), 6.99 (dd, J=8.5, 3.0 Hz, 1H), 4.57 (s, 2H), 2.38-2.30 (m, 1H), 0.80-0.64 (m, 5H).

### Example 13 Synthesis of N-cyclopropyl-2,3,4,5,6-pentafluoro-N-((5-fluoropyridin-2-yl)methyl)benzenesulfonamide

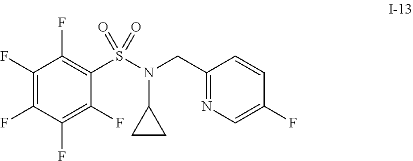 [SMILES: O=S(=O)(c1c(F)c(F)c(F)c(F)c1F)N(Cc1ccc(F)cn1)C1CC1]

N-((5-fluoropyridin-2-yl)methyl)cyclopropanamine was prepared in an analogous manner described in Example 1, and was isolated as an oil (66%).

N-cyclopropyl-2,3,4,5,6-pentafluoro-N-((5-fluoropyridin-2-yl)methyl)benzenesulfonamide was prepared in an analogous manner described in Example 1, and was isolated as a beige powder (66%). ¹H NMR (400 MHz, CDCl₃) δ 8.30 (d, J=2.5 Hz, 1H), 7.49-7.40 (m, 2H), 4.70 (s, 2H), 2.54 (tt, J=7.0, 3.7 Hz, 1H), 0.87-0.70 (m, 5H).

### Example 14 Synthesis of N-(4-bromobenzyl)-N-cyclopropyl-2,3,4,5,6-pentafluorobenzenesulfonamide

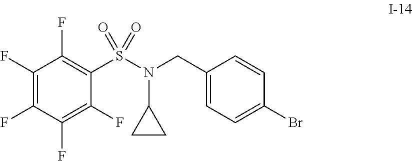 [SMILES: O=S(=O)(c1c(F)c(F)c(F)c(F)c1F)N(Cc1ccc(Br)cc1)C1CC1]

N-(4-bromobenzyl)-N-cyclopropyl-2,3,4,5,6-pentafluorobenzenesulfonamide was prepared in an analogous manner described in Example 1, and was isolated as a beige powder (81%). ¹H NMR (400 MHz, CDCl3) δ 7.49 (d, J=8.3 Hz, 1H), 7.28 (d, J=8.3 Hz, 1H), 4.52 (s, 1H), 2.38 (p, J=5.4 Hz, 1H), 0.82-0.63 (m, 2H).

### Example 15 Synthesis of N-cyclopropyl-2,3,4,5,6-pentafluoro-N-((4′-(methylsulfonyl)-[1,1′-biphenyl]-4-yl)methyl)benzenesulfonamide

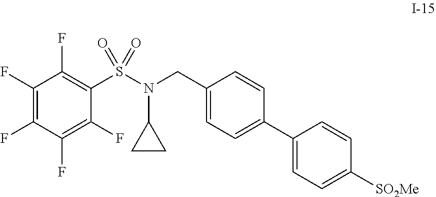 [SMILES: CS(=O)(=O)c1ccc(-c2ccc(CN(C3CC3)S(=O)(=O)c3c(F)c(F)c(F)c(F)c3F)cc2)cc1]

N-(4-bromobenzyl)-N-cyclopropyl-2,3,4,5,6-pentafluorobenzenesulfonamide (0.0873 mmol), 4-(methylsulfonyl)phenylboronic acid (0.096 mmol), tricyclohexylphosphine (0.00873 mmol) and potassium phosphate (0.306 mmol) were added to a round bottom flask equipped with a stir bar. The mixture dissolved in toluene and purged with argon for 10 minutes. To this reaction mixture was added 0.05 mL of water, and the resulting solution was allowed to stir for 5 minutes. Pd(OAc)₂ (1 mg, 0.00436 mmol) was then added and the resulting reaction mixture was allowed to stir at 100° C. for 12 hours. The reaction was quenched by the addition of water and the aqueous phase was extracted with EtOAc three times. The collected organic phase was washed with a saturated solution of sodium chloride, dried with MgSO₄, and concentrated in vacuo. The title compound was isolated using prep-HPLC and was lyophilized from water/acetonitrile to afford a white powder (22%). ¹H NMR (400 MHz, CDCl3) δ 8.16-7.98 (m, 2H), 7.91-7.74 (m, 2H), 7.65-7.59 (m, 2H), 7.56-7.49 (m, 2H), 4.64 (s, 2H), 3.13 (s, 3H), 2.46 (p, J=5.4 Hz, 1H), 0.76 (m, 4H).

### Example 16 Synthesis of 2,3,4,5,6-pentafluoro-N-(4-fluorobenzyl)-N-isopropylbenzenesulfonamide

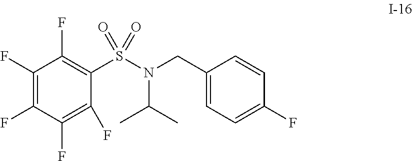 [SMILES: CC(C)N(Cc1ccc(F)cc1)S(=O)(=O)c1c(F)c(F)c(F)c(F)c1F]

2,3,4,5,6-pentafluoro-N-(4-fluorobenzyl)-N-isopropylbenzenesulfonamide was prepared in an analogous manner described in Example 1, and was isolated as a white powder (44%). ¹H NMR (400 MHz, CDCl₃) δ 7.39-7.31 (m, 2H), 7.06-6.98 (m, 2H), 4.52 (s, 2H), 4.41-4.29 (m, 1H), 1.14 (d, J=6.8 Hz, 6H).

### Example 17 Synthesis of N-cyclopropyl-N-(4-fluorobenzyl)benzenesulfonamide

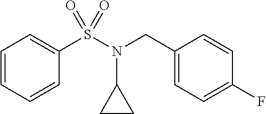 [SMILES: O=S(=O)(c1ccccc1)N(Cc1ccc(F)cc1)C1CC1]

### Comparative Example 2

N-cyclopropylbenzenesulfonamide was prepared in an analogous manner described in Example 1, and was isolated as a colourless oil (95%). ¹H NMR (400 MHz, CDCl₃) δ 7.94-7.92 (m, 2H), 7.62-7.52 (m, 3H), 5.14 (s, 1H), 2.26 (td, J=3.8, 3.2, 1.6 Hz, 1H), 0.64-0.59 (m, 4H).

N-cyclopropyl-N-(4-fluorobenzyl)benzenesulfonamide was prepared in an analogous manner described in Example 1, and was isolated as a white solid (59%). ¹H NMR (400 MHz, CDCl₃) δ 7.88-7.80 (m, 2H), 7.61-7.55 (m, 1H), 7.54-7.47 (m, 2H), 7.35-7.25 (m, 2H), 7.01-6.92 (m, 2H), 4.32 (s, 2H), 2.03-1.95 (m, 1H), 0.68-0.50 (m, 4H).

### Example 18 Synthesis of N-cyclopropyl-4-fluoro-N-(4-fluorobenzyl)benzenesulfonamide

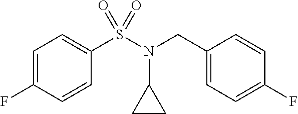 [SMILES: O=S(=O)(c1ccc(F)cc1)N(Cc1ccc(F)cc1)C1CC1]

### Comparative Example 3

N-(4-fluorobenzyl) cyclopropanamine was prepared in an analogous manner described in Example 1, and was isolated as an oil (88%). ¹H NMR (400 MHz, CDCl₃) δ 7.32-7.24 (m, 2H), 6.99 (t, J=8.6 Hz, 2H), 3.80 (s, 2H), 2.13 (dt, J=6.3, 3.0 Hz, 1H), 0.41 (dtd, J=24.1, 6.9, 3.5 Hz, 4H).

N-cyclopropyl-4-fluoro-N-(4-fluorobenzyl)benzenesulfonamide was prepared in an analogous manner described in Example 1, and was isolated as a white powder (67%). ¹H NMR (400 MHz, CDCl₃) δ 7.89-7.80 (m, 2H), 7.36-7.28 (m, 2H), 7.24-7.15 (m, 2H), 7.06-6.95 (m, 2H), 4.34 (s, 2H), 2.05-1.96 (m, 1H), 0.71-0.54 (m, 4H).

### Example 19 Synthesis of 2,3,4,5,6-pentafluoro-N-(4-fluorobenzyl)benzenesulfonamide

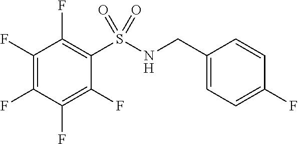 [SMILES: O=S(=O)(NCc1ccc(F)cc1)c1c(F)c(F)c(F)c(F)c1F]

### Comparative Example 4

2,3,4,5,6-pentafluoro-N-(4-fluorobenzyl)benzenesulfonamide was prepared in an analogous manner described in Example 1, and was isolated as a powder (76%). ¹H NMR (400 MHz, CDCl₃) δ 7.29-7.19 (m, 2H), 7.00 (t, J=8.5 Hz, 2H), 5.49 (s, 1H), 4.34 (d, J=4.6 Hz, 2H).

### Example 20 Synthesis of 2,3,4,5,6-pentafluoro-N-(4-fluorophenyl)benzenesulfonamide

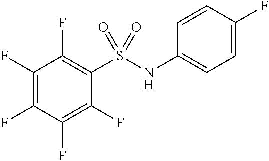 [SMILES: O=S(=O)(Nc1ccc(F)cc1)c1c(F)c(F)c(F)c(F)c1F]

### Comparative Example 5

To a solution of 4-fluoroanaline (0.45 mmol) in pyridine (0.45 mmol) at 0° C. was added pentafluorobenzenesulfonyl chloride (0.495 mmol) in a dropwise manner. The reaction mixture was allowed to gradually warm to room temperature and stirred for 16 hours. The reaction was quenched with water and DCM, and the aqueous phase extracted with DCM (3×). The combined organic phase was washed once with a saturated solution of sodium chloride, dried with MgSO₄, and concentrated in vacuo. The crude sample was absorbed onto a small amount of silica and purified using flash chromatography using an EtOAc:Hex gradient. 2,3,4,5,6-pentafluoro-N-(4-fluorophenyl)benzenesulfonamide was isolated as a white solid (48%). ¹H NMR (400 MHz, CDCl₃) δ 7.27-7.15 (m, 2H), 7.10-6.98 (m, 2H).

### Example 21 Synthesis of N-cyclopropyl-2,3,4,5,6-pentafluorobenzenesulfonamide

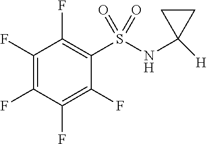 [SMILES: O=S(=O)(NC1CC1)c1c(F)c(F)c(F)c(F)c1F]

### Comparative Example 6

N-cyclopropyl-2,3,4,5,6-pentafluorobenzenesulfonamide was prepared in an analogous manner described in Example 1, and was isolated as a white powder (77%). ¹H NMR (400 MHz, CDCl₃) δ 5.47 (s, 1H), 2.47-2.39 (m, 1H), 0.82-0.70 (m, 4H).

### Example 22 Synthesis of 2,3,4,5,6-pentafluoro-N-(1-phenylcyclopropyl)benzenesulfonamide

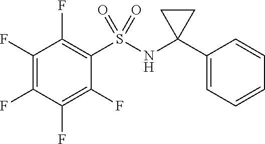 [SMILES: O=S(=O)(NC1(c2ccccc2)CC1)c1c(F)c(F)c(F)c(F)c1F]

### Comparative Example 7

2,3,4,5,6-pentafluoro-N-(1-phenylcyclopropyl)benzenesulfonamide was prepared in an analogous manner described in Example 1, and was isolated as a beige powder (61%). ¹H NMR (400 MHz, CDCl₃) δ 7.30-7.24 (m, 2H), 7.19-7.01 (m, 3H), 6.16 (s, 1H), 1.51 (dd, J=7.32, 7.04 Hz, 2H), 1.20 (dd, J=7.36, 7.04 Hz, 2H).

### Example 23 Synthesis of N-cyclopropyl-2,3,4,5,6-pentafluoro-N-(3-fluoro-4-methoxybenzyl)benzenesulfonamide

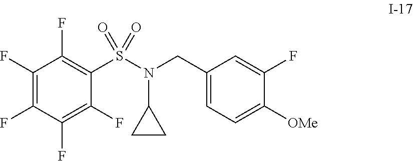 [SMILES: COc1ccc(CN(C2CC2)S(=O)(=O)c2c(F)c(F)c(F)c(F)c2F)cc1F]

N-(3-fluoro-4-methoxybenzyl)cyclopropanamine was prepared in an analogous manner described in Example 1, and was isolated as a beige solid (80%). ¹H NMR (400 MHz, CDCl₃) δ 7.13-7.07 (m, 2H), 6.94 (t, J=8 Hz, 1H), 3.90 (s, 3H), 3.88 (s, 2H), 2.26-2.22 (m, 1H), 0.60-0.55 (m, 4H).

N-cyclopropyl-2,3,4,5,6-pentafluoro-N-(3-fluoro-4-methoxybenzyl)benzenesulfonamide was prepared in an analogous manner described in Example 1, and was isolated as a white powder (48%). ¹H NMR (400 MHz, CDCl₃) δ 7.13-7.09 (m, 2H), 7.08-6.91 (m, 1), 4.47 (s, 2H), 3.89 (s, 3H), 2.38-2.36 (m, 1H), 0.73-0.71 (m, 4H).

### Example 24 Synthesis of Ethyl 1-((2,3,4,5,6-pentafluoro-N-((5-fluoropyridin-2-yl)methyl)phenyl)sulfonamido)cyclopropane-1-carboxylate

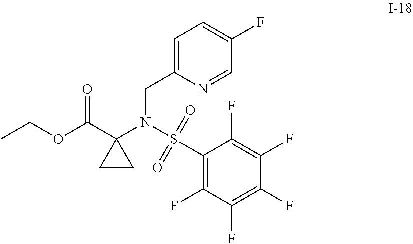 [SMILES: CCOC(=O)C1(N(Cc2ccc(F)cn2)S(=O)(=O)c2c(F)c(F)c(F)c(F)c2F)CC1]

Ethyl 1-(((5-fluoropyridin-2-yl)methyl)amino)cyclopropane-1-carboxylate was prepared in an analogous manner described in Example 3, and was isolated as a colourless oil (66%).

Ethyl 1-((2,3,4,5,6-pentafluoro-N-((5-fluoropyridin-2-yl)methyl)phenyl)sulfonamido)cyclopropane-1-carboxylate was prepared in an analogous manner described in Example 3, and was isolated as a white solid (55%). ¹H NMR (400 MHz, CDCl₃) δ 8.33 (s, 1H), 7.40-7.35 (m, 2H), 4.78 (s, 2H), 4.15-4.04 (m, 2H), 1.52-1.42 (m, 4H), 1.22-1.18 (t, J=7.1 Hz, 1H).

### Example 25 Synthesis of Compound Ethyl 1-((perfluorophenyl)sulfonamido)cyclopropane-1-carboxylate

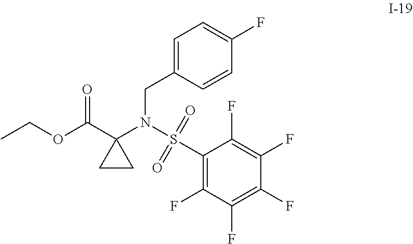 [SMILES: CCOC(=O)C1(N(Cc2ccc(F)cc2)S(=O)(=O)c2c(F)c(F)c(F)c(F)c2F)CC1]

Ethyl 1-((2,3,4,5,6-pentafluoro-N-((5-fluoropyridin-2-yl)methyl)phenyl)sulfonamido)cyclopropane-1-carboxylate was prepared in an analogous manner described in Example 1, and was isolated as a white solid. ¹H NMR (400 MHz, CDCl₃) δ 7.38-7.33 (m, 2H), 7.08-7.02 (m, 2H), 5.05-4.37 (m, 2H), 4.01 (m, 2H), 2.01-0.84 (m, 4H), 1.20, 1.18, 1.17 (t, J=7.1 Hz, 3H).

### Example 26 Synthesis of 4-(((N-cyclopropyl-2,3,4,5,6-pentafluorophenyl)sulfonamido)methyl)benzamide

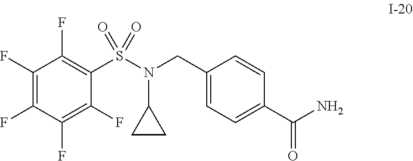 [SMILES: NC(=O)c1ccc(CN(C2CC2)S(=O)(=O)c2c(F)c(F)c(F)c(F)c2F)cc1]

4-(((N-cyclopropyl-2,3,4,5,6-pentafluorophenyl)sulfonamido)methyl)benzamide was prepared in an analogous manner described in Example 1, and was isolated as a white powder (62%). ¹H NMR (400 MHz, Methanol-d4) δ 0.69 (dd, J=5.6, 3.6 Hz, 4H), 2.47 (dq, J=6.9, 4.4, 3.5 Hz, 1H), 4.64 (s, 2H), 7.51 (d, J=8.0 Hz, 2H), 7.89 (d, J=8.2 Hz, 2H).

### Example 27 Synthesis of N-cyclopropyl-2,3,4,5,6-pentafluoro-N-(4-hydroxy-3-methoxybenzyl)benzenesulfonamide

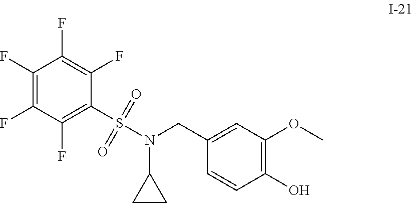 [SMILES: COc1cc(CN(C2CC2)S(=O)(=O)c2c(F)c(F)c(F)c(F)c2F)ccc1O]

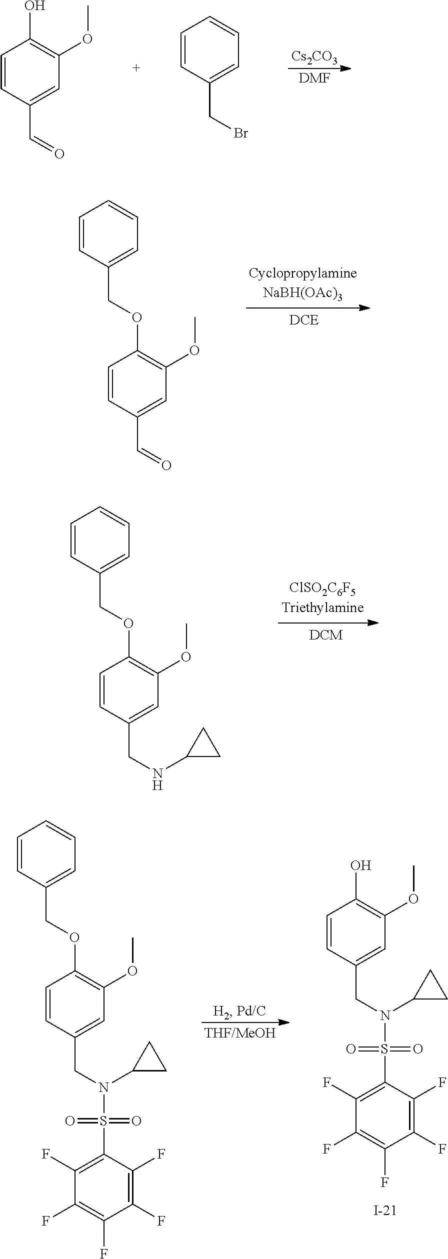

To a solution of vanillin (3.77 mmol) in DMF (0.5 M) was added cesium carbonate (4.15 mmol). The resulting mixture was stirred for 10 minutes under N₂ atmosphere at room temperature. Then, benzyl bromide (4.15 mmol) was added in a dropwise manner and the mixture was stirred 12 hours. The reaction was quenched with water and further diluted with EtOAc. The two layers were partitioned and the organic fraction was washed with a saturated solution of NaCl. The organic layer was dried over MgSO₄ and concentrated in vacuo. The crude sample was absorbed onto a small amount of silica and purified using flash chromatography using a gradient of EtOAc:Hexanes. 4-(benzyloxy)-3-methoxybenzaldehyde was isolated as a light yellow oil (27%). ¹H NMR (400 MHz, CDCl₃) δ 9.86 (s, 1H), 7.47-7.33 (m, 7H), 7.02-7.00 (d, J=7.01 Hz, 1H), 5.27 (s, 2H), 3.97 (s, 3H).

N-(4-(benzyloxy)-3-methoxybenzyl)cyclopropanamine was prepared in an analogous manner described in Example 3, and was isolated as an oil (35%). ¹H NMR (400 MHz, CDCl₃) δ 7.47-7.28 (m, 5H), 6.90-6.78 (m, 3H), 5.16 (s, 2H), 3.92 (s, 3H), 3.79 (s, 2H), 2.18-2.15 (m, 1H), 0.49-0.39 (m, 4H).

N-(4-(benzyloxy)-3-methoxybenzyl)-N-cyclopropyl-2,3,4,5,6-pentafluorobenzenesulfonamide was prepared in an analogous manner described in Example 3, and was isolated as a white solid (24%). ¹H NMR (400 MHz, CDCl₃) δ 7.45-7.28 (m, 5H), 6.94 (s, 1H), 6.83 (s, 1H), 5.14 (s, 2H), 4.48 (s, 2H), 3.90 (s, 3H), 2.39-2.36 (m, 1H), 0.72-0.70 (m, 4H).

To a solution of N-(4-(benzyloxy)-3-methoxybenzyl)-N-cyclopropyl-2,3,4,5,6-pentafluorobenzenesulfonamide (0.1 mmol) in THF (0.06 M) and MeOH (0.12 M) was added Pd/C (5 mg). The resulting mixture was stirred and flushed once with hydrogen, and left stir at room temperature under hydrogen for 2 hours. The reaction mixture was filtered through a pad of celite and concentrated in vacuo. The title compound was isolated using prep HPLC and was lyophilized from water/acetonitrile to afford a free-flowing white powder (90%). ¹H NMR (400 MHz, CDCl₃) δ 6.95-6.82 (m, 3H), 5.65 (s, 1H), 4.49 (s, 2H), 3.93 (s, 3H), 2.41-2.36 (m, 1H), 0.74-0.73 (m, 4H).

### Example 28 Synthesis of N-cyclopropyl-N-(3,5-dimethoxybenzyl)-2,3,4,5,6-pentafluorobenzenesulfonamide

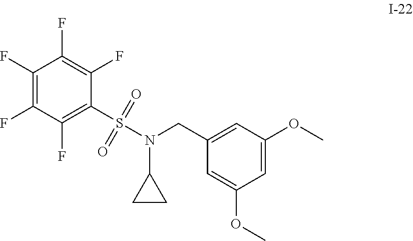 [SMILES: COc1cc(CN(C2CC2)S(=O)(=O)c2c(F)c(F)c(F)c(F)c2F)cc(OC)c1]

N-(3,5-dimethoxybenzyl)cyclopropanamine was prepared in an analogous manner described in Example 3, and was isolated as a white solid (78%). ¹H NMR (400 MHz, Chloroform-d) δ 0.36 (ttd, J=9.6, 7.4, 7.0, 4.8 Hz, 4H), 2.11 (dp, J=7.0, 3.8 Hz, 1H), 4.25 (s, 2H), 6.30 (t, J=2.4 Hz, 1H), 6.43 (d, J=2.3 Hz, 2H).

N-cyclopropyl-N-(3,5-dimethoxybenzyl)-2,3,4,5,6-pentafluorobenzenesulfonamide was prepared in an analogous manner described in Example 3, and was isolated as a white powder (32%). ¹H NMR (400 MHz, Chloroform-d) δ 0.66-0.82 (m, 4H), 2.43 (dtt, J=7.5, 4.7, 2.4 Hz, 1H), 4.46 (s, 2H), 6.36 (q, J=1.8, 1.3 Hz, 1H), 6.48 (d, J=2.1 Hz, 2H).

### Example 29 Synthesis of N-(4-cyanobenzyl)-N-cyclopropyl-2,3,4,5,6-pentafluorobenzenesulfonamide

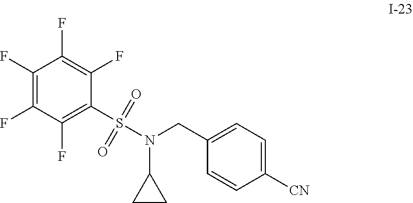 [SMILES: N#Cc1ccc(CN(C2CC2)S(=O)(=O)c2c(F)c(F)c(F)c(F)c2F)cc1]

N-(4-cyanobenzyl)-N-cyclopropyl-2,3,4,5,6-pentafluorobenzenesulfonamide was prepared in an analogous manner described in Example 1, and was isolated as a white powder (68%). ¹H NMR (400 MHz, CDCl₃) δ 7.66-7.64 (m, 2H), 7.51-7.49 (m, 2H), 4.59 (s, 2H), 2.39-2.34 (m, 1H), 0.71-0.66 (m, 4H).

### Example 30 Synthesis of N-cyclopropyl-2,3,4,5,6-pentafluoro-N-(4(methylsulfonyl)benzyl)benzenesulfonamide

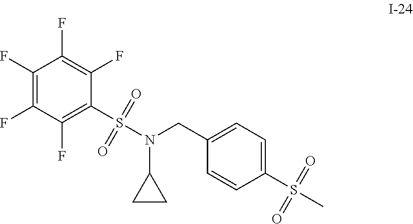 [SMILES: CS(=O)(=O)c1ccc(CN(C2CC2)S(=O)(=O)c2c(F)c(F)c(F)c(F)c2F)cc1]

N-(4-(methylsulfonyl)benzyl)cyclopropanamine was prepared in an analogous manner described in Example 3, and was isolated as a white solid (72%). ¹H NMR (400 MHz, Chloroform-d) δ 0.43 (dd, 2H), 0.46 (dd, 2H), 2.16 (tt, J=6.8, 3.7 Hz, 1H), 3.05 (s, 3H), 3.94 (s, 2H), 7.53 (d, J=8.1 Hz, 2H), 7.89 (d, J=8.2, 3.9 Hz, 2H).

N-cyclopropyl-2,3,4,5,6-pentafluoro-N-(4(methylsulfonyl)benzyl)benzenesulfonamide was prepared in an analogous manner described in Example 3, and was isolated as a white powder (58%).¹H NMR (400 MHz, Acetonitrile-d₃) δ 0.69-0.74 (m, 4H), 2.51 (p, J=5.9 Hz, 1H), 3.09 (s, 2H), 4.68 (s, 1H), 7.64 (d, J=8.3 Hz, 1H), 7.94 (d, J=8.3 Hz, 1H).

### Example 31 Synthesis of N-cyclopropyl-2,3,4,5,6-pentafluoro-N-(4-(trifluoromethyl)benzyl)benzenesulfonamide

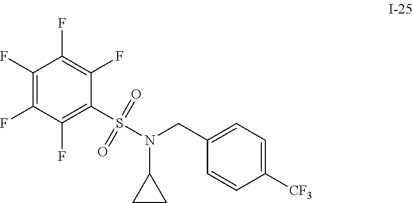 [SMILES: O=S(=O)(c1c(F)c(F)c(F)c(F)c1F)N(Cc1ccc(C(F)(F)F)cc1)C1CC1]

N-(4-(trifluoromethyl)benzyl)cyclopropanamine was prepared in an analogous manner described in Example 3, and was isolated as a white solid (88%). ¹H NMR (400 MHz, Chloroform-d) δ 0.41 (d, 2H), 0.46 (d, 2H), 2.12-2.21 (m, 1H), 3.91 (s, 2H), 7.45 (d, 2H), 7.59 (d, J=8.1 Hz, 2H).

N-cyclopropyl-2,3,4,5,6-pentafluoro-N-(4-(trifluoromethyl)benzyl)benzenesulfonamide was prepared in an analogous manner described in Example 1, and was isolated as a white powder (65%). ¹H NMR (400 MHz, Chloroform-d) δ 0.72 (s, 2H), 0.74 (d, J=1.7 Hz, 2H), 2.42 (p, J=5.4 Hz, 1H), 4.63 (s, 2H), 7.53 (d, J=8.0 Hz, 2H), 7.64 (d, J=8.1 Hz, 2H).

### Example 32 Synthesis of 4-(((N-cyclopropyl-2,3,4,5,6-pentafluorophenyl)sulfonamido)methyl)benzoic Acid

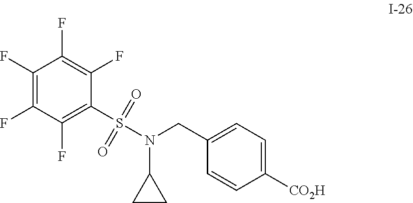 [SMILES: O=C(O)c1ccc(CN(C2CC2)S(=O)(=O)c2c(F)c(F)c(F)c(F)c2F)cc1]

Benzyl 4-(((N-cyclopropyl-2,3,4,5,6-pentafluorophenyl)sulfonamido)methyl)benzoate was prepared in an analogous manner described in Example 1 using benzyl 4-(bromomethyl)benzoate as R—X, and was isolated as a white solid (68%). ¹H NMR (400 MHz, Chloroform-d) δ 0.71 (d, J=5.4 Hz, 4H), 2.39 (p, J=5.4 Hz, 1H), 4.62 (s, 2H), 5.40 (s, 2H), 7.35-7.50 (m, 7H), 8.03-8.13 (m, 2H).

An oven dried flask was charged with benzyl 4-(((N-cyclopropyl-2,3,4,5,6-pentafluorophenyl)sulfonamido)methyl)benzoate and purged with N₂. A 2:1 mixture of THF/MeOH (0.25 M) and 10% Pd/C was then subsequently added. A balloon of H₂ was then introduced and the progress of the reaction was monitored by TLC. Upon completion, the reaction was filtered through celite and was purified by prep HPLC. 4-(((N-cyclopropyl-2,3,4,5,6-pentafluorophenyl)sulfonamido)methyl)benzoic acid was isolated as free-flowing white powder (92%). ¹H NMR (400 MHz, Methanol-d₄) δ 0.70 (dq, J=6.9, 2.5 Hz, 4H), 2.48 (dt, J=6.0, 2.6 Hz, 1H), 4.65 (s, 2H), 7.46-7.54 (m, 2H), 7.99-8.06 (m, 2H).

### Example 33 Synthesis of Methyl 4-(((N-cyclopropyl-2,3,4,5,6-pentafluorophenyl)sulfonamido)methyl)benzoate

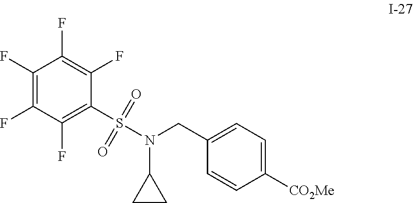 [SMILES: COC(=O)c1ccc(CN(C2CC2)S(=O)(=O)c2c(F)c(F)c(F)c(F)c2F)cc1]

Methyl 4-(((N-cyclopropyl-2,3,4,5,6-pentafluorophenyl)sulfonamido)methyl)benzoate was prepared in an analogous manner described in Example 1, and was isolated as a white powder (70%). ¹H NMR (400 MHz, CDCl₃) δ_{H} 8.02 (d, J=8.4 Hz, 2H), 7.45 (d, J=8.4 Hz, 2H), 4.59 (s, 2H), 3.92 (s, 3H), 2.42-2.32 (m, 1H), 0.69 (d, J=5.4 Hz, 4H) ppm.

### Example 34 Synthesis of N-cyclopropyl-2,3,4,5,6-pentafluoro-N-(4-hydroxybenzyl)benzenesulfonamide

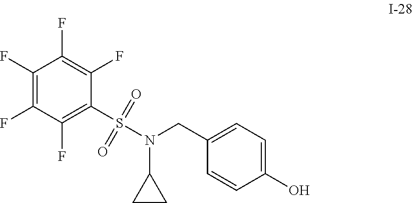 [SMILES: O=S(=O)(c1c(F)c(F)c(F)c(F)c1F)N(Cc1ccc(O)cc1)C1CC1]

1-(benzyloxy)-4-(bromomethyl)benzene was prepare according to literature. (Reference: Org. Lett., Vol. 14, No. 21, 2012)

N-(4-(benzyloxy)benzyl)-N-cyclopropyl-2,3,4,5,6-pentafluorobenzenesulfonamide was prepared in an analogous manner described in Example 1 using 1-(benzyloxy)-4-(bromomethyl)benzene as a compound of Formula J. N-(4-(benzyloxy)benzyl)-N-cyclopropyl-2,3,4,5,6-pentafluorobenzenesulfonamide (0.046 mmol) and Pd/C (3 mg) were dissolved in THF (0.06 M) and MeOH (0.12 M). A balloon of H₂ was then introduced and the progress of the reaction was monitored by TLC. Upon completion, the reaction was filtered through celite and was purified by prep HPLC. N-cyclopropyl-2,3,4,5,6-pentafluoro-N-(4-hydroxybenzypenzenesulfonamide was isolated as a free-flowing white powder (72%). ¹H NMR (400 MHz, CDCl₃) δ 7.28-7.26 (m, 2H), 6.83-6.80 (m, 2H), 4.83 (s, 1H), 4.50 (s, 2H), 2.40-2.35 (m, 1H), 0.74-0.72 (m, 4H).

### Example 35 Synthesis of N-cyclopropyl-2,3,4,5,6-pentafluoro-N-(4-hydroxy-3,5-dimethoxybenzyl)benzenesulfonamide

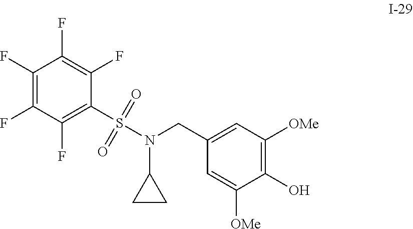 [SMILES: COc1cc(CN(C2CC2)S(=O)(=O)c2c(F)c(F)c(F)c(F)c2F)cc(OC)c1O]

4-(benzyloxy)-3,5-dimethoxybenzaldehyde was prepared from 4-hydroxy-3,5-dimethoxybenzaldehyde and isolated as a light yellow oil (92%). ¹H NMR (400 MHz, CDCl₃) δ 9.85 (s, 1H), 7.49-7.47 (m, 2H), 7.37-7.27 (m, 3H), 7.11 (s, 2H), 5.13 (s, 2H), 3.88 (s, 6H).

N-(4-(benzyloxy)-3,5-dimethoxybenzyl)cyclopropanamine was prepared in an analogous manner described in Example 3, and was isolated as an oil (72%). ¹H NMR (400 MHz, CDCl₃) δ 7.52-7.50 (m, 2H), 7.38-7.29 (m, 3H), 6.56 (s, 2H), 5.01 (s, 2H), 3.85 (s, 3H), 3.81 (s, 2H), 2.23-2.18 (m, 1H), 0.51-0.42 (m, 4H).

N-(4-(benzyloxy)-3,5-dimethoxybenzyl)-N-cyclopropyl-2,3,4,5,6-pentafluorobenzenesulfonamide was prepared in an analogous manner described in Example 3, and was isolated as a an oil (24%). ¹H NMR (400 MHz, CDCl₃) δ 7.45-7.28 (m, 5H), 6.94 (s, 1H), 6.83 (s, 1H), 5.14 (s, 2H), 4.48 (s, 2H), 3.90 (s, 3H), 2.39-2.36 (m, 1H), 0.72-0.70 (m, 4H).

To a solution of N-(4-(benzyloxy)-3,5-dimethoxybenzyl)-N-cyclopropyl-2,3,4,5,6-pentafluorobenzenesulfonamide (0.22 mmol) in THF (0.06 M) and MeOH (0.12 M) was added Pd/C (12 mg). A balloon of H2 was then introduced and the reaction mixture was stirred at room temperature for 2 hours. The reaction mixture was filtered through a pad of celite and purified by prep HPLC. N-cyclopropyl-2,3,4,5,6-pentafluoro-N-(4-hydroxy-3,5-dimethoxybenzyl)benzenesulfonamide was isolated a white powder (88%). ¹H NMR (400 MHz, CDCl₃) δ 6.61 (s, 2H), 5.55 (s, 1H), 4.48 (s, 2H), 3.90 (s, 3H), 2.41-2.38 (m, 1H), 0.74-0.72 (m, 4H).

### Example 36 Synthesis of N-cyclopropyl-2,3,4,5,6-pentafluoro-N-(4-(4-methylpiperazin-1-yl)benzyl)benzenesulfonamide

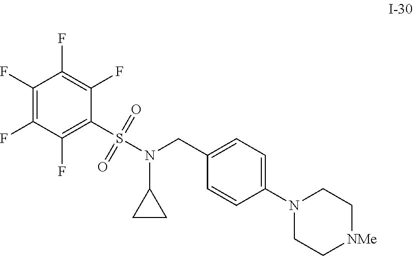 [SMILES: CN1CCN(c2ccc(CN(C3CC3)S(=O)(=O)c3c(F)c(F)c(F)c(F)c3F)cc2)CC1]

N-(4-(4-methylpiperazin-1-yl)benzyl)cyclopropanamine was prepared in an analogous manner described in Example 3, and was isolated as a dark orange oil (54%). ¹H NMR (400 MHz, Chloroform-d) δ 0.43 (dd, 4H), 2.16 (tt, J=6.5, 4.2 Hz, 1H), 2.36 (s, 3H), 2.59 (t, 4H), 3.20 (t, 4H), 3.77 (s, 2H), 6.90 (d, 2H), 7.22 (d, 2H).

N-cyclopropyl-2,3,4,5,6-pentafluoro-N-(4-(4-methylpiperazin-1-yl)benzyl)benzenesulfonamide was prepared in an analogous manner described in Example 3, and was isolated as a white powder (94%). ¹H NMR (400 MHz, Chloroform-d) δ 0.74 (dd, 4H), 2.35-2.49 (m, 1H), 2.56 (s, 3H), 2.83 (t, J=5.1 Hz, 4H), 3.32 (t, 4H), 4.48 (s, 2H), 6.87 (d, 2H), 7.26 (d, 2H).

### Example 37 Synthesis of N-cyclopropyl-2,3,4,5,6-pentafluoro-N-(3-phenylprop-2-yn-1-yl)benzenesulfonamide

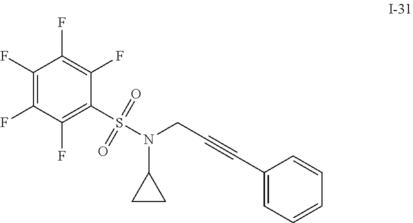 [SMILES: O=S(=O)(c1c(F)c(F)c(F)c(F)c1F)N(CC#Cc1ccccc1)C1CC1]

N-(3-phenylprop-2-yn-1-yl)cyclopropanamine was prepared in an analogous manner described in Example 3, and was isolated as a light yellow oil (55%). ¹H NMR (400 MHz, CDCl₃) δ 7.46-7.44 (m, 2H), 7.34-7.31 (m, 3H), 3.7 (s, 2H), 2.48-2.43 (m, 1H), 0.54-0.43 (m, 4H).

N-cyclopropyl-2,3,4,5,6-pentafluoro-N-(3-phenylprop-2-yn-1-yl)benzenesulfonamide was prepared in an analogous manner described in Example 3, and was isolated as a white powder (46%). ¹H NMR (400 MHz, CDCl₃) δ 7.38-7.29 (m, 3H), 7.23-7.20 (m, 2H), 4.47 (s, 2H), 2.64-2.59 (m, 1H), 1.09-0.91 (m, 4H).

### Example 38 Synthesis of N-cyclopropyl-2,3,4,5,6-pentafluoro-N-(pyridin-2-ylmethyl)benzenesulfonamide

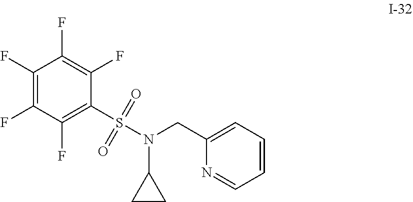 [SMILES: O=S(=O)(c1c(F)c(F)c(F)c(F)c1F)N(Cc1ccccn1)C1CC1]

N-cyclopropyl-2,3,4,5,6-pentafluoro-N-(pyridin-2-ylmethyl)benzenesulfonamide was prepared in an analogous manner described in Example 1, and was isolated as a white powder (76%). ¹H NMR (400 MHz, CDCl₃) δ_{H} 8.37 (dd, J=4.9, 1.8 Hz, 1H), 7.68 (td, J=7.7, 1.8 Hz, 1H), 7.39 (d, J=7.8 Hz, 1H), 7.18 (dd, J=7.6, 4.9 Hz, 1H), 4.69 (s, 2H), 2.56 (tt, J=7.0, 3.7 Hz, 1H), 0.84-0.79 (m, 2H), 0.74-0.67 (m, 2H) ppm.

### Example 39 Synthesis of N-cyclopropyl-2,3,4,5,6-pentafluoro-N-(3-phenylprop-2-yn-1-yl)benzenesulfonamide

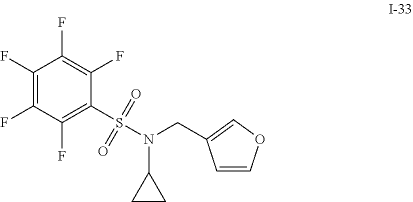 [SMILES: O=S(=O)(c1c(F)c(F)c(F)c(F)c1F)N(Cc1ccoc1)C1CC1]

N-(furan-3-ylmethyl)cyclopropanamine was prepared in an analogous manner described in Example 3, and was isolated as a an oil (80%). ¹H NMR (400 MHz, CDCl₃) δ 7.40-7.39 (t, J=1.8 Hz, 1H), 7.37 (m, 1H), 6.40 (s, 1H), 3.72 (s, 2H), 2.21-2.17 (m, 1H), 0.49-0.39 (m, 4H).

N-cyclopropyl-2,3,4,5,6-pentafluoro-N-(3-phenylprop-2-yn-1-yl)benzenesulfonamide was prepared in an analogous manner described in Example 3, and was isolated as a white powder (46%). ¹H NMR (400 MHz, CDCl₃) δ 7.44 (s, 1H), 7.40 (s, 1H), 6.44 (s, 1H), 4.45 (s, 2H), 2.48-2.43 (m, 1H), 0.82-0.80 (m, 4H).

### Example 40 Synthesis of N-cyclopropyl-2,3,4,5,6-pentafluoro-N-(3-morpholinobenzyl)benzenesulfonamide

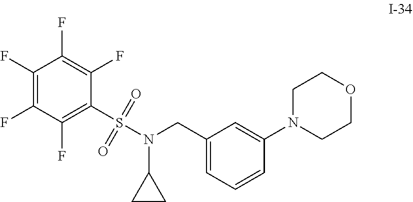 [SMILES: O=S(=O)(c1c(F)c(F)c(F)c(F)c1F)N(Cc1cccc(N2CCOCC2)c1)C1CC1]

N-(3-morpholinobenzyl)cyclopropanamine was prepared in an analogous manner described in Example 3, and was isolated as a bright yellow oil (66%). ¹H NMR (400 MHz, Chloroform-d) δ 0.43 (dd, 2H), 0.46 (dd, 2H), 2.19 (ddd, J=8.5, 6.6, 3.7 Hz, 1H), 3.19 (t, 4H), 3.83 (s, 2H), 3.88 (t, 4H), 6.84 (td, 2H), 6.91 (s, 1H), 7.26 (t, 1H).

N-cyclopropyl-2,3,4,5,6-pentafluoro-N-(3-morpholinobenzyl)benzenesulfonamide was prepared in an analogous manner described in Example 3, and was isolated as a white powder (45%). ¹H NMR (400 MHz, Acetonitrile-d3) δ 0.70 (d, 2H), 0.72 (q, 2H), 2.45-2.59 (m, 1H), 3.13 (tt, 4H), 3.80 (tt, 4H), 4.53 (s, 2H), 6.81-6.95 (m, 3H), 7.25 (t, J=7.9 Hz, 1H).

### Example 41 Synthesis of N-cyclopropyl-N-(2,4-difluorobenzyl)-2,3,4,5,6-pentafluorobenzenesulfonamide

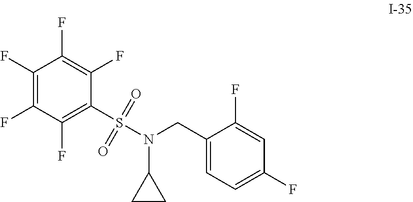 [SMILES: O=S(=O)(c1c(F)c(F)c(F)c(F)c1F)N(Cc1ccc(F)cc1F)C1CC1]

N-cyclopropyl-N-(2,4-difluorobenzyl)-2,3,4,5,6-pentafluorobenzenesulfonamide was prepared in an analogous manner described in Example 1, and was isolated as a white powder (68%). ¹H NMR (400 MHz, CDCl₃) δ 7.66-7.64 (td, J=8.6, 6.4 Hz, 1H), 6.96-6.91 (m, 1H), 6.86-6.80 (ddd, J=10.1, 8.7, 2.6 Hz 1H), 4.61 (s, 2H), 2.41-2.35 (m, 1H), 0.74-0.73 (m, 4H).

### Example 42 Synthesis of N-cyclopropyl-2,3,4,5,6-pentafluoro-N-(2,4,6-trifluorobenzyl)benzenesulfonamide

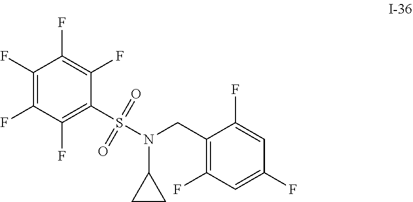 [SMILES: O=S(=O)(c1c(F)c(F)c(F)c(F)c1F)N(Cc1c(F)cc(F)cc1F)C1CC1]

N-cyclopropyl-2,3,4,5,6-pentafluoro-N-(2,4,6-trifluorobenzyl)benzenesulfonamide was prepared in an analogous manner described in Example 1, and was isolated as a white powder (21%). ¹H NMR (400 MHz, CDCl₃) δ 6.73-6.69 (dd, J=8.7, 7.5 Hz, 2H), 4.64 (s, 2H), 2.22-2.17 (m, 1H), 0.77-0.67 (m, 4H).

### Example 43 Synthesis of N-cyclopropyl-N-((3,5-difluoropyridin-2-yl)methyl)-2,3,4,5,6-pentafluorobenzenesulfonamide

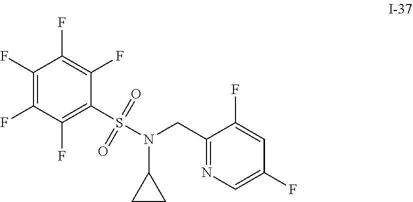 [SMILES: O=S(=O)(c1c(F)c(F)c(F)c(F)c1F)N(Cc1ncc(F)cc1F)C1CC1]

N-((3,5-difluoropyridin-2-yl)methyl)cyclopropanamine was prepared in an analogous manner described in Example 3, and was isolated as an oil (47%). ¹H NMR (400 MHz, CDCl₃) δ 8.29-8.28 (d, J=2.4 Hz, 1H), 7.18-7.13 (ddd, J=9.1, 8.2, 2.4 Hz, 1H), 4.00 (d, J=1.3 Hz, 2H), 2.15-2.10 (m, 1H), 0.45-0.36 (m, 4H).

N-cyclopropyl-N-((3,5-difluoropyridin-2-yl)methyl)-2,3,4,5,6-pentafluorobenzenesulfonamide was prepared in an analogous manner described in Example 3, and was isolated as a white powder (74%). ¹H NMR (400 MHz, CDCl₃) δ 8.13 (d, J=2.3 Hz, 1H), 7.25-7.21 (ddd, J=9.2, 8.0, 2.4 Hz, 1H), 4.82 (s, 2H), 2.60-2.55 (m, 1H), 0.92-0.87 (m, 2H), 0.78-0.73 (m, 2H).

### Example 44 Synthesis of N-((4′-amino-[1,1′-biphenyl]-2-yl)methyl)-N-cyclopropyl-2,3,4,5,6-pentafluorobenzenesulfonamide

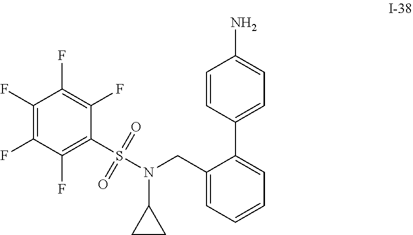 [SMILES: Nc1ccc(-c2ccccc2CN(C2CC2)S(=O)(=O)c2c(F)c(F)c(F)c(F)c2F)cc1]

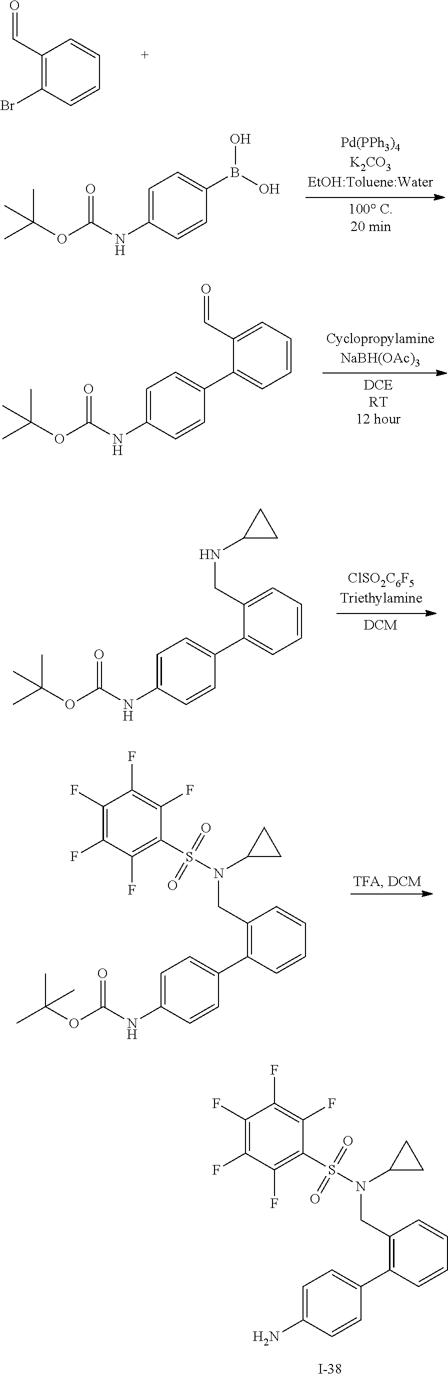

A solution of 2-bromobenzaldehyde (1.08 mmol), 4-boc-aminophenylboronic acid (1.19 mmol) and potassium carbonate (3.24 mmol) in a 0.1 M solvent mixture of ethanol:toluene:water (9:3:1) was stirred at room temperature for 5 minutes under nitrogen. Tetrakis(triphenylphosphine)pallaidum(0) (0.108 mmol) was then added and the mixture was stirred under microwave irradiation at 100° C. for 20 minutes. The mixture was then filtered through a pad of celite and concentrated in vacuo. The crude sample was redissolved in DCM and water, and transferred to a separatory funnel. The two layers were partitioned and the aqueous layer was extracted with DCM (3×). The collected organic layers were then washed once with saturated NaCl solution, dried over MgSO₄ and concentrated in vacuo. The crude sample was absorbed onto a small amount of silica and purified using flash chromatography using a Hexane:EtOAc gradient. tert-butyl (2′-formyl-[1,1′-biphenyl]-4-yl)carbamate was isolated as an oil (86%). ¹H NMR (400 MHz, CDCl₃) δ 10.01 (s, 1H), 8.03-8.01 (dd, J=7.8, 1.4 Hz, 1H), 7.65-7.61 (td, J=7.5, 1.5 Hz, 1H), 7.54-7.42 (m, 4H), 7.33-7.31 (d, J=8.5 Hz, 2H), 6.88 (s, 1H), 1.55 (s, 9H).

Tert-butyl (2′-((cyclopropylamino)methyl)-[1,1′-biphenyl]-4-yl)carbamate was prepared in an analogous manner described in Example 3, and was isolated as an oil (15%). ¹H NMR (400 MHz, CDCl₃) δ 7.45-7.25 (m, 8H), 6.66 (s, 1H), 3.81 (s, 2H), 2.05-2.02 (m, 1H), 1.57 (s, 9H), 0.39-0.30 (m, 4H).

Tert-butyl (2′-(((N-cyclopropyl-2,3,4,5,6-pentafluorophenyl)sulfonamido)methyl)-[1,1′-biphenyl]-4-yl)carbamate was prepared in an analogous manner described in Example 3, and was isolated as a light yellow solid (66%). ¹H NMR (400 MHz, CDCl₃) δ 8.13 (d, J=2.3 Hz, 1H), 7.64-7.61 (m, 1H), 7.45-7.32 (m, 4H), 7.24-7.20 (m, 4H), 6.57 (s, 1H), 2.22-2.20 (m, 1H), 1.56 (s, 9H), 0.55-0.51 (m, 4H).

To a solution of tert-butyl (2′-(((N-cyclopropyl-2,3,4,5,6-pentafluorophenyl)sulfonamido)methyl)-[1,1′-biphenyl]-4-yl)carbamate was (0.065 mmol) in DCM (0.06 M) was added with TFA (0.065 mmol). The resulting mixture was stirred at room temperature for 3 hours. The mixture was then concentrated in vacuo. The crude product was then diluted with EtOAc and washed three times with saturated sodium bicarbonate solution. The collected organic layers were dried over MgSO₄ and concentrated in vacuo. N-((4′-amino-[1,1′-biphenyl]-2-yl)methyl)-N-cyclopropyl-2,3,4,5,6-pentafluorobenzenesulfonamide was purified by prep HPLC affording a white powder (86%). ¹H NMR (400 MHz, CDCl₃) δ 7.60-7.59 (d, J=7.3 Hz, 1H), 7.38-7.32 (m, 2H), 7.24-7.22 (d, J=7.3 Hz, 1H), 7.08-7.05 (d, J=8.4 Hz, 2H), 6.76-6.74 (d, J=8.4 Hz, 2H), 4.58 (s, 2H), 3.79 (br, 2H), 2.23-2.21 (m, 1H), 0.57-0.51 (m, 4H).

### Example 45 Synthesis of N-((3′-amino-[1,1′-biphenyl]-4-yl)methyl)-N-cyclopropyl-2,3,4,5,6-pentafluorobenzenesulfonamide

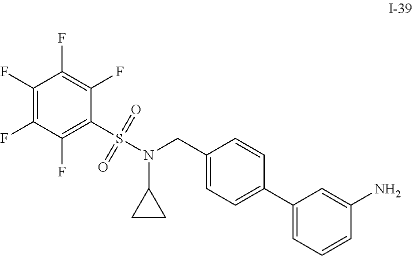 [SMILES: Nc1cccc(-c2ccc(CN(C3CC3)S(=O)(=O)c3c(F)c(F)c(F)c(F)c3F)cc2)c1]

A solution of 4-bromobenzaldehyde (0.654 mmol), 3-[(tert-butoxycarbonyl)amino]phenylboronic acid (0.719 mmol) and potassium carbonate (1.96 mmol) in a 0.1 M solvent mixture of ethanol:toluene:water (9:3:1) was stirred at room temperature for 5 minutes under N₂. Tetrakis(triphenylphosphine)pallaidum(0) (0.0654 mmol) was then added and the mixture was stirred under microwave irradiation at 100° C. for 20 minutes. The mixture was then filtered through a pad of celite and concentrated in vacuo. The crude sample was redissolved in DCM and water, and transferred to a separatory funnel. The two layers were partitioned and the aqueous layer was extracted with DCM (3×). The collected organic layers were then washed once with saturated NaCl solution, dried over MgSO₄ and concentrated in vacuo. The crude sample was absorbed onto a small amount of silica and purified using flash chromatography using a Hexane:EtOAc gradient. tert-butyl (4′-formyl-[1,1′-biphenyl]-3-yl)carbamate was isolated as an oil (58%). ¹H NMR (400 MHz, CDCl₃) δ 10.07 (s, 1H), 7.94 (d, J=2.6 Hz, 2H), 7.81 (s, 1H), 7.77 (d, J=2.9 Hz, 2H), 7.43-7.29 (m, 3H), 6.76 (s, 1H), 1.56 (s, 9H).

Tert-butyl (4′-((cyclopropylamino)methyl)-[1,1′-biphenyl]-3-yl)carbamate was prepared in an analogous manner described in Example 3, and was isolated as an oil (47%). ¹H NMR (400 MHz, CDCl₃) δ 7.64 (s, 1H), 7.58-7.56 (d, J=8.2 Hz, 2H), 7.41-7.39 (d, J=8.0 Hz, 2H), 7.34-7.27 (m, 3H) 6.57 (s, 1H), 3.91 (s, 2H), 2.24-2.19 (m, 1H), 1.56 (s, 9H), 0.52-0.43 (m, 4H).

Tert-butyl (4′-(((N-cyclopropyl-2,3,4,5,6-pentafluorophenyl)sulfonamido)methyl)-[1,1′-biphenyl]-3-yl)carbamate was prepared in an analogous manner described in Example 3, and was isolated as a beige solid (51%). ¹H NMR (400 MHz, CDCl₃) δ 7.69 (s, 1H), 7.59-7.57 (d, J=8.2 Hz, 2H), 7.45-7.43 (d, J=8.2 Hz, 2H), 7.40-7.26 (m, 3H), 6.58 (s, 1H), 4.61 (s, 2H), 2.47-2.42 (m, 1H), 1.56 (s, 9H), 0.80-0.72 (m, 4H).

To a solution of tert-butyl (4′-(((N-cyclopropyl-2,3,4,5,6-pentafluorophenyl)sulfonamido)methyl)-[1,1′-biphenyl]-3-yl)carbamate (0.075 mmol) in DCM (0.1 M) was added with TFA (0.065 mmol). The resulting mixture was stirred at room temperature for 3 hours. The mixture was then concentrated in vacuo. The crude product was then diluted with EtOAc and washed three times with saturated sodium bicarbonate solution. The collected organic layers were dried over MgSO₄ and concentrated in vacuo. N-((3′-amino-[1,1′-biphenyl]-4-yl)methyl)-N-cyclopropyl-2,3,4,5,6-pentafluorobenzenesulfonamide was purified by prep HPLC affording a white powder (80%). ¹H NMR (400 MHz, CDCl₃) δ 7.56-7.54 (d, J=8.2 Hz, 2H), 7.44-7.42 (d, J=8.2 Hz, 2H), 7.28-7.24 (t, J=7.8 Hz, 1H), 7.01-6.98 (m, 1H), 6.92-6.91 (m, 1H), 6.73-6.70 (m, 1H), 4.61 (s, 2H), 2.48-2.45 (m, 1H), 0.79-0.75 (m, 4H).

### Example 46 Synthesis of N-cyclopropyl-2,3,4,5,6-pentafluoro-N-(2-fluorobenzyl)benzenesulfonamide

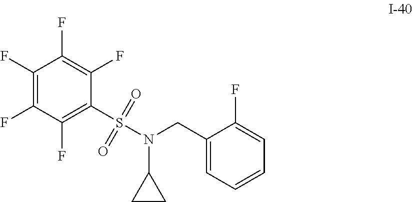 [SMILES: O=S(=O)(c1c(F)c(F)c(F)c(F)c1F)N(Cc1ccccc1F)C1CC1]

N-cyclopropyl-2,3,4,5,6-pentafluoro-N-(2-fluorobenzyl)benzenesulfonamide was prepared in an analogous manner described in Example 1, and was isolated as a white powder (63%). ¹H NMR (400 MHz, CDCl₃) δ 7.53-7.49 (td, J=7.6, 1.8 Hz, 1H), 7.35-7.29 (m, 1H), 7.20-7.16 (dd, 1H), 7.07-7.03 (ddd, 9.7, 8.2, 1.2 Hz, 1H), 4.65 (s, 2H), 2.45-2.40 (m, 1H), 0.79-0.70 (m, 4H).

### Example 47 Synthesis of Benzyl 4-(((N-cyclopropyl-2,3,4,5,6-pentafluorophenyl)sulfonamido)methyl)benzoate

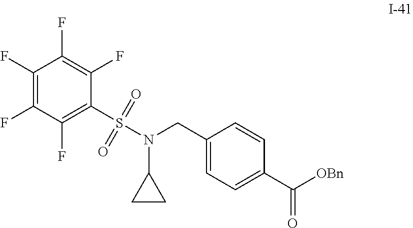 [SMILES: O=C(OCc1ccccc1)c1ccc(CN(C2CC2)S(=O)(=O)c2c(F)c(F)c(F)c(F)c2F)cc1]

Benzyl 4-(((N-cyclopropyl-2,3,4,5,6-pentafluorophenyl)sulfonamido)methyl)benzoate was prepared in an analogous manner described in Example 1, and was isolated as white powder (77%).¹H NMR (400 MHz, Chloroform-d) δ 0.71 (d, J=5.4 Hz, 4H), 2.39 (p, J=5.4 Hz, 1H), 4.62 (s, 2H), 5.40 (s, 2H), 7.35-7.50 (m, 7H), 8.03-8.13 (m, 2H).

### Example 48 Synthesis of tert-butyl 5-(((N-cyclopropyl-2,3,4,5,6-pentafluorophenyl)sulfonamido)methyl)-1H-indole-1-carboxylate

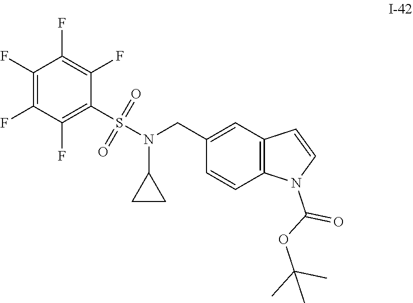 [SMILES: CC(C)(C)OC(=O)n1ccc2cc(CN(C3CC3)S(=O)(=O)c3c(F)c(F)c(F)c(F)c3F)ccc21]

A round bottom flask was charged with indole-5-carboxaldehyde (2.07 mmol), di-tert-butyl dicarbonate (3.1 mmol), 4-(dimethylamino)pyridine (0.207 mmol) and DCM (0.1 M). Et₃N (6.2 mmol) was then added dropwise and the mixture was left to stir at room temperature. The progress of the reaction was monitored by TLC and upon complete conversion of the starting material the reaction was quenched with a saturated solution of NH₄Cl. The two layers were partitioned and the aqueous layer was extracted with DCM (3×). Combined organic fractions were washed with a saturated solution of NaCl, dried over MgSO₄ and concentrated in vacuo to provide tert-butyl 5-formyl-1-1H-indole-1-carboxylate of sufficient purity to proceed to the next step. ¹H NMR (400 MHz, Chloroform-d) δ 1.71 (s, 10H), 6.71 (d, J=3.8 Hz, 1H), 7.71 (d, J=3.8 Hz, 1H), 7.88 (dd, J=8.6, 1.6 Hz, 1H), 8.12 (d, J=1.6 Hz, 1H), 8.31 (d, J=8.6 Hz, 1H), 10.08 (s, 1H).

tert-butyl 5-((cyclopropylamino)methyl)-1H-indole-1-carboxylate was prepared in an analogous manner described in Example 3, and was isolated as a white solid (70%). ¹H NMR (400 MHz, Chloroform-d) δ 0.29-0.46 (m, 4H), 1.58-1.70 (m, 9H), 2.13 (tdd, J=10.0, 5.2, 3.1 Hz, 1H), 3.87 (d, J=7.1 Hz, 2H), 6.48 (t, J=3.7 Hz, 1H), 7.21 (dd, J=8.4, 1.9 Hz, 1H), 7.43-7.48 (m, 1H), 7.54 (q, J=3.7 Hz, 1H), 8.05 (d, J=8.1 Hz, 1H).

tert-butyl 5-(((N-cyclopropyl-2,3,4,5,6-pentafluorophenyl)sulfonamido)methyl)-1H-indole-1-carboxylate was prepared in an analogous manner described in Example 3, and was isolated as a white solid (51%). ¹H NMR (400 MHz, Chloroform-d) δ 0.74 (ddt, J=8.9, 4.5, 2.5 Hz, 4H), 1.71 (s, 9H), 2.39-2.46 (m, 1H), 4.67 (s, 2H), 6.57 (dd, J=3.7, 0.8 Hz, 1H), 7.33 (dd, J=8.6, 1.8 Hz, 1H), 7.57-7.60 (m, 1H), 7.64 (d, J=3.7 Hz, 1H), 8.11 (d, J=8.5 Hz, 1H).

### Example 49 Synthesis of N-cyclopropyl-2,3,4,5,6-pentafluoro-N-((3-(trifluoromethyl)pyridin-2-yl)methyl)benzenesulfonamide

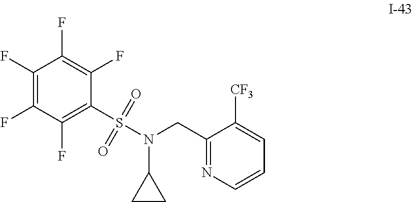 [SMILES: O=S(=O)(c1c(F)c(F)c(F)c(F)c1F)N(Cc1ncccc1C(F)(F)F)C1CC1]

N-((3-(trifluoromethyl)pyridin-2-yl)methyl)cyclopropanamine was prepared in an analogous manner described in Example 3, and was isolated as an oil (88%). ¹H NMR (400 MHz, CDCl₃) δ 8.80-8.78 (d, J=4.2 Hz, 1H), 8.05-8.04 (d, J=1.6 Hz, 1H), 7.44-7.41 (dd, J=8.0, 4.8 Hz, 1H). 4.07 (s, 2H), 2.17-2.12 (m, 1H), 0.43-0.30 (m, 4H).

N-cyclopropyl-2,3,4,5,6-pentafluoro-N-((3-(trifluoromethyl)pyridin-2-yl)methyl)benzenesulfonamide was prepared in an analogous manner described in Example 3, and was isolated as white powder (55%).

### Example 50 Synthesis of N-cyclopropyl-2,3,4,5,6-pentafluoro-N-((3-(trifluoromethyl)pyridin-2-yl)methyl)benzenesulfonamide

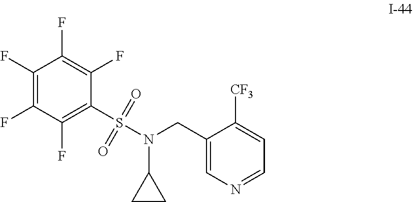 [SMILES: O=S(=O)(c1c(F)c(F)c(F)c(F)c1F)N(Cc1cnccc1C(F)(F)F)C1CC1]

N-((4-(trifluoromethyl)pyridin-3-yl)methyl)cyclopropanamine was prepared in an analogous manner described in Example 3, and was isolated as an oil (88%). ¹H NMR (400 MHz, CDCl₃) δ 8.88 (s, 1H), 8.80-8.79 (m, 1H), 7.58-7.57 (d, J=5.0, 1H). 4.02 (s, 2H), 2.18-2.13 (m, 1H), 0.45-0.41 (m, 2H), 0.32-0.29 (m, 2H).

N-cyclopropyl-2,3,4,5,6-pentafluoro-N-((3-(trifluoromethyl)pyridin-2-yl)methyl)benzenesulfonamide was prepared in an analogous manner described in Example 3, and was isolated as a white powder (40%). ¹H NMR (400 MHz, CDCl₃) δ 8.89 (s, 1H), 8.80-8.79 (d, J=5.1 Hz, 1H, 1H), 7.68-7.66 (d, J=5.1 Hz, 1H), 4.81 (s, 2H), 2.52-2.47 (m, 1H), 0.68-0.67 (m, 4H).

### Example 51 Synthesis of N-cyclobutyl-2,3,4,5,6-pentafluoro-N-(2-fluorobenzyl)benzenesulfonamide

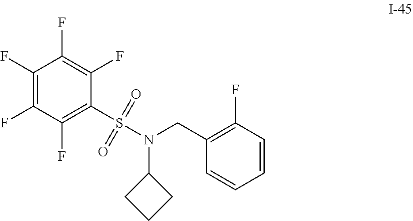 [SMILES: O=S(=O)(c1c(F)c(F)c(F)c(F)c1F)N(Cc1ccccc1F)C1CCC1]

N-cyclobutyl-2,3,4,5,6-pentafluoro-N-(2-fluorobenzyl)benzenesulfonamide was prepared in an analogous manner described in Example 1, and was isolated as a white powder (80%). ¹H NMR (400 MHz, Chloroform-d) δ 1.51-1.68 (m, 2H), 2.06 (dd, 3H), 2.11 (dd, 2H), 4.45 (ddd, J=17.4, 9.7, 7.7 Hz, 1H), 4.68 (s, 2H), 7.01 (ddd, J=10.3, 8.2, 1.2 Hz, 1H), 7.17 (td, J=7.6, 1.2 Hz, 1H), 7.28 (tdd, J=7.4, 5.2, 1.8 Hz, 1H), 7.51 (td, J=7.7, 1.7 Hz, 1H).

### Example 52 Synthesis of Compound I-46

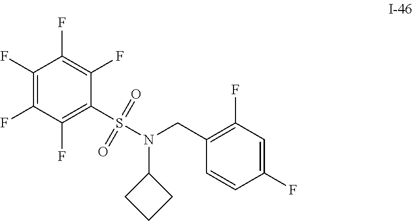 [SMILES: O=S(=O)(c1c(F)c(F)c(F)c(F)c1F)N(Cc1ccc(F)cc1F)C1CCC1]

N-cyclobutyl-N-(2,4-difluorobenzyl)-2,3,4,5,6-pentafluorobenzenesulfonamide was prepared in an analogous manner described in Example 1, and was isolated as a white powder (68%). ¹H NMR (400 MHz, Chloroform-d) δ 1.63 (dd, 2H), 2.08 (dd, 4H), 4.40 (t, 1H), 4.64 (s, 2H), 6.81 (ddd, J=10.8, 8.6, 2.6 Hz, 1H), 6.95 (dd, J=8.3, 2.5 Hz, 1H), 7.53 (td, J=8.6, 6.2 Hz, 1H).

### Example 53 Synthesis of N-cyclobutyl-2,3,4,5,6-pentafluoro-N-(2,4,6-trifluorobenzyl)benzenesulfonamide

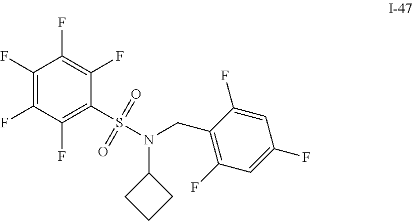 [SMILES: O=S(=O)(c1c(F)c(F)c(F)c(F)c1F)N(Cc1c(F)cc(F)cc1F)C1CCC1]

N-cyclobutyl-2,3,4,5,6-pentafluoro-N-(2,4,6-trifluorobenzyl)benzenesulfonamide was prepared in an analogous manner described in Example 1, and was isolated as a white powder (95%). ¹H NMR (400 MHz, Chloroform-d) δ 1.59-1.71 (m, 2H), 2.08 (dtt, J=12.2, 7.4, 2.4 Hz, 2H), 2.24 (pd, J=9.8, 2.8 Hz, 2H), 4.31 (ddd, J=17.5, 10.0, 7.6 Hz, 1H), 4.66 (s, 2H), 6.66 (t, J=8.3 Hz, 2H).

### Example 54 Synthesis of N-cyclopropyl-2,3,4,5,6-pentafluoro-N-(2-fluorobenzyl)benzenesulfonamide

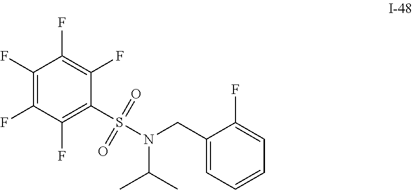 [SMILES: CC(C)N(Cc1ccccc1F)S(=O)(=O)c1c(F)c(F)c(F)c(F)c1F]

N-cyclopropyl-2,3,4,5,6-pentafluoro-N-(2-fluorobenzypenzenesulfonamide was prepared in an analogous manner described in Example 1, and was isolated as a white powder (48%). ¹H NMR (400 MHz, CDCl₃) δ 7.62-7.57 (td, 7.7, 1.8 Hz, 1H), 7.31-7.25 (m, 1H), 7.18-7.14 (td, J=7.6, 1.2 Hz, 1H), 7.01-6.96 (ddd, J=9.6, 8.2, 1.2 Hz, 1H), 4.60 (s, 2H), 4.42-4.32 (m, 1H), 1.17-1.15 (m, 4H).

### Example 55 Synthesis of N-(2,4-difluorobenzyl)-2,3,4,5,6-pentafluoro-N-isopropylbenzenesulfonamide

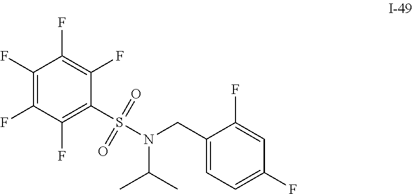 [SMILES: CC(C)N(Cc1ccc(F)cc1F)S(=O)(=O)c1c(F)c(F)c(F)c(F)c1F]

N-(2,4-difluorobenzyl)-2,3,4,5,6-pentafluoro-N-isopropylbenzenesulfonamide was prepared in an analogous manner described in Example 1, and was isolated as a white powder (76%). ¹H NMR (400 MHz, Chloroform-d) δ 1.13 (d, J=6.8 Hz, 6H), 4.32 (p, J=6.8 Hz, 1H), 4.57 (s, 2H), 6.77 (ddd, J=10.9, 8.7, 2.6 Hz, 1H), 6.92 (td, J=8.3, 2.4 Hz, 1H), 7.62 (td, J=8.7, 6.3 Hz, 1H).

### Example 56 Synthesis of 2,3,4,5,6-pentafluoro-N-isopropyl-N-(2,4,6-trifluorobenzyl)benzenesulfonamide

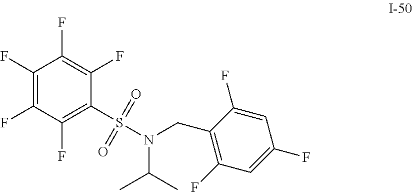 [SMILES: CC(C)N(Cc1c(F)cc(F)cc1F)S(=O)(=O)c1c(F)c(F)c(F)c(F)c1F]

2,3,4,5,6-pentafluoro-N-isopropyl-N-(2,4,6-trifluorobenzyl)benzenesulfonamide was prepared in an analogous manner described in Example 1, and was isolated as a white powder (82%). ¹H NMR (400 MHz, Chloroform-d) δ 1.20 (d, J=6.8 Hz, 6H), 4.31 (p, J=6.8 Hz, 1H), 4.60 (s, 2H), 6.64 (t, J=8.2 Hz, 2H).

### Example 57 Synthesis of 2,3,4,5,6-pentafluoro-N-(4-fluorobenzyl)-N-(tetrahydrofuran-3-yl)benzenesulfonamide

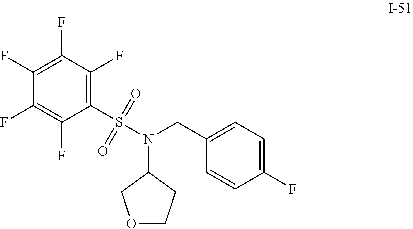 [SMILES: O=S(=O)(c1c(F)c(F)c(F)c(F)c1F)N(Cc1ccc(F)cc1)C1CCOC1]

2,3,4,5,6-pentafluoro-N-(4-fluorobenzyl)-N-(tetrahydrofuran-3-yl)benzenesulfonamide was prepared in an analogous manner described in Example 1, and was isolated as a white powder (71%). ¹H NMR (400 MHz, CDCl₃) δ 7.40-7.36 (m, 2H), 7.05-6.99 (m, 2H), 4.58-4.49 (d, 1H), 3.76-3.65 (m, 2H), 3.62-3.56 (q, 1H), 2.26-2.18 (m, 1H), 1.89-1.80 (m, 1H).

### Example 58 Synthesis of 2,3,4,5,6-pentafluoro-N-(4-fluorobenzyl)-N-(pyridin-4-ylmethypenzenesulfonamide

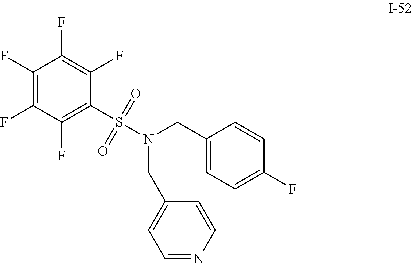 [SMILES: O=S(=O)(c1c(F)c(F)c(F)c(F)c1F)N(Cc1ccncc1)Cc1ccc(F)cc1]

2,3,4,5,6-pentafluoro-N-(4-fluorobenzyl)-N-(pyridin-4-ylmethAtienzenesulfonamide was prepared in an analogous manner described in Example 4 using 4-(bromomethyl)pyridine as R—X, and was isolated as a beige solid (38%). ¹H NMR (400 MHz, Chloroform-d) δ 4.49 (s, 2H), 4.54 (s, 2H), 6.90-6.98 (m, 2H), 7.07-7.16 (m, 4H), 8.49-8.56 (m, 2H).

### Example 59 Synthesis of 2,3,4,5,6-pentafluoro-N-(4-fluorobenzyl)-N-phenylbenzenesulfonamide

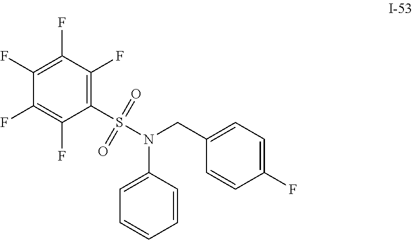 [SMILES: O=S(=O)(c1c(F)c(F)c(F)c(F)c1F)N(Cc1ccc(F)cc1)c1ccccc1]

2,3,4,5,6-pentafluoro-N-(4-fluorobenzyl)-N-phenylbenzenesulfonamide was prepared in an analogous manner described in Example 1, and was isolated as a white powder (38%). ¹H NMR (400 MHz, CDCl₃) δ 7.33-7.29 (m, 3H), 7.24-7.20 (m, 2H), 7.08-7.05 (m, 2H), 7.00-6.95 (m, 2H) 4.96 (s, 1H), 3.76-3.65 (m, 2H), 3.62-3.56 (q, 1H), 2.26-2.18 (m, 1H), 1.89-1.80 (m, 1H).

### Example 60 Synthesis of N-(4-fluorobenzyl)-N-((perfluorophenyl)sulfonyl)pivalamide

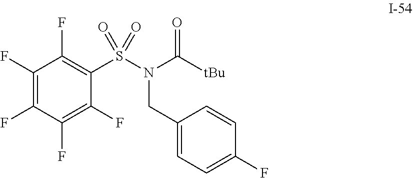 [SMILES: CC(C)(C)C(=O)N(Cc1ccc(F)cc1)S(=O)(=O)c1c(F)c(F)c(F)c(F)c1F]

N-(4-fluorobenzyl)-N-((perfluorophenyl)sulfonyl)pivalamide was prepared in an analogous manner as Example 4 and isolated as a white powder (70 mg, 55%). ¹H NMR (400 MHz, CDCl₃) δ 7.37-7.34 (dd, J=8.5, 5.2 Hz, 2H), 7.12-7.07 (t, J=8.6 Hz, 2H), 5.15 (s, 2H), 1.20 (s, 9H).

### Example 61 Synthesis of N-cyclobutyl-2,3,4,5,6-pentafluoro-N-(4-fluorobenzyl)benzenesulfonamide

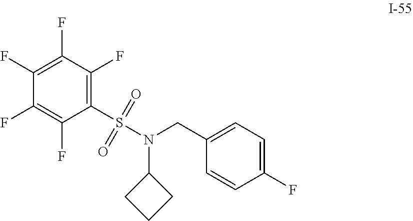 [SMILES: O=S(=O)(c1c(F)c(F)c(F)c(F)c1F)N(Cc1ccc(F)cc1)C1CCC1]

N-cyclobutyl-2,3,4,5,6-pentafluoro-N-(4-fluorobenzyl)benzenesulfonamide was prepared in an analogous manner described in Example 1, and was isolated as a white powder (79%).¹H NMR (400 MHz, Chloroform-d) δ 1.50-1.68 (m, 2H), 2.04 (dd, 2H), 2.09 (dd, 2H), 4.38 (ddd, J=17.3, 9.7, 7.8 Hz, 1H), 4.59 (s, 2H), 7.04 (dd, 2H), 7.34 (dd, 2H).

### Example 62 Synthesis of Methyl 4-(((N-cyclopentyl-2,3,4,5,6-pentafluorophenyl)sulfonamido)methyl)benzoate

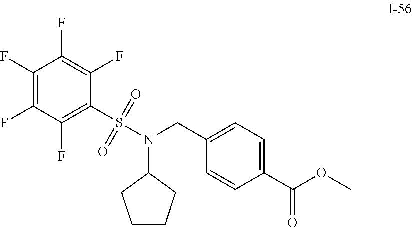 [SMILES: COC(=O)c1ccc(CN(C2CCCC2)S(=O)(=O)c2c(F)c(F)c(F)c(F)c2F)cc1]

Methyl 4-(((N-cyclopentyl-2,3,4,5,6-pentafluorophenyl)sulfonamido)methyl)benzoate was prepared in an analogous manner described in Example 1, and was isolated as a white powder (48%). ¹H NMR (400 MHz, CDCl₃) δ_{H} 17.98 (d, J=8.3 Hz, 2H), 7.43 (d, J=8.4 Hz, 2H), 4.58 (s, 2H), 4.43-4.26 (m, 1H), 3.91 (s, 3H), 1.82-1.67 (m, 2H), 1.63-1.30 (m, 6H) ppm.

### Example 63 Synthesis of N-cyclopropyl-2,3,5,6-tetrafluoro-N-(4-fluorobenzyl)benzenesulfonamide

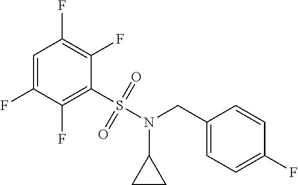 [SMILES: O=S(=O)(c1c(F)c(F)cc(F)c1F)N(Cc1ccc(F)cc1)C1CC1]

### Comparative Example 8

A microwave vial was charged with 1,2,4,5-tetrafluorobenzene (6.66 mmol) and chlorosulfonic acid (30 mmol). The vessel was capped and flushed with nitrogen. The reaction mixture was then stirred at 120° C. for 3 hours. The reaction was quenched with ice-cold 1M HCl, and extracted three times with EtOAc. Combined organic fractions were washed with a saturated solution of NaCl, dried over MgSO4 and concentrated in vacuo. 2,3,5,6-tetrafluorobenzenesulfonic acid was isolated as a brown oil and used directly in the next step. ¹H NMR (400 MHz, CDCl₃) δ 7.57-7.49 (tt, J=9.0, 7.0 Hz, 1H)

N-cyclopropyl-2,3,5,6-tetrafluoro-N-(4-fluorobenzyl)benzenesulfonamide was prepared in an analogous manner as Example 63, and was isolated as a white powder (47%). ¹H NMR (400 MHz, CDCl₃) δ 7.42-7.25 (m, 3H), 7.07-7.02 (t, J=8.6 Hz, 1H), 4.55 (s, 2H), 2.42-2.38 (m, 1H), 0.72-0.71 (m, 4H).

### Example 64 Synthesis of N-(tert-butyl)-2,3,4,5,6-pentafluoro-N-(4-fluorobenzyl)benzenesulfonamide

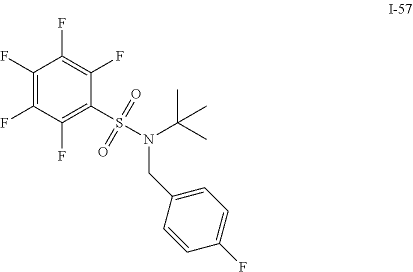 [SMILES: CC(C)(C)N(Cc1ccc(F)cc1)S(=O)(=O)c1c(F)c(F)c(F)c(F)c1F]

N-(tert-butyl)-2,3,4,5,6-pentafluoro-N-(4-fluorobenzyl) benzenesulfonamide was prepared in an analogous manner described in Example 1, and was isolated as a white powder (41%). ¹H NMR (400 MHz, CDCl₃) δ 7.48-7.44 (dd, 8.6, 5.3 Hz, 1H), 7.10-7.06 (t, J=8.6 Hz, 1H), 4.80 (s, 2H), 1.34 (m, 4H).

### Example 65 Synthesis of 2,3,4,5,6-pentafluoro-N-(4-fluorobenzyl)-N-methylbenzenesulfonamide

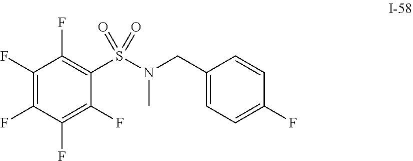 [SMILES: CN(Cc1ccc(F)cc1)S(=O)(=O)c1c(F)c(F)c(F)c(F)c1F]

2,3,4,5,6-pentafluoro-N-(4-fluorobenzyl)-N-methylbenzenesulfonamide was prepared in an analogous manner described in Example 4, and was isolated as a white solid (55%). ¹H NMR (400 MHz, CDCl₃) δ 7.34 (dd, J=8.5, 5.6 Hz, 2H), 7.07 (t, J=8.5 Hz, 2H), 4.39 (s, 2H), 2.85 (s, 3H).

### C. In Vitro Cell Viability Studies

### Example 66 Exemplary Compounds Cytotoxicity Analysis

The anti-cancer efficacy of exemplary compounds of the application were assessed in vitro against different cancer cell lines. Cell viability was examined following treatment at various concentrations of inhibitor (0.097656-50 μM) using a CellTiter-Blue® cell viability assay. 1×10⁴ cells/well were plated in 96-well assay plates in culture medium. All cells were grown in DMEM, IMDM and RPMI-1640 supplemented with 10% fetal bovine serum (FBS). In some instances, FBS was removed for periods ranging from 16-24 hours, and re-introduced with test compound addition. After 24 hrs, test compounds and vehicle controls were added to appropriate wells such that the final volume was 100 μl in each well. The cells were cultured for the desired test exposure period (72 hours) at 37° C. and 5% CO₂. The assay plates were removed from 3° C. incubator and 20 μl/well of CellTiter-Blue® Reagent was added. The plates were incubated using standard cell culture conditions for 1-4 hours. Afterwards, the plates were shaken for 10 seconds and florescence was recorded at 560/590 nm using a Cytation 3 spectrophotometer. IC₅₀ values were determined using non-linear regression analysis with GraphPad Prism 6.0 (GraphPad Software Inc.).

Exemplary compounds of the present application showed IC₅₀ values in the range of low micromolar to nanomolar against cancer cells, such as MV4-11 and MOLM13. It is noted that the IC₅₀values for healthy cells, such as MRC9 and HACAT, were typically in the double digit micromolar range, indicating a substantial therapeutic window. Of note, comparative examples 4 and 5, where R¹═H, are less potent than the corresponding parent I-1 analogue; Comparative examples 2, 3, and 8 that lack a PFBS substituent are inactive in AML cells, demonstrating the need for this group.

Table 1 summarizes IC₅₀ values of compounds in cancerous and healthy cell lines following the protocol in Example 66.

TABLE 1
IC₅₀ values of compounds against major acute myeloid
leukemia cell lines (AML) (MV4-11 and MOLM13), healthy
human lung cells (MRC9), healthy keratinocyte cells
(HaCaT) and primary human fibroblast cells (PHF).

| | AML | | Non-Cancerous | | |
|---|---|---|---|---|---|
| | IC₅₀ | IC₅₀ | IC₅₀ | IC₅₀ | IC₅₀ |
| | MV4-11 | MOLM13 | MRC9 | HaCaT | PHF |
| Compound | (μM) | (μM) | (μM) | (μM) | (μM) |
| I-1 | 0.47 | 1.5 | 25 | 12.5 | 3.4 |
| I-2 | 0.85 | 1.6 | 7.7 | | 2.9 |
| I-3 | 0.59 | 9.3 | 14.4 | | |
| I-4 | 2.09 | 5.1 | 25 | | |
| I-5 | 0.7 | 2.0 | | | |
| I-6 | 1.3 | 1.3 | 17.5 | | |
| I-7 | 1.37 | 3.5 | 16.4 | | 2.7 |
| I-8 | 0.93 | 0.73 | | 17.4 | |
| I-9 | 1.83 | 1.62 | | | |
| I-10 | 0.974 | 9.83 | | | |
| I-11 | 1.7 | 3.37 | 9 | | |
| I-12 | 1.35 | 3.07 | 18 | | 5.0 |
| I-13 | 1.7 | 1.1 | 25 | | 5.5 |
| I-16 | 1.2 | 3.5 | 25 | | 7.2 |
| I-17 | 0.79 | 0.63 | 16.2 | | |
| I-18 | 2.8 | 6.2 | | | |
| I-20 | 1.05 | 1.7 | | | 4.9 |
| I-22 | 4.7 | 4.5 | | | |
| I-23 | 0.99 | 2.58 | 7.8 | | 5.5 |
| I-24 | 1.15 | 1.35 | | | |
| I-25 | 0.95 | 1.05 | | | 3 |
| I-26 | 1.2 | 1.9 | | | 6.5 |
| I-27 | 2.0 | 0.9 | | | |
| I-29 | 1.94 | 3.96 | 9.7 | | |
| I-31 | 1.53 | 5.2 | 15.5 | | |
| I-32 | 1.5 | 1.3 | | | |
| I-33 | 3.55 | 3.86 | 22.1 | | |
| I-34 | 0.96 | 7.95 | 14.1 | | |
| I-35 | 1.7 | 3.96 | 13.5 | | |
| I-36 | 1.52 | 6.81 | 26.0 | | 8.1 |
| I-37 | 1.69 | 2.89 | 22 | | 8.7 |
| I-38 | 1.43 | 8.51 | 13.1 | | |
| I-39 | 1.52 | 14.9 | 15.7 | | |
| I-40 | 0.33 | 3.2 | | | |
| I-41 | 1.0 | 3.2 | | | |
| I-43 | 1.67 | 4.1 | 8.4 | | |
| I-45 | 2.27 | 3.9 | 14.2 | | |
| I-47 | 1.14 | 8.4 | 15.7 | | 3.7 |
| I-48 | 1.19 | 4.11 | 15 | | 3.0 |
| I-49 | 1.01 | 4.2 | 16.7 | | 4.5 |
| I-51 | 4.8 | 3.37 | | | |
| I-52 | 4.1 | 3.5 | | | |
| I-53 | 1.4 | 1.5 | | | |
| I-54 | 0.7 | 1.7 | | | 3.0 |
| I-55 | 0.43 | 0.73 | | | 2.5 |
| I-58 | 0.44 | 3.1 | | | |
| Comparative | 25 | 25 | | | |
| Example 2 | | | | | |
| Comparative | >25 | >25 | >25 | >25 | |
| Example 3 | | | | | |
| Comparative | 3.1 | 4.0 | 16.8 | | |
| Example 4 | | | | | |
| Comparative | 4.4 | 17.6 | | | |
| Example 5 | | | | | |
| Comparative | 6.4 | 7.0 | 19.1 | | |
| Example 6 | | | | | |
| Comparative | 31 | | | | |
| Example 7 | | | | | |
| Comparative | >12.5 | >25 | | | |
| Example 8 | | | | | |

### Example 67 Anti-Cancer Activity of Exemplary Compound I-1

In addition to cell lines tested in Example 66, exemplary compound I-1 was tested for its efficacy against select glioblastoma, medulloblastoma, chronic myelogenous leukemia (CML) and acute myeloid leukemia (AML) using the protocol outlined in Example 66.

Table 2 presents the IC₅₀ values of exemplary compound I-1 against major glioblastoma cell lines.

TABLE 2
IC₅₀ values of I-1 against major glioblastoma cell lines (A-
172, LN-229, LN-18, U118MG, and U87MG), with Tamoxifen control.

| | IC₅₀ | IC50 | IC₅₀ | IC₅₀ | IC₅₀ |
|---|---|---|---|---|---|
| | A-172 | LN-229 | LN-18 | U118MG | U87MG |
| Compound | (μM) | (μM) | (μM) | (μM) | (μM) |
| I-1 | 7.16 | 3.03 | 4.92 | 10.33 | 2.87 |
| Tamoxifen | 16.18 | 13.38 | 13.64 | 13.99 | 15.06 |

Table 3 presents the IC₅₀ values of select exemplary compounds, I-1 and I-7, against major medulloblastoma cell lines.

TABLE 3
IC₅₀ values of select compounds against major medulloblastoma
cell lines (D425, D458 and ATCC3034).

| | IC₅₀ | IC₅₀ | IC₅₀ |
|---|---|---|---|
| | D425 | D458 | ATCC3034 |
| Compound | (μM) | (μM) | (μM) |
| I-1 | 0.8 | 2.2 | 0.8 |
| I-7 | 1.4 | 2.0 | 0.3 |

Table 4 presents the IC₅₀ values of exemplary compound I-1 against major AML and CML cell lines.

TABLE 4
IC₅₀ values of compound I-1 against major AML (MOLM13, MOLM14, MV4-11,
PL21 and OCI-AML3) and CML (AR230 and AR230R) cell lines.

| | AML | | | | | CML | |
|---|---|---|---|---|---|---|---|
| | IC₅₀ | IC₅₀ | IC₅₀ | IC₅₀ | IC₅₀ | IC₅₀ | IC₅₀ |
| | MOLM13 | MOLM14 | MV4-11 | PL-21 | OCI-AML3 | AR230 | AR230R |
| Compound | (μM) | (μM) | (μM) | (μM) | (μM) | (μM) | (μM) |
| I-1 | 2.5 | 1.7-2.5 | 0.6 | 3.5 | 5.6 | 4.1 | 1.5 |

### D. Pharmacokinetics Absorption, Distribution, Metabolism and Excretion (ADME) Studies

### Example 68 Exemplary Compound I-1 Metabolic Stability to Glutathione as Assessed Via ¹⁹F NMR-based Studies

The stability of I-1 towards reaction with glutathione was determined through ¹⁹F NMR experiments. Compounds were prepared at a final concentration of 100 μM in 100 mM HEPES, pH 7.4, 100 μM 5-fluorotryptophan, 10 mM L-glutathione, 10% D₂O (in blank samples, an equivalent volume of HEPES solution was added), 40% DMSO and 1% DMSO. All samples were incubated at 37° C. 1D ¹⁹F NMR experiments were recorded at 37° C. on a 600 MHz spectrometer with an H(F)CN room temperature probe (number of transients=800) (scan width, 150 ppm). 5-Fluorotryptophan served as an internal reference to normalize peak intensity and was innocuous in the reaction. The data was processed and analyzed using MestreNova 10.0 software.

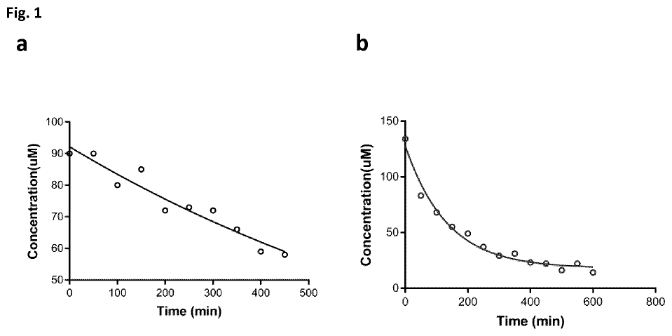

Compound I-1 has an in vitro half-life (T_{1/2}) of longer than 700 minutes. (FIG. 1a) This relatively long half-life in the presence of glutathione indicates stability of I-1 to glutathione. In contrast, T_{1/2} of Batabulin (T138067), a sulfonamide compound disclosed in U.S. Pat. No. 6,482,860B1 and structurally similar to exemplary compound I-1 where the p-fluorobenzyl group in I-1 is replaced by a p-fluorophenyl group, is 93 minutes. (FIG. 1b) The greater than 7-fold increase in T_{1/2} for Compound I-1 as compared to Batabulin suggests that I-1 structural differences from Batabulin, such as its benzyl substituent and/or N-alkyl substituent at the R¹ position, may contribute to the overall stability of the molecule.

### Example 69 Exemplary Compounds Metabolic Stability to Glutathione as Assessed Via ¹⁹F NMR-Based Studies

The stability of the test compounds to glutathione was assessed by examining the production of free fluoride ions via ¹⁹F NMR. For each sample, 100 μM compound was incubated in either the presence of 10 mM glutathione in 100 mM HEPES pH 7.4, 100 μM 5-fluorotryptophan, 10% (v/v) D₂O, 5% (v/v) DMSO. The samples were incubated at 25° C. for 4 hours and 1D ¹⁹F NMR spectra was recorded. The peak intensity of the free fluoride (−119.31 ppm) was determined for each spectra as compared to the intensity of the reference (5-flurotryptophan, −124.73 ppm). The concentration of free fluoride was assessed and normalized to samples containing known concentrations of sodium fluoride. Additionally, negative controls samples containing all the reaction components, excluding glutathione were also run in parallel to confirm the production of fluoride was a solely a consequence of the presence of glutathione.

Table 5 presents the percent free fluoride produced after 4 hours of incubation of compounds and glutathione, as assessed through protocol described in Example 69

TABLE 5
Percent Reactivity of the Compounds with Glutathione
after 4 hours of incubation as analyzed by ¹⁹F NMR.

| Compound | % Fluoride |
|---|---|
| I-3 | 13.3 |
| I-31 | 0.0 |
| I-36 | 7.5 |
| I-40 | 14.7 |
| I46 | 9.3 |
| I-47 | 23.6 |
| I-55 | 13.2 |
| Comparative Example 8 | 6.4 |

Based on these results, select compounds of this application exhibit superior stability to glutathione as compared to Batabulin.

### Example 70 Metabolic Stability of Exemplary Compound I-1 in Pooled Male Mouse Liver S9 Fractions

The metabolic stability of exemplary compound I-1 was further characterized in pooled male mouse live S9 fractions. The reaction mixture was constituted with 100 mM phosphate buffer, ultra-pure H₂O, 5 mM MgCₗ₂ solution, 10 mM NADPH solution and 1 mg/mL S9 fraction. This mixture was then pre-warmed at 37° C. for 5 minutes. The reaction was started with the addition of the test compound (1-1 or Verapamil control) to a final concentration of 2 μM. Aliquots of 50 μL were taken from the reaction solution at 0, 15, 30, 45 and 60 minutes. The aliquoted reaction solutions were stopped by the addition of a mixture of cold methanol and IS (100 nM alprazolam, 200 nM imipramine, 200 nM labetalol and 2 μM ketoprofen). The samples were then centrifuged at 3220 g for 40 minutes. Afterwards, aliquots of 90 μL of the supernatant for each sample was mixed with 90 μL of ultra-pure water and subjected to liquid chromatography tandem mass spectrometry (LC-MS/MS) analysis.

The T_{1/2} was determined by the linear regression of the natural logarithm of the remaining percentage of the parent drug vs. incubation time curve. The slope value (k) of the curve was then substituted into the following equation to determine the T_{1/2}:

$$in vitro {T}_{1 / 2} = - ( \frac{0.693}{k} )$$

The in vitro intrinsic clearance (in vitro CLint, in μL/min/mg protein) was determined by the following equation:

$$in vitro C {L}_{i n t} = ( \frac{0.693}{{T}_{\frac{1}{2}}} ) * ( \frac{volume of incubation}{amount of proteins} )$$

The column used was a Phenomenex Gemini-NX 3μ C18 (2.0×50 mm) with preguard column, with a mobile phase consisting of 0.1% formic acid in acetonitrile (solvent A) and 0.1% formic acid in water (solvent B) at room temperature. Injection volume was 10 μL. MS analysis was carried out on an API 4000 instrument from AB Inc (Canada) with an ESI interface.

Table 6 and 7 present subsequent results of procedure described in Example 70.

TABLE 6
Metabolic stability of I-1 and Verapamil control
in male mouse liver S9 fractions with NADPH.

| Compound | T_{1/2} (min) | CLᵢₙₜ (μL/min/mg protein) |
|---|---|---|
| I-1 | 13910.73 | 0.05 |
| Verapamil | 29.33 | 23.63 |

TABLE 7
Metabolic stability of I-1 and Verapamil control in male mouse
liver S9 fractions, comparison with and without NADPH.

| | | Remaining Percentage (%) | | | | |
|---|---|---|---|---|---|---|
| Compound ID | Assay Format | 0 min | 15 min | 30 min | 45 min | 60 min |
| Verapamil | With NADPH | 100.00 | 62.22 | 42.45 | 30.17 | 24.42 |
| | Without NADPH | 100.00 | 94.90 | 101.94 | 98.79 | 95.87 |
| I-1 | With NADPH | 100.00 | 96.00 | 98.38 | 97.36 | 98.93 |
| | Without NADPH | 100.00 | 98.65 | 102.27 | 95.52 | 104.97 |

The T_{1/2} of I-1 was determined to be 13910.73 minutes, indicating the favourable metabolic stability of this compound, as compared to Verapamil control which had a T_{1/2} of 29.33 minutes.

### Example 71 Intrinsic Clearance of Exemplary Compounds I-1 and I-7 in Mouse Hepatocyte

Intrinsic clearance studies were conducted with compounds I-1 and I-7 in mouse hepatocytes. A stock of 100 μM test compound was prepared by diluting the 10 mM I-1 test compound in DMSO with a solution of 50% acetonitrile and 50% water. In a 96-well non-coated plate, 198 μL of hepatocytes was pipetted, and the plate was placed in the incubator on an orbital shaker to allow the hepatocytes to warm for 10 minutes. To this solution 2 μL of the 100 μM I-1 was added to start the reaction, and the plate was placed on an orbital shaker. At time points of 0, 15, 30, 60, 90 and 120 minutes, the aliquots were mixed with a solution of acetonitrile and internal standard (100 nM alprazolam, 200 nM labetalol, and 2 μM ketoprofen) to terminate the reaction. The reaction solution was then vortexed for 10 minutes and centrifuged at 4,000 rpm for 30 minutes at 4° C. Next, 400 μL of the supernatant was transferred to one new 96-well plate, centrifuged at 4,000 rpm for 30 minutes at 4° C., and 100 μL of the supernatant was transferred to a new 96-well plate ensuring the pellet was not disturbed. 100 μL of ultrapure water was added to all samples for analysis by LC-MS/MS.

The T_{1/2} was determined by the linear regression of the natural logarithm of the remaining percentage of the parent drug vs. incubation time curve. The slope value (k) of the curve was then substituted into the following equation to determine the T_{1/2}:

$$in vitro {T}_{1 / 2} = - ( \frac{0.693}{k} )$$

The in vitro intrinsic clearance (in vitro CLᵢₙₜ, in μL/min/10⁶ cells) was determined by the following equation.

$$in vitro C {L}_{i n t} = ( \frac{0.693}{{T}_{\frac{1}{2}}} ) * ( \frac{volume of incubation}{number of hepatocytes} )$$

where volume of incubation=0.2 mL and number of hepatocytes per well=0.1*10⁶ cells

The column used was a Phenomenex Synergi 4 μ Hydro-PR 80A (2.0×30 mm) with a mobile phase consisting of 0.1% formic acid in water (solvent A) and 0.1% formic acid in acetonitrile (solvent B) at room temperature. Injection volume was 10 μL. MS analysis was performed on a API 4000 instrument from AB Inc (Canada) with an ESI interface.

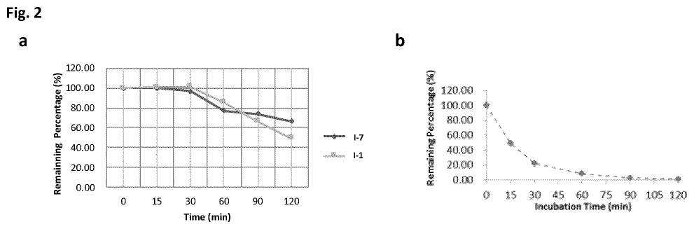

Compound I-1 was determined to have a Tv₂ of 184 minutes, while compound I-7 had a T_{1/2} of 115 minutes. (FIG. 2a) The clearance rate of Batabulin (T138067) from prior art document U.S. Pat. No. 6,482,860B1 is much faster with a T_{1/2} of 17.9 minutes. (FIG. 2b). This suggests that compounds of this application may have a slower clearance rate than the comparable Batabulin compound from literature.

### Example 72 Exemplary Compound I-1 hERG Receptor Inhibition Analysis

To assess potential toxicity with the human ether-a-go-go-related gene (hERG) receptor, I-1 was evaluated for in vitro hERG inhibition. hERG stably expressed HEK293 cells were used in this assay. Cells were induced with doxycycline at 1 μg/mL for a period of 48 hours. Induced cells are resuspended and plated on coverslips at 5×10⁵ cells/per 6 cm culture dish prior to use. Coverslips were removed from the cell culture dish and placed on microscope stage in a bath chamber. The tip of the electrode was located under the microscope, and then the electrode was advanced towards the surface of the located cell. The capacity current was removed, which was simultaneous with the voltage step, and the whole cell configuration was obtained by applying repetitive suction until the membrane patch was ruptured. The membrane potential was set to −60 mV, and the holding potential was set to −90 mV for 500 ms. The current was recorded at 50 kHz and filtered at 10 kHz. Leaking current was tested by depolarizing membrane potential to −80 mV, and the initial holding voltage was −90 mV. The hERG current was elicited at +30 mV for 4.8 seconds, and then the voltage was adjusted back to −50 mV for 5.2 seconds to remove the inactivation. The deactivating tail current was observed, of which the maximum tail current was used to determine hERG current amplitude. The current was recorded for 120 seconds. Once the hERG was maintained at stabilized baseline for 5 minutes, the working solution containing dilute concentration of compound I-1 was applied. The hERG current was recorded for 5 minutes. For dose-response study, the test compounds were tested in a cumulative manner from low to high concentrations. As a positive control, 5 doses of Dofetilide was applied.

For data analysis, the peak current inhibition (peak current was extracted from the original data by PatchMaster or Clampfit) was calculated using the equation:

Peak current inhibition=(1−peak tail current_{compound}/peak tail current blank vehicle)×100)

The results of this study (Table 8) indicate that I-1 is a weak inhibitor hERG receptor with an IC₅₀ of 17.34 μM, with FDA criterion for hERG positive drugs being IC₅₀ values<1 μM. Avoiding activity with the hERG receptor potentially reduces the chance of cardiotoxicity associated with this compound.¹⁰

TABLE 8
Inhibitory effects of I-1 and Dofetilide control on hERG
channel, evaluated via a manual patch-clamp system.

| Compound | hERG IC₅₀ (μM) |
|---|---|
| Dofetilide | 0.013 |
| I-1 | 17.34 |

### E. Exemplary Compound I-1 Low Reactivity Profile As Demonstrated by In Vitro Assays

### Example 73 Compound I-1 Activity in Tubulin Polymerization Assay

Since Batabulin is structurally comparable to a1-1 of the present application, potential inhibitory activity of I-1 against tubulin polymerization was assessed in vitro. A half-area transparent 96-well plate (Corning Cat. #3697) was pre-warmed to 37° C. for 20 minutes prior to starting the assay. Polymerization buffer (80 mM PIPES pH 6.9, 2 mM MgCl₂, 0.5 mM EGTA, 15% v/v glycerol, 1 mM GTP) was cooled to 4° C. Compounds were prepared at 100 μM in compound buffer (80 mM PIPES pH 6.9, 2 mM MgCl₂, 0.5 mM EGTA, 5% DMSO). In each well of the pre-warmed assay plate, 10 μL of compound or buffer control was added, and was then incubated at 37° C. for 3 minutes. During this time, one 200 μL vial of tubulin in general buffer (80 mM PIPES pH 6.9, 2 mM MgCl₂, 0.5 mM EGTA) was defrosted by placing in a room temperature water bath until thawed. The 200 μL of tubulin was mixed with 420 μL of cold polymerization buffer (3 mg/mL tubulin in 80 mM PIPES, pH 6.9, 2 mM MgCl₂, 0.5 mM EGTA, 1 mM GTP, 10.2% glycerol), and then 90 μL was immediately pipetted into each reaction well (final compound concentration=10 μM) and put in the Cytation 3 plate reader at 37° C. After orbital shaking for 10 seconds, absorbance was taken (340 nm) every 30 seconds for 1 hour.

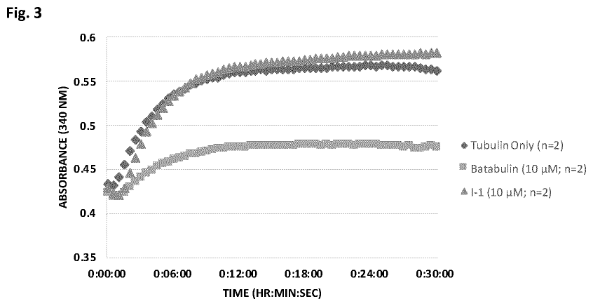

Batabulin (T138067), is a covalent inhibitor of beta-tubulin polymerization.⁸′⁹ Compound I-1 evaluated against tubulin polymerization showed no significant inhibitory action against polymerization (FIG. 3), thus indicating that I-1 may not have the same inhibitory mechanism as Batabulin.

### Example 74 Exemplary Compound I-1 Activity in a Kinase Screen (KINOMEscan DiscoverX)

Procedure from DiscoverX:

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). I-1 test compound was prepared as 40× stocks in 100% DMSO and directly diluted into the assay. All reactions were performed in polypropylene 384-well plates in a final volume of 0.02 mL. The assay plates were incubated at room temperature with shaking for 1 hour and the affinity beads were washed with wash buffer (1×PBS, 0.05% Tween 20). The beads were then re-suspended in elution buffer (lx PBS, 0.05% Tween 20, 0.5 μM non-biotinylated affinity ligand) and incubated at room temperature with shaking for 30 minutes. The kinase concentration in the eluates was measured by qPCR. To re-word

I-1 test compound was tested at 10 μM, and results for primary screen binding interactions are reported as ‘°/0 Ctrl,’ where lower numbers indicate stronger hits in the matrix. Ctrl is calculated with the following formula:

$$\% Ctrl = ( \frac{test compound signal - positive control signal}{negative control signal - positive control signal} ) \cdot 100 \%$$

test compound=I-1
negative control=DMSO (100% Ctrl)
positive control=control compound (0% Ctrl)

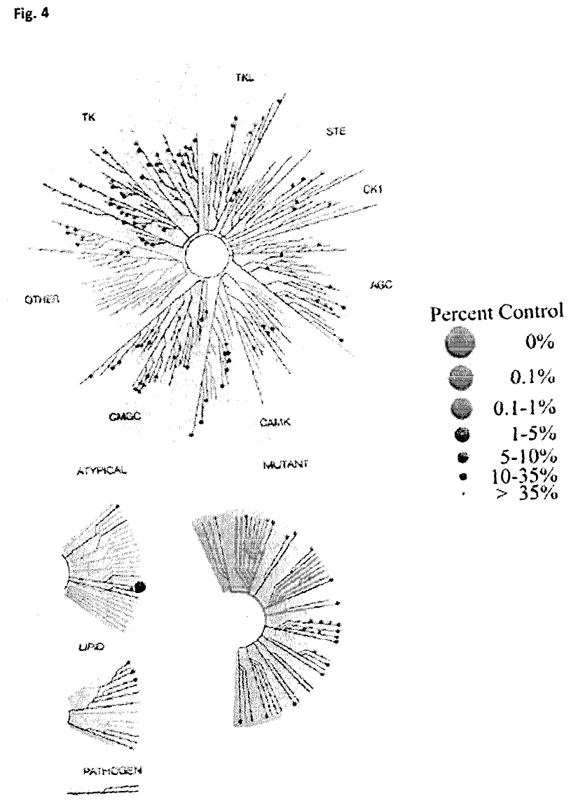

The results of this study as summarized in a TREEspot™ interaction map (FIG. 4) indicate that I-1 does not inhibit the 123 kinase targets it was screened against at 10 μM.

### Example 75 Exemplary Compound I-1 Activity in a Bromodomain Screen (BROMOscan DiscoverX)

Procedure from DiscoverX:

T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05 Tween 20, 1 mM DTT) to remove unbound ligand and to reduce non-specific phage binding. Binding reactions were assembled by combining bromodomains, liganded affinity beads, and test compounds in 1× binding buffer (16% SeaBlock, 0.32×PBS, 0.02% BSA, 0.04% Tween 20, 0.004% Sodium azide, 7.9 mM DTT). Test compounds were prepared as 1000× stocks in 100% DMSO and subsequently diluted 1:25 in monoethylene glycol (MEG). The compounds were then diluted directly into the assays such that the final concentrations of DMSO and MEG were 0.1% and 2.4%, respectively. All reactions were performed in polypropylene 384-well plates in a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

I-1 test compound was tested at 10 μM, and results for primary screen binding interactions are reported as ‘% Ctrl,’ where lower numbers indicate stronger hits in the matrix. % Ctrl is calculated with the following formula:

$$\% Ctrl = ( \frac{test compound signal - positive control signal}{negative control signal - positive control signal} ) \cdot 100 \%$$

test compound=I-1
negative control=DMSO (100% Ctrl)
positive control=control compound (0% Ctrl)

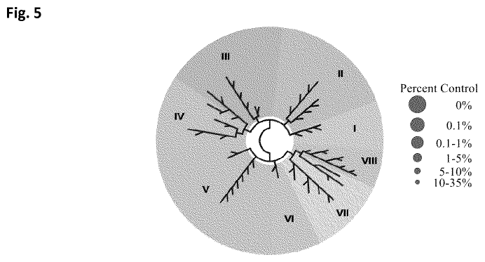

The results of this study as summarized in a TREEspot™ interaction map (FIG. 5) demonstrated that I-1 does not inhibit the 32 bromodomain targets it was screened against at 10 μM.

### Example 76 Compound I-1 ¹⁹F NMR Screen against Cys-containing Proteins

To assess potential reactivity of I-1 against Cys containing proteins, a series of ¹⁹F NMR studies were conducted with BSA, lysozyme, STAT3, and STATS. All ¹⁹F NMR experiments were recorded at 25° C. on a 600 MHz spectrometer equipped with an H(F)CN room temperature probe. All samples were prepared in 100 mM HEPES pH 7.4, and 100 μM 5-fluoro-Trp, with a final concentration of 10% D20 and 10% DMSO. The samples were incubated for 2 hours at 37° C. prior to data collection. All spectra were normalized and referenced according to the fluorine peak of 5-fluoro-Trp.

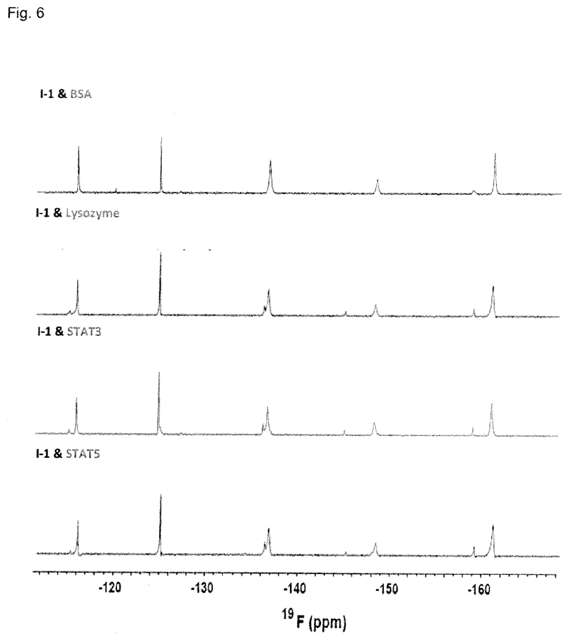

Results from this study showed I-1 to be unreactive towards Cys-containing BSA, lysozyme, and STAT3/5, with no generation of free fluoride being detected at −120 ppm to indicate covalent modification of those proteins, further supporting the stability of I-1 and selectivity for UBA5. (FIG. 6)

### F. Exemplary Compound I-1 Activity Against the UFM1 Pathway

Select exemplary compounds of this application were evaluated against UBA5 E1-activating enzyme in vitro and UFM1 cascade in cellulo. Results suggest that select compounds of this application are inhibitors of the UFMylation pathway, as evaluated in MV4-11 cells, and based on in vitro evaluation of I-1, covalently modify UBA5, likely through conjugation to UBA5's catalytic Cys250 residue. Additionally, exemplary compounds destabilize UBA5 in vitro and reduce the UBA5 levels in cellullo, which may be a result of covalent modification.

### Example 77 Protein Expression and Purification Protocols

Constructs of full length human UBA5 (1-404) were cloned into a pET28b(+) vector with an N-terminal His-SUMO tag using Ndel and Xhol restriction enzymes. Molecular cloning was performed by GenScript. Constructs were transformed in E. coli BL21 (DE3) RILP cells (Aligent). Single colonies were picked and inoculated into 5 mL of LB medium (with 50 μg/mL kanamycin and 34 μg/mL chloramphenicol). Cells were grown at 37° C. for 3-4 hours with constant shaking and then used to innculate 1 L of Super broth supplemented with 10 mM MgSO4, 0.1 glucose, 50 μg/mL kanamycin and 34 μg/mL chloramphenicol). The culture was incubated at 37° C., (275 rpm) and the optical density (0D600) was monitored. The temperature was iteratively decreased to 30, 25 and 18° C., when the OD600 reached values of 0.5, 1.0 and 1.5, respectively. After the last temperature decrease, 60 mL of 50% (v/v) ethanol was added into the growth media for a final 3% (v/v) solution. Following a 30-minute equilibration period, 0.5 mM Isopropyl β-D-1-thiogalactopyranoside (IPTG) was added to the flasks. Approximately 20 hours after induction, the cultures were harvested by centrifugation. The cell pellets were combined and stored at −80° C. before protein purification.

For protein purification, the cell pellets were re-suspended in lysis buffer in a ratio of 10 mL buffer per 1 gram (wet weight) of cell paste. The lysis buffer consisted of 20 mM NaPhos, pH 7.8, 100 mM arginine, 100 mM glutamic acid, 0.2% [v/v] Triton-X, 0.1% [v/v] Nonidet P-40 substitute [Sigma-Aldrich], 10% [v/v] glycerol, 2 mg/mL deoxycholic acid, 1 mg/mL lysozyme, 5 mM 6-aminocaproic acid, 5 mM benzamide and 1 mM phenylmethylsulfonyl fluoride. After 10 minutes of nutation, the suspension was sonicated 4×30 seconds (at an intensity setting of 20 on a Branson Sonifer-250). The cell lysates were cleared by centrifugation (14800 g for 30 minutes.) The supernatant was loaded under gravity flow onto 5 mL of Ni2+-NTA resin (GE Healthcare) that was pre-equilibrated with equilibration buffer (20 mM NaPhos, pH 7.5, 150 mM NaCl, 5 mM imidazole and 10% [v/v] glycerol). The column was washed with 10 column volumes of equilibration buffer B (20 mM NaPhos, pH 7.5, 150 mM NaCl, 25 mM imidazole, 10% [v/v] glycerol). UBA5 protein was eluted from the Ni2+-NTA column using elution buffer consisting of: 20 mM NaPhos, pH 7.2, 150 mM NaCl, 500 mM imidazole, 10% [v/v] glycerol. The eluted fraction was diluted 2-fold and Ulp1 protease was added to sample at concentration of 1:1000 to cleave the SUMO tag. The sample was concentrated using a 30 kDa cut-off filter and loaded onto a Superdex S650 gel filtration column (Bio-Rad) in equilibration buffer (20 mM NaPhos, pH 7.5, 150 mM NaCl, and 10% [v/v] glycerol) The fractions containing UBA5 were concentrated via centrifugation with 30 kDa cut-off filters and the protein concentration was determined by BCA assay. Similar procedures were conducted for untagged UFM1 and UFC1 protein expression and purifications.

### Example 82 ¹⁹F NMR-based Study of I-1 and UBA5 Enzyme Interaction In Vitro

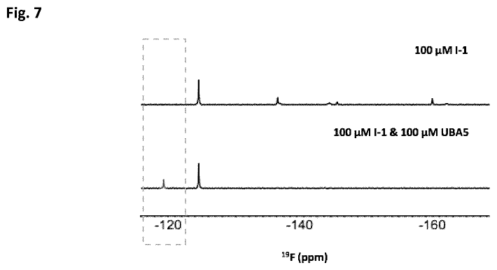

To evaluate covalent modification of UBA5 by I-1, ¹⁹F NMR studies were conducted in accordance with the procedure described in Example 80. Results of this study (FIG. 7) indicate I-1 covalently modifies UBA5 enzyme, with free fluoride detection at −120 ppm.

### Example 78 MS Based Study of I-1 and UBA5 Enzyme Interaction In Vitro

To further characterize covalent modification of UBA5 by I-1, samples were incubated as 100 μM I-1 and 50 μM UBA5 (50 mM HEPES, pH 7.4) at 30° C. for 2 hours, followed by storage overnight at 4° C. prior to submission for LC-MS analysis.

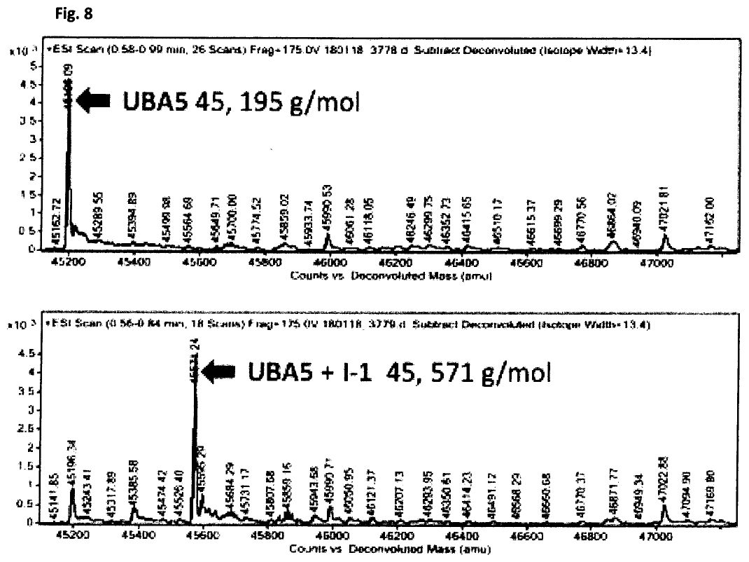

Results of MS analysis showed UBA5/I-1 covalent adduct formation at 45, 571 Da, further supporting that I-1 covalently modifies UBA5 in vitro. (FIG. 8)

### Example 79 Western Blot Analysis of UFMylation Pathway After I-1 Treatment in MV4-11 cells

Post-treatment (8 hours) of MV4-11 cells with I-1 test compound at 1, 0.5, 0.25, 0.1 and 0 μM concentrations, all cells were lysed with radioimmunoprecipitation assay (RIPA) buffer: 20 mM Tris pH 7.4, 150 mM NaCl, 0.5% deoxycholate, 1% Triton X-100, 0.1% sodium dodecyl sulfate (SDS). Total protein was measured using BCA assay (Sigma). In each assay, clarified protein were resolved on a 4%-15% polyacrylamideSDS gel and transferred to a PVDF membrane (Bio-Rad). The membranes were blocked with 5% solution of skim milk powder in TBST and incubated for at least 1 hour followed by an overnight incubation at 4° C. in primary antibody (1:1000 dilution). Blots were probed with antibodies against UBA5, UFM1, UFC1, c-Myc, and beta-actin was used as a loading control (Santa Cruz Biotechnology catalog # sc-835). The PVDF membrane then washed with TBST (3 times for 5 minutes). A horseradish peroxides (HRP)-conjugated goat anti-mouse IgG secondary antibodies (Cell signaling Catalog #7076S) was applied to the membrane (1:5000 dilution) and incubated for 1 hour at room temperature. The blots were then rinsed again 3 times in TBST for 10 minutes. Bands were visualized using clarity western ECL substrate luminal/enhancer solution and peroxide solution 1:1 ratio for HRP secondary antibody, according to the manufacturer's instructions (Bio-Rad) and analyzed using Image lab software (Bio-Rad).

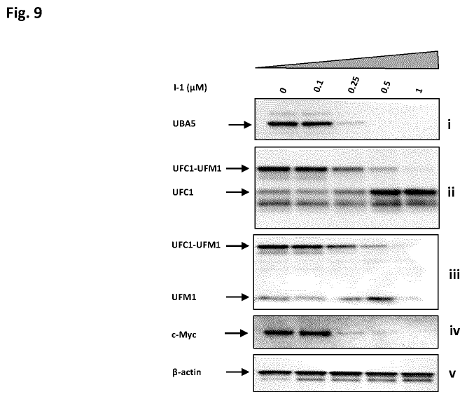

Western blot analysis of MV4-11 cells dosed with JP494 at 8 hours showed a reduction of UBA5 total protein levels at 250 nM I-1 and complete wipe-out at greater than 500 nM I-1. (FIG. 9 i) In the subsequent cellular assays, levels of UFM1-UFC1 conjugates experienced a corresponding decrease starting at 250 nM I-1. (FIG. 9 ii and iii)C-Myc levels were also decreased starting at 250 nM I-1. (FIG. 9 iv) These results would suggest that I-1 is an inhibitor of UFMylation pathway in MV4-11 cells.

### Example 80 Additional Compounds Activity against UFMylation Pathway in MV4-11

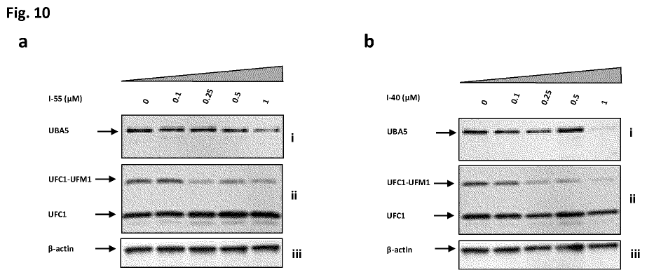

Following the procedure outlined in Example 69, select compounds were screened in MV4-11 cells for UFM1 pathway inhibition. Under the current conditions, compounds I-55 and I-40 (FIG. 10 a & b) had comparable activity to I-1, resulting in reductions of UBA5 levels as well as UFC1-UFM1 conjugate levels.

### Example 81 In vitro Transthiolation Assays of Exemplary Compounds

To further evaluate UBA5 inhibition of exemplary compounds, in vitro transthiolation assays were conducted, wherein levels of UFC1-UFM1 conjugate formation was monitored with compound concentrations of 10 and 50 μM. UBA5 and test compounds were pre-incubated for a period of 8 hours at 37° C. prior to addition of other assay components. After this period, UFM1, UFC1 and ATP were introduced to initiate the reactions. Final concentrations in the reactions were 250 nM UBA5, 10 μM UFM1, 10 μM UFC1 and 100 μM ATP for total assay volumes of 20 μL, in buffer consisting of 50 mM 4-(2-hydroxyethyl)-1-piperazineethanesulfonic acid (HEPES),), 0.5 mM TCEP, 5 mM MgCl₂, at pH 7.4. A final concentration of 10% (v/v) DMSO was present in all reactions. The reactions were allowed to proceed for 1 hour before stopping with 4× Laemmli buffer (Bio-Rad 1610747). Samples were then run on 4-20% non-reducing SDS-PAGE stain-free gels to separate protein conjugates. Gels were subsequently imaged using Stain-free imaging technology (Bio-Rad). Image Lab software was used to quantify the intensity of the UFM1-UFC1 conjugate bands to controls with no inhibitor present and reported as % inhibition values.

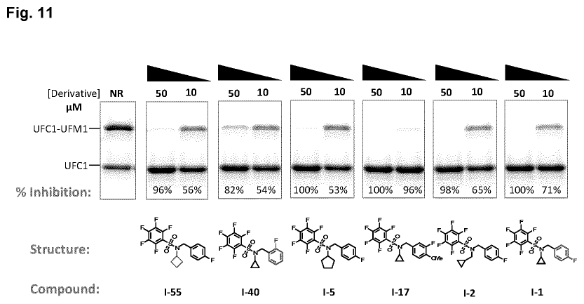

Select exemplary compounds were tested in this transthiolation assay, with variable inhibitory activity being observed at 10 and 50 μM against UFM1-UFC1 conjugate formation. (FIG. 11)

I-1 was evaluated as well using differing transthiolation assay conditions, with a reduction in UBA5 concentration to 50 nM and a pre-incubation period of 3.5 hours at 37° C. of UBA5 with I-1. This was followed by a 30 minute reaction time after UFM1, UFC1 and ATP addition. A final concentration of 5% (v/v) DMSO was used.

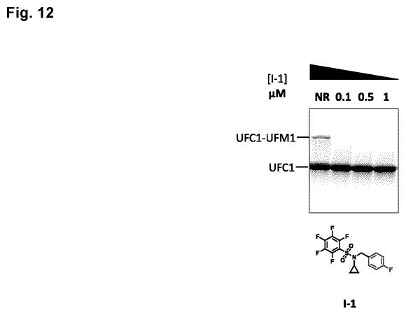

Using these new conditions I-1 potency was improved to no conjugate formation at sub-micromolar concentrations. (FIG. 12)

### Example 82 Thermal Shift Assay Analysis of UBA5 and Exemplary Compound I-1

SYPRO Orange protein gel stain (Sigma-Aldrich) was used to conduct thermal shift assays. This was done on a Bio-Rad C1000 Touch ThermoCycler with a CFX96 Real-Time optical unit. Final UBA5 protein concentrations of 0.5 μM were used in 50 mM HEPES, pH 7.4. UBA5 was pre-incubated with test compound I-1 for 4 hours at 30° C. Next, SYPRO orange was added to the samples for a final 5× (10 μM) concentration from the original 5000× stock. DMSO content was 5% (v/v). Heating was done in 0.5° C. increases with 30 seconds in between, from 10 to 75° C. Fluorescence intensity was measured at 560-580 nM followed by excitation at 450-490 nM. This emission intensity was graphed against temperature and then recorded as a first derivative curve. The temperature of the resultant curve minima provided the melting temperature (Tₘ) of the protein. Experiments were run in triplicates.

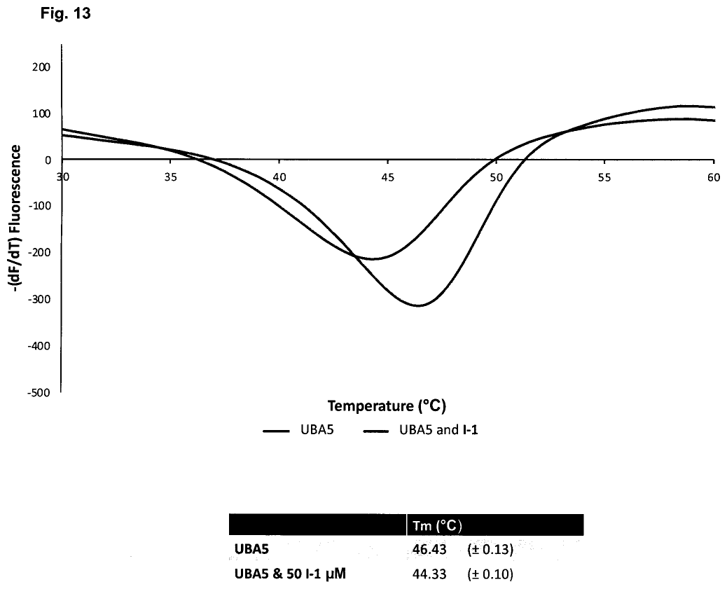

Results from this experiment indicated that I-1 incubation with UBA5 results in protein destabilization, with a Tₘ shift from 46.43° C. to 44.33° C. This may explain why in MV4-11 cells total UBA5 levels decrease, potentially through a destabilization then subsequent degradation mechanism. (FIG. 13)

### Example 82 Density Functional Theory (DFT) Calculations

Calculations were performed using Gaussian 16 at the wB97X-D level of theory, using the 6-31++G** basis set for all atoms, with IEFPCM water solvent correction. Ground states were confirmed by vibrational analysis to have zero imaginary frequencies and all transition states (TS1) were confirmed to have a single imaginary frequency of approximately −290 cm⁻¹.

Table 9 presents results of DFT calculations for select exemplary compounds.

TABLE 9
DFT calculations for select exemplary compounds, and as a comparison to reactivity, most
reactive compounds listed on top and least reactive towards the bottom.

| | | ΔG^{‡} TS1 | |
|---|---|---|---|
| Structures | I-Code | (kcal/mol) | |
| 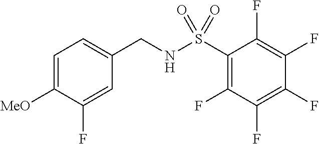 [SMILES: COc1ccc(CNS(=O)(=O)c2c(F)c(F)c(F)c(F)c2F)cc1F] | n/a | 15.7 | Most Reactive |
| 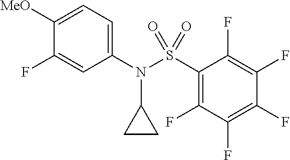 [SMILES: COc1ccc(N(C2CC2)S(=O)(=O)c2c(F)c(F)c(F)c(F)c2F)cc1F] | n/a | 15.50 | |
| 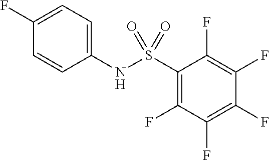 [SMILES: O=S(=O)(Nc1ccc(F)cc1)c1c(F)c(F)c(F)c(F)c1F] | Comparative Example 5 | 15.71 | |
| 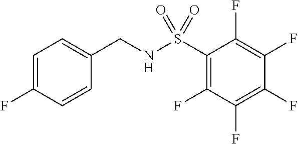 [SMILES: O=S(=O)(NCc1ccc(F)cc1)c1c(F)c(F)c(F)c(F)c1F] | Comparative Example 4 | 15.75 | |
| 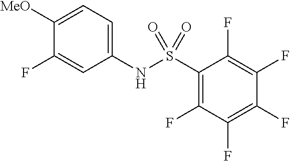 [SMILES: COc1ccc(NS(=O)(=O)c2c(F)c(F)c(F)c(F)c2F)cc1F] | T138067 | 15.87 | |
| 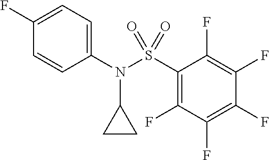 [SMILES: O=S(=O)(c1c(F)c(F)c(F)c(F)c1F)N(c1ccc(F)cc1)C1CC1] | n/a | 16.09 | |
| 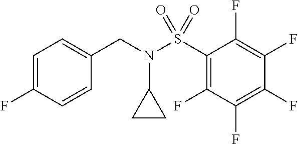 [SMILES: O=S(=O)(c1c(F)c(F)c(F)c(F)c1F)N(Cc1ccc(F)cc1)C1CC1] | I-1 | 16.37 | |
| 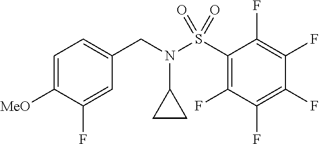 [SMILES: COc1ccc(CN(C2CC2)S(=O)(=O)c2c(F)c(F)c(F)c(F)c2F)cc1F] | I-17 | 16.41 | Least Reactive |

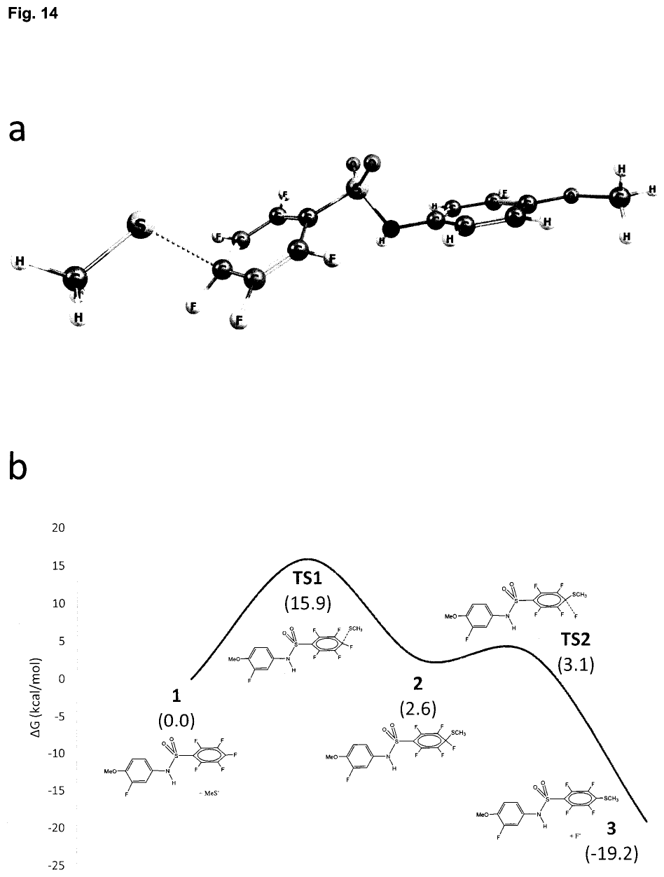

Overall reaction rate is limited by the free energy of the first transition state (ΔG^{‡} TS1 (kcal/mol)) and the transition state of fluoride dissociation is nearly barrierless. Therefore the 0.5 kcal/mol increase in free energy for I-1 compared to Batabulin (T138067) calculated by DFT may explain the increase in stability observed towards thiol/thiolate nucleophiles. (FIG. 14) This data supports that both R¹ substituents and a benzyl as opposed to phenyl groups are needed to yield improved metabolic stability.

### Example 83 Patient Derived GBM BTIC Cell Lines Screens of Exemplary Compounds

Glioblastoma (GBM) tumor cells with stem-cell properties termed brain tumor initiating cells (BTICs,) are enriched using serum-free culture. Effects of select exemplary compounds were tested on cell proliferation in patient derived GBM BTIC lines: GBM8 and BT428. GBM BTICs were cultured in NeuroCult™ NS-A Proliferation Medium (STEMcell Technologies) supplemented with epidermal growth factor (20 ng/mL), basic fibroblast growth factor (10 ng/mL) and 2 ug/mL of Heparin. The cells were dissociated into single cells and viable cells sorted into 96 well plate at a density of 1000 cells/well. The cells were then treated with varying doses of selected compounds (250 nM, 125 nM, 62.5 nM) with three technical replicates per dilution. DMSO was used as control. Four days following the addition of compounds, the proliferative capacity of GBM BTICs was assessed using PrestoBlue Cell Viability reagent (Invitrogen).

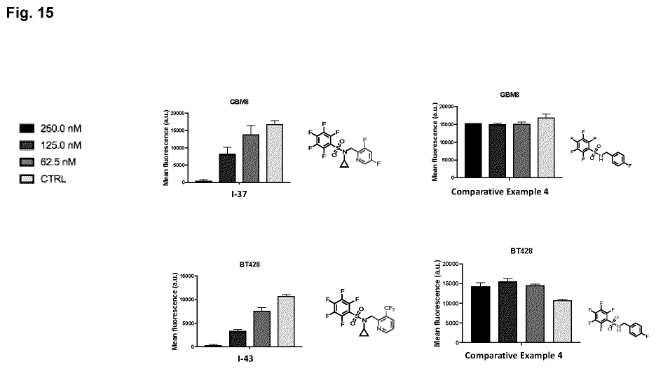

These results indicated variable activity of exemplary compounds against patient derived GBM BTIC lines. In particular, I-37 and I-43 show potency in GBM8 and BT428 respectively. (FIG. 15) Activity in other GBM samples, with lower amount of CD133 was less pronounced. Since CD133+ cells are linked to c-Myc dependency, this may further support that the present compounds may be effective for cancers which are generally dependent on c-Myc.¹¹

### FULL CITATION FOR DOCUMENTS REFERRED TO IN THE SPECIFICATION

1. Schulman, B. A. & Wade Harper, J. Ubiquitin-like protein activation by E1 enzymes: The apex for downstream signalling pathways. Nature Reviews Molecular Cell Biology 10, 319-331 (2009).
2. Tatsumi, K. et al. A Novel Type of E3 Ligase for the Ufml Conjugation System. J. Biol. Chem. 285, 5417-5427 (2010).
3. Yoo, H. M. et al. Modification of ASC1 by UFM1 is crucial for ERa transactivation and breast cancer development. Mol. Cell 56, 261-74 (2014).
4. Lemaire, K. et al. Ubiquitin fold modifier 1 (UFM1) and its target UFBP1 protect pancreatic beta cells from ER stress-induced apoptosis. PLoS One 6, (2011).
5. Tatsumi, K. et al. The Ufml-activating enzyme Uba5 is indispensable for erythroid differentiation in mice. Nat. Commun. 2, 181 (2011).
6. Meyers, R. M. et al. Computational correction of copy number effect improves specificity of CRISPR-Cas9 essentiality screens in cancer cells. Nat. Genet. 49, 1779-1784 (2017).
7. McFarland, J. M. et al. Improved estimation of cancer dependencies from large-scale RNAi screens using model-based normalization and data integration. bioRxiv 305656 (2018). doi:10.1101/305656
8. Flygare, J. A., Medina, J. C., Shan, B., Clark, D. L., Rosen, T. J. Pentafluorobenzenesulfonamide and analogs. (2002).
9. Shan, B. et al. Selective, covalent modification of beta-tubulin residue Cys-239 by T138067, an antitumor agent with in vivo efficacy against multidrug-resistant tumors. Proc. Natl. Acad. Sci. U.S.A 96, 5686-91 (1999).
10. Recanatini, M., Poluzzi, E., Masetti, M., Cavalli, A. & De Ponti, F. QT prolongation through hERG K+ channel blockade: Current knowledge and strategies for the early prediction during drug development. Med. Res. Rev. 25, 133-166 (2005).
11. Wang, J. et al. c-Myc is required for maintenance of glioma cancer stem cells. PLoS One 3, e3769 (2008).

1. A compound of Formula I or a pharmaceutically acceptable salt and/or solvate thereof: 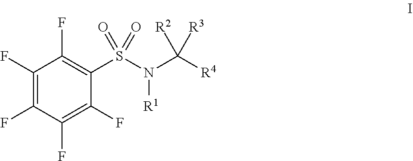
wherein:
R¹ is selected from C₁₋₁₀alkyl, C₂₋₁₀alkenyl, C₂₋₁₀alkynyl, C(O)C₁₋₁₀alkyl, C₃₋₁₀cycloalkyl, aryl, heterocycloalkyl, heteroaryl, CH₂C₃₋₁₀cycloalkyl, CH₂aryl, CH₂heterocycloalkyl and CH₂heteroaryl, the latter 8 of which are each optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆ alkyl, C(O)C₂₋₆ alkenyl, C(O)C₂₋₆ alkynyl, C(O)OC₁₋₆ alkyl, C(O)OC₂₋₆ alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl) and NHC(O)C₁₋₆alkyl;
R², and R³ are each independently selected from H, C₁₋₆alkyl, C₂₋₆alkenyl and C₂₋₆alkynyl; or
both R² and R³ combine to form ═O, or
R² and R³ together with the carbon to which they are attached form C₃₋₆ cycloalkyl;
R⁴ is selected from aryl, heteroaryl, heterocycloalkyl, C₃₋₁₀cycloalkyl, C≡C-aryl, C≡C-heteroaryl, and C≡C-heterocycloalkyl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆ alkyl), N(C₁₋₆ alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆ alkenyl, C(O)OC₂₋₆ alkynyl, S(O)ₓC₁₋₆ alkyl, S(O)ₓC₂₋₆ alkenyl, S(O)ₓC₂₋₆ alkynyl, C(O)NH₂, C(O)NHC₁₋₆alkyl, C(O)N(C₁₋₆alkyl(C₁₋₆alkyl), NHC(O)C₁₋₆alkyl and R⁵;
R⁵ is selected from Z—C₃₋₁₀cycloalkyl, Z-heterocycloalkyl, Z-aryl and Z-heteroaryl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆ alkynyl, C(O)OC₁₋₆ alkyl, C(O)OC₂₋₆ alkenyl, C(O)OC₂₋₆ alkynyl, S(O)ₓC₁₋₆ alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl), NHC(O)C₁₋₆ alkyl, C₃₋₁₀cycloalkyl, aryl, heteroaryl and heterocycloalkyl, the latter four groups being further optionally substituted by C₁₋₆alkyl, C(O)C₁₋₆alkyl and benzyl;
x is 0, 1 or 2;
Z is selected from a direct bond, C₁₋₄ alkylene, O, NH, S, SO and SO₂ and
all alkyl, alkenyl, alkynyl, aryl, heteroaryl, heterocycloalkyl and alkylene groups are optionally halosubstituted, provided that when R¹ is CH₂C₃₋₁₀cycloalkyl, the cycloalkyl group is not substituted with C(O)OC₁₋₆ alkyl and when R¹ is cyclopropyl, R⁴ is not phenyl substituted with quinazoline.

2. The compound of claim 1, wherein R¹ is selected from C₃₋₁₀cycloalkyl and heterocycloalkyl, each of which is optionally substituted with one to two of halo, CN, OH, NH₂, ═O, C₁₋₆ alkyl, C₂₋₆alkenyl, C₂₋₆alkenyl, NH(C₁₋₄alkyl), N(C₁₋₄alkyl)(C₁₋₄alkyl), OC₁₋₄alkyl, OC₂₋₄alkenyl, OC₂₋₄alkynyl, C₁₋₄alkyleneOC₁₋₄alkyl, C₁₋₄alkyleneOC₂₋₄alkenyl, C₁₋₄alkyleneOC₂₋₄alkynyl, C(O)C₁₋₄alkyl, C(O)C₂₋₄alkenyl, C(O)C₂₋₄alkynyl, C(O)OC₁₋₄ alkyl, C(O)OC₂₋₄ alkenyl, C(O)OC₂₋₄alkynyl, S(O)ₓC₁₋₄ alkyl, S(O)ₓC₂₋₄alkenyl, S(O)ₓC₂₋₄alkynyl C(O)NHC₁₋₄alkyl, C(O)N(C₁₋₄alkyl(C₁₋₄alkyl) and NHC(O)C₁₋₄alkyl).

3. The compound of claim 1, wherein R¹ is a C₃₋₆cycloalkyl optionally substituted with one or two substituents independently selected from OC₁₋₄ alkyl, C₁₋₄ alkyleneOC₁₋₄ alkyl, C₁₋₄ alkyleneOC₂₋₄alkynyl, C(O)C₁₋₄alkyl, C(O)C₂₋₄alkynyl, C(O)OC₁₋₄alkyl, and C(O)OC₂₋₄alkynyl.

4. The compound of claim 3, wherein R¹ is unsubstituted cyclopropyl, cyclobutyl, cyclopentyl or cyclohexyl.

5. The compound of claim 3, wherein R¹ is unsubstituted cyclopropyl or cyclopropyl substituted with one substituent selected from C₁₋₂alkyleneOC₁₋₄alkyl, C₁₋₂alkyleneOC₂₋₄alkynyl, C(O)C₁₋₄alkyl, C(O)C₂₋₄alkynyl, C(O)OC₁₋₄alkyl, and C(O)OC₂₋₄alkynyl.

6. The compound of claim 1, wherein R¹ is selected from heterocycloalkyl, aryl and heteroaryl, each of which is optionally substituted with one or more of C₁₋₄ alkyl, C₂₋₄ alkenyl, C₂₋₄alkynyl halo, OH, ═O, NH₂, NH(C₁₋₄alkyl), N(C₁₋₄alkyl)(C₁₋₄alkyl), OC₁₋₆alkyl, OC₂₋₆alkenyl, OC₂₋₆alkynyl, C₁₋₆ alkyleneOC₁₋₆ alkyl, C₁₋₆ alkyleneOC₂₋₆ alkenyl, C₁₋₆ alkyleneOC₂₋₆ alkynyl, C(O)C₁₋₆alkyl, C(O)C₂₋₆ alkenyl, C(O)C₁₋₆ alkynyl, C(O)OC₁₋₆ alkyl, C(O)OC₂₋₆ alkenyl, C(O)OC₂₋₆ alkynyl, C(O)NHC₁₋₆alkyl and C(O)N(C₁₋₆alkyl(C₁₋₆alkyl).

7. The compound of claim 6, wherein R¹ is selected from furanyl, indolinyl, 1,2,3,4-tetrahydroquinolinyl and 1,2,3,4-tetrahydroisoquinolinyl attached through the nitrogen in R¹.

8. The compound of claim 1, wherein R¹ is unsubstituted oxetane or tetrahydrofuran, or oxetane or tetrahydrofuran substituted with one or more of two of halo, CN, OH, NH₂, ═O, C₁₋₆alkyl, C₂₋₆alkenyl, C₂₋₆alkenyl, NH(C₁₋₄alkyl), N(C₁₋₄alkyl)(C₁₋₄alkyl), OC₁₋₄alkyl, OC₂₋₄alkenyl, OC₂₋₄alkynyl, C₁₋₄ alkyleneOC₁₋₄ alkyl, C₁₋₄ alkyleneOC₂₋₄ alkenyl, C₁₋₄ alkyleneOC₂₋₄ alkynyl, C(O)C₁₋₄alkyl, C(O)C₂₋₄alkenyl, C(O)C₂₋₄alkynyl, C(O)OC₁₋₄alkyl, C(O)OC₂₋₄alkenyl, C(O)OC₂₋₄alkynyl, S(O)ₓC₁₋₄alkyl, S(O)ₓC₂₋₄alkenyl, S(O)ₓC₂₋₄alkynyl C(O)NHC₁₋₄alkyl, C(O)N(C₁₋₄alkyl(C₁₋₄alkyl) and NHC(O)C₁₋₄alkyl).

9. The compound of claim 1, wherein R¹ is CH₂C₃₋₁₀cycloalkyl optionally substituted with one to two of halo, CN, OH, NH₂, ═O, CO₂H, SO₂F, C₁₋₆alkyl, C₂₋₆alkenyl, C₂₋₆alkenyl, NH(C₁₋₄alkyl), N(C₁₋₄alkyl)(C₁₋₄alkyl), OC₁₋₄alkyl, OC₂₋₄alkenyl, OC₂₋₄alkynyl, C₁₋₄alkyleneOC₁₋₄alkyl, C₁₋₄alkyleneOC₂₋₄alkenyl, C₁₋₄alkyleneOC₂₋₄alkynyl, C(O)C₁₋₄alkyl, C(O)C₂₋₄alkenyl, C(O)C₂₋₄alkynyl, C(O)OC₁₋₄alkyl, C(O)OC₂₋₄alkenyl, C(O)OC₂₋₄alkynyl, S(O)ₓC₁₋₄alkyl, S(O)ₓC₂₋₄alkenyl, S(O)ₓC₂₋₄alkynyl C(O)NHC₁₋₄alkyl, C(O)N(C₁₋₄alkyl(C₁₋₄alkyl) and NHC(O)C₁₋₄alkyl).

10. The compound of claim 9, wherein R¹ is unsubstituted CH₂cyclopropyl, CH₂cyclobutyl, CH₂cyclopentyl or CH₂cyclohexyl.

11. The compound of claim 9, wherein R¹ is unsubstituted CH₂cyclopropyl or CH₂cyclopropyl substituted with one substituent selected from C₁₋₂alkyleneOC₁₋₄alkyl, C₁₋₂alkyleneOC₂₋₄alkynyl, C(O)C₁₋₄alkyl, C(O)C₂₋₄alkynyl, C(O)OC₁₋₄alkyl, and C(O)OC₂₋₄alkynyl.

12. The compound of claim 1, wherein R¹ is CH₂heteroaryl, optionally substituted with one to two of halo, CN, OH, NH₂, ═O, CO₂H, SO₂F, C₁₋₆alkyl, C₂₋₆alkenyl, C₂₋₆alkenyl, NH(C₁₋₄alkyl), N(C₁₋₄alkyl)(C₁₋₄alkyl), OC₁₋₄alkyl, OC₂₋₄alkenyl, OC₂₋₄alkynyl, C₁₋₄alkyleneOC₁₋₄alkyl, C₁₋₄alkyleneOC₂₋₄alkenyl, C₁₋₄ alkyleneOC₂₋₄ alkynyl, C(O)C₁₋₄ alkyl, C(O)C₂₋₄ alkenyl, C(O)C₂₋₄ alkynyl, C(O)OC₁₋₄alkyl, C(O)OC₂₋₄alkenyl, C(O)OC₂₋₄alkynyl, S(O)ₓC₁₋₄alkyl, S(O)ₓC₂₋₄alkenyl, S(O)ₓC₂₋₄alkynyl C(O)NHC₁₋₄ alkyl, C(O)N(C₁₋₄ alkyl(C₁₋₄ alkyl) and NHC(O)C₁₋₄ alkyl).

13. The compound of claim 12, wherein R¹ is unsubstituted CH₂pyridine, CH₂pyrazine, CH₂pyrimidine, CH₂pyridazine, CH₂thiophene, CH₂furan, CH₂pyrrole, CH₂imidazole, CH₂thiazole, CH₂oxazole, CH₂pyrazole, CH₂isothiazole or CH₂isoxazole.

14. The compound of claim 13, wherein R¹ is unsubstituted CH₂pyridine.

15. The compound of claim 1, wherein R¹ is selected from C₁₋₁₀alkyl, C₂₋₁₀alkenyl, and C₂₋₁₀alkynyl.

16. The compound of claim 1, wherein R¹ is unsubstituted C₁₋₁₀alkyl.

17. The compound of claim 16 wherein, R¹ is methyl or ethyl.

18. The compound of claim 1, wherein R¹ is selected from: 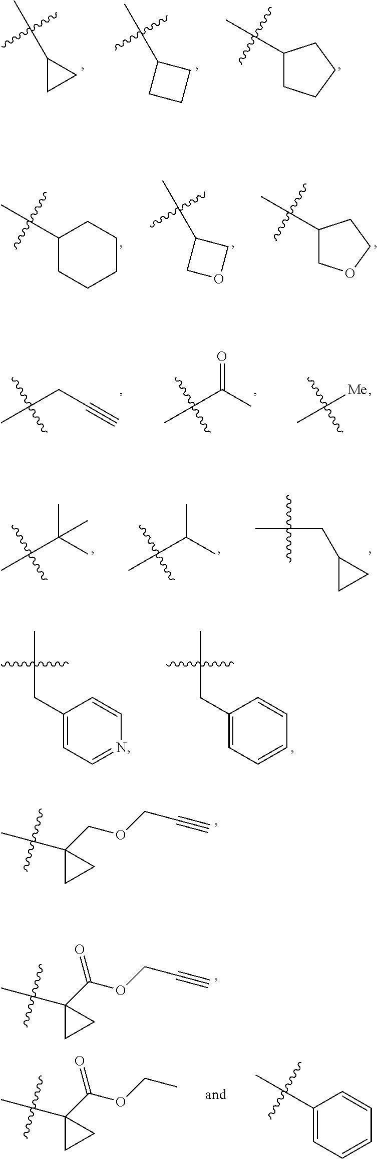
wherein the wavy line represents the point of attachment to the rest of the structure of Formula I.

19. The compound of any one of claims 1 to 18, wherein R² and R³ are each independently selected from H, C₁₋₆alkyl, C₁₋₆fluoroalkyl and C₃₋₁₀cycloalkyl.

20. The compound of claim 19, wherein, at least one of R² and R³ is H.

21. The compound of claim 20, wherein both R² and R³ are H.

22. The compound of any one of claims 1 to 18, wherein both R² and R³ combine to form ═O.

23. The compound of any one of claims 1 to 18 wherein both R² and R³ together with the carbon to which they are attached form C₃₋₆cycloalkyl.

24. The compound of claim 23, wherein both R² and R³ together with the carbon to which they are attached form cyclopentyl

25. The compound of any one of claims 1 to 24, wherein R⁴ selected from phenyl, pyridinyl, quinazolinyl, quinolinyl, indanyl, pyrazolyl, isooxazole, quinazoline and pyrrolo[2,3-b]pyridinyl optionally substituted with one, two or three F, Br, Cl, CF₃, CF₃O, CO₂H, CN, CONH₂, CO₂C₁₋₆alkyl C₃₋₆cycloalkyl, C₃₋₆heterocycloalkyl, C₁₋₄alkyl, OC₁₋₄alkyl, C₁₋₄alkynyl, OC₁₋₄alkynyl, NH₂, NHC₁₋₄alkyl N(C₁₋₄alkyl)₂, NHC(O)C₁₋₄alkyl, SO₂C₁₋₄alkyl, phenyl and heteroaryl, wherein the phenyl, heteroaryl, cycloalkyl and heterocycloalkyl groups are independently further optionally substituted with one, two or three F, Br, Cl, CF₃, CF₃O, CO₂H, CN, CONH₂, CO₂C₁₋₆alkyl C₃₋₆cycloalkyl, C₃₋₆heterocycloalkyl, C₁₋₄alkyl, OC₁₋₄alkyl, C₁₋₄alkynyl, OC₁₋₄alkynyl, NH₂, NHC₁₋₄alkyl N(C₁₋₄alkyl)₂, NHC(O)C₁₋₄ alkyl, and SO₂C₁₋₄ alkyl.

26. The compound of any one of claims 1 to 24 wherein R⁴ is selected from C≡C-aryl, C≡C-heteroaryl, and C≡C-heterocycloalkyl, each of which is optionally substituted with one or more of halo, CN, OH, NH, ═O, CO₂H, SO₂F, C₁₋₁₀alkyl, C₂₋₁₀alkenyl, C₂₋₁₀alkynyl, NH(C₁₋₆ alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆ alkenyl, C(O)OC₂₋₆ alkynyl, S(O)ₓC₁₋₆ alkyl, S(O)ₓC₂₋₆ alkenyl, S(O)ₓC₂₋₆ alkynyl C(O)NHC₁₋₆alkyl, C(O)N(C₁₋₆alkyl(C₁₋₆alkyl), and NHC(O)C₁₋₆alkyl.

27. The compound of claim 26 wherein R⁴ is selected from C≡C-aryl wherein aryl is unsubstituted phenyl or phenyl substituted with one, two or three F, Br, Cl, CF₃, CF₃O, CO₂H, CN, CONH₂, CO₂C₁₋₆alkyl, C₃₋₆cycloalkyl, C₃₋₆heterocycloalkyl, C₁₋₄alkyl, OC₁₋₄alkyl, C₁₋₄alkynyl, OC₁₋₄alkynyl, NH₂, NHC₁₋₄alkyl N(C₁₋₄alkyl)₂, NHC(O)C₁₋₄alkyl, and SO₂C₁₋₄alkyl.

28. The compound of any one of claims 1 to 27, wherein R⁴ is selected from: 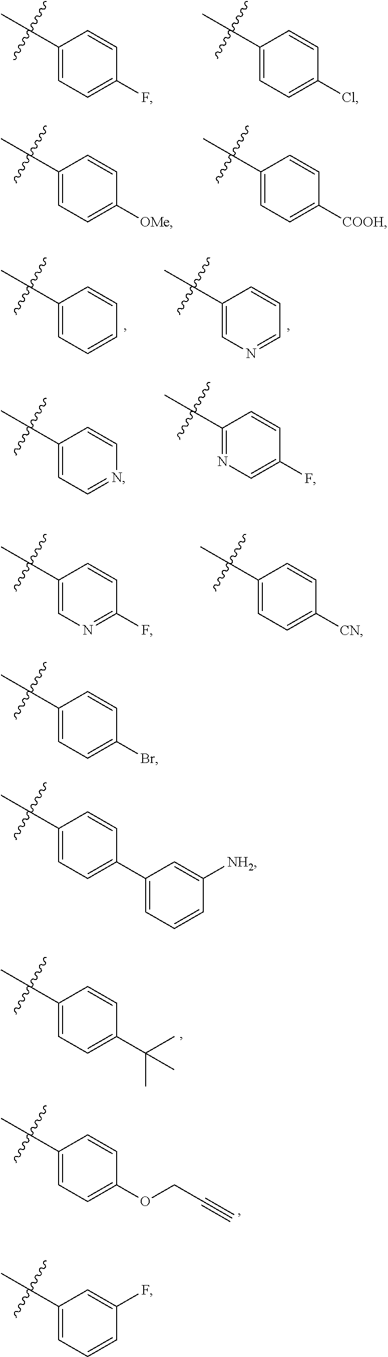 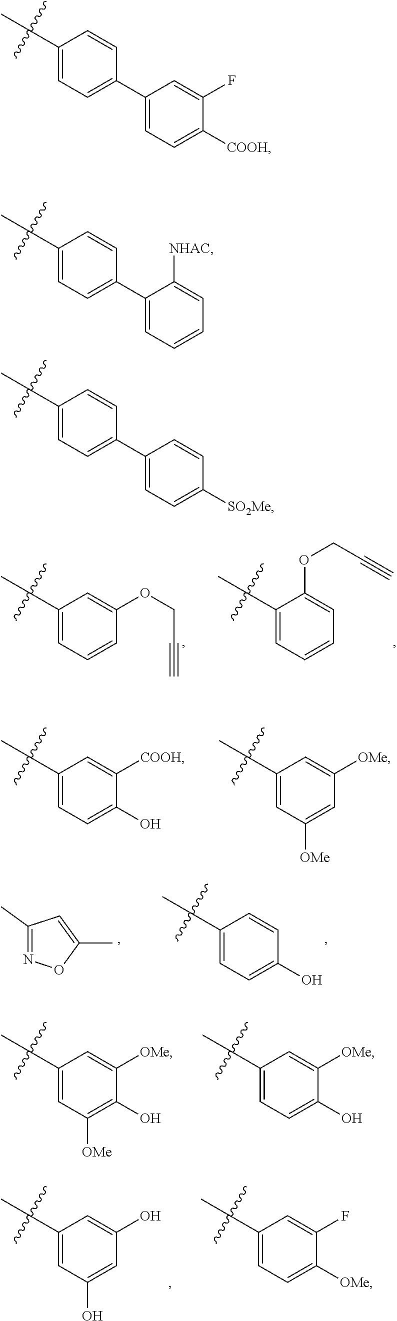 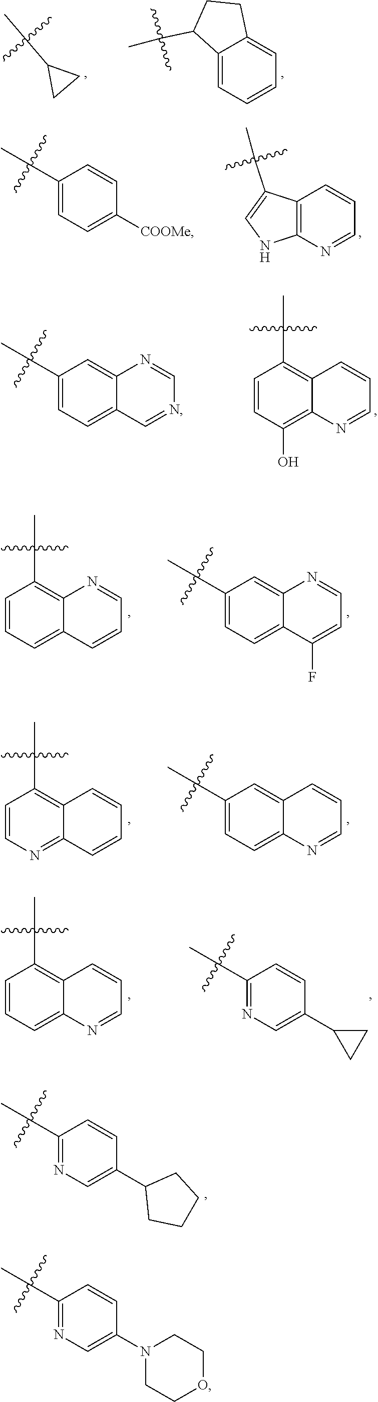 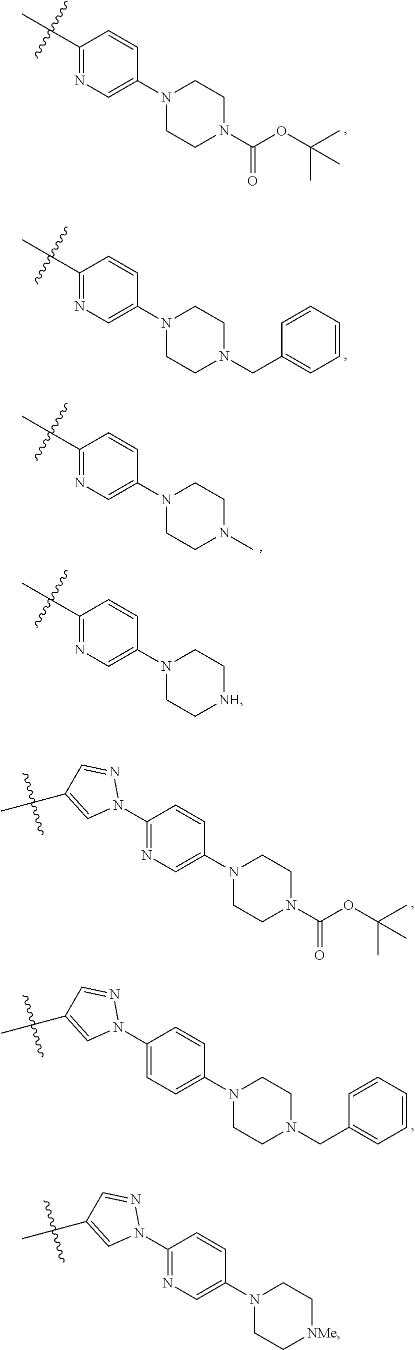 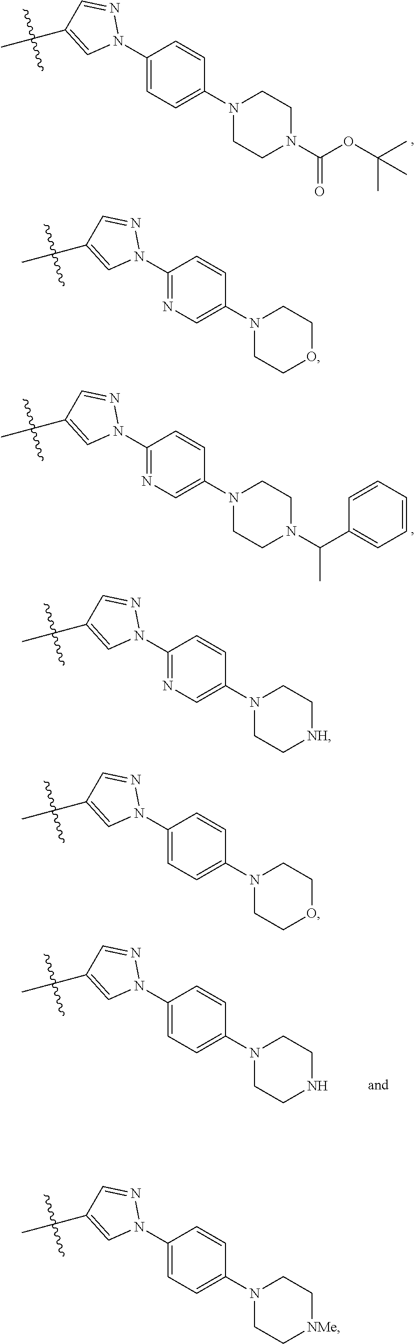
where the wavy line represents the point of attachment to the rest of the structure of Formula I.

29. The compound of any one of claims 1 to 27, wherein R⁴ is selected from: 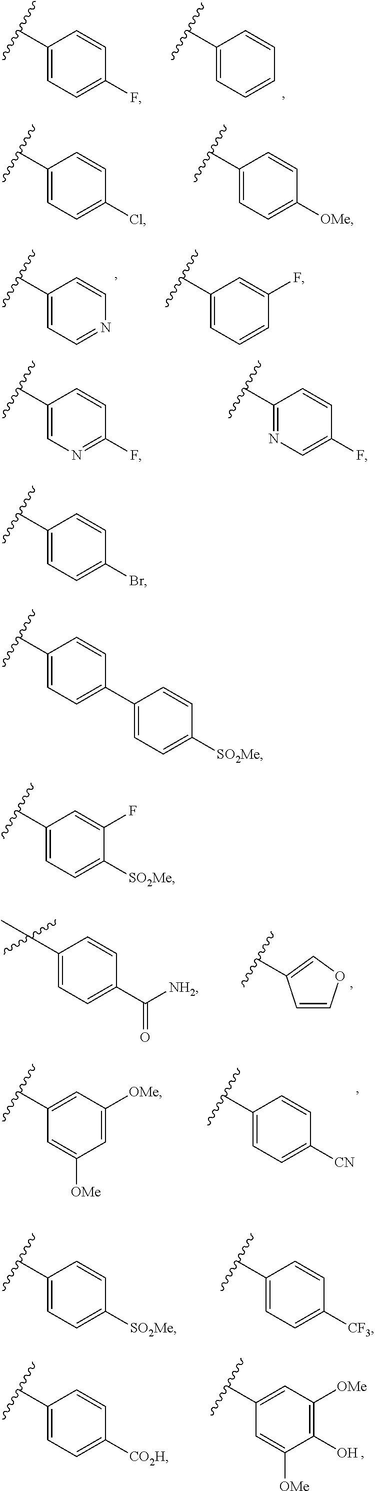 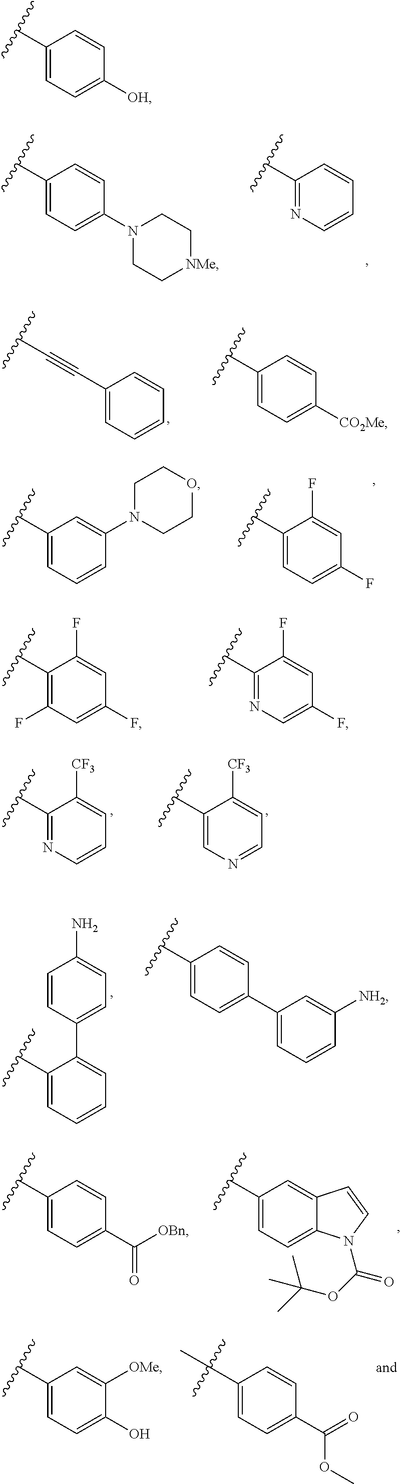 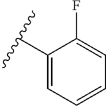 [SMILES: *c1ccccc1F]
where the wavy line represents the point of attachment to the rest of the structure of Formula I.

30. The compound of claim 1 selected from:

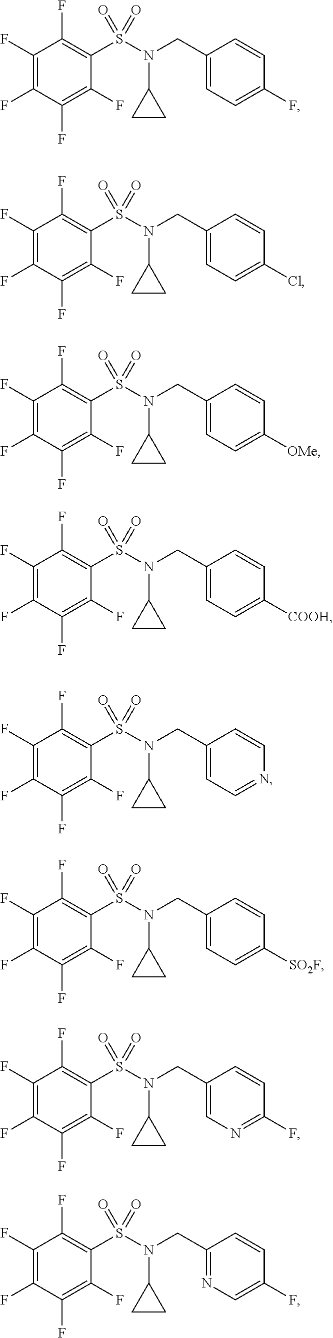

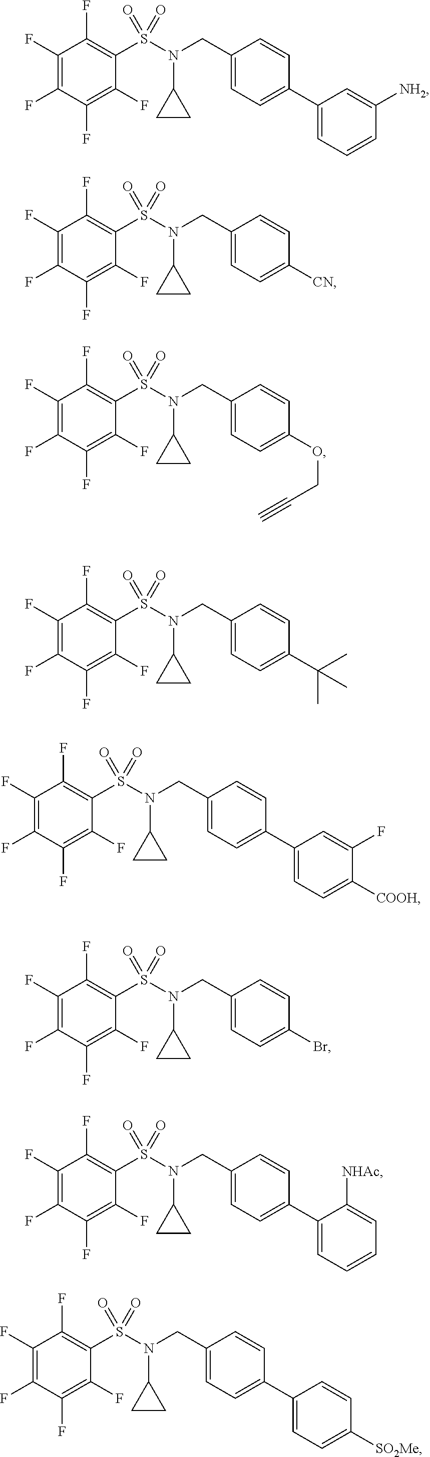

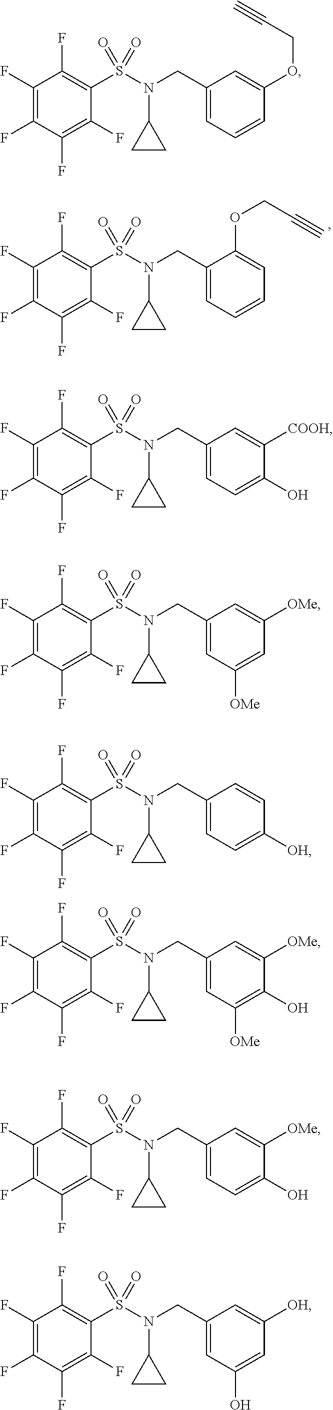

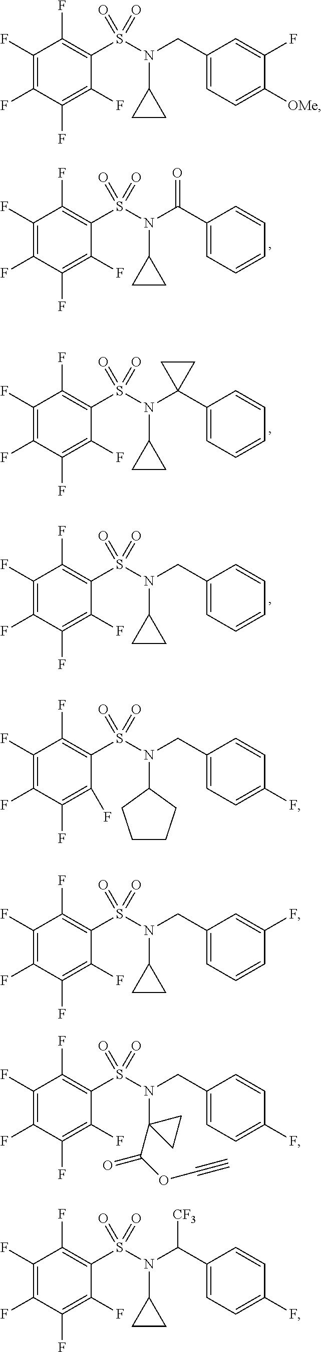

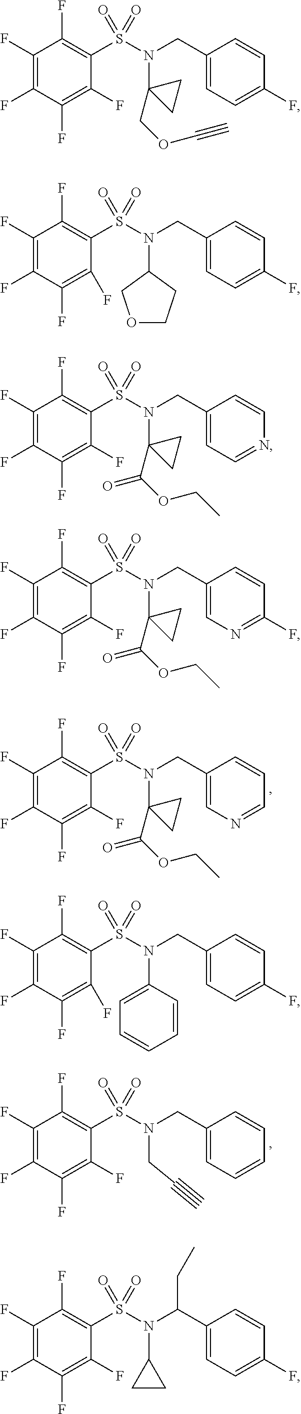

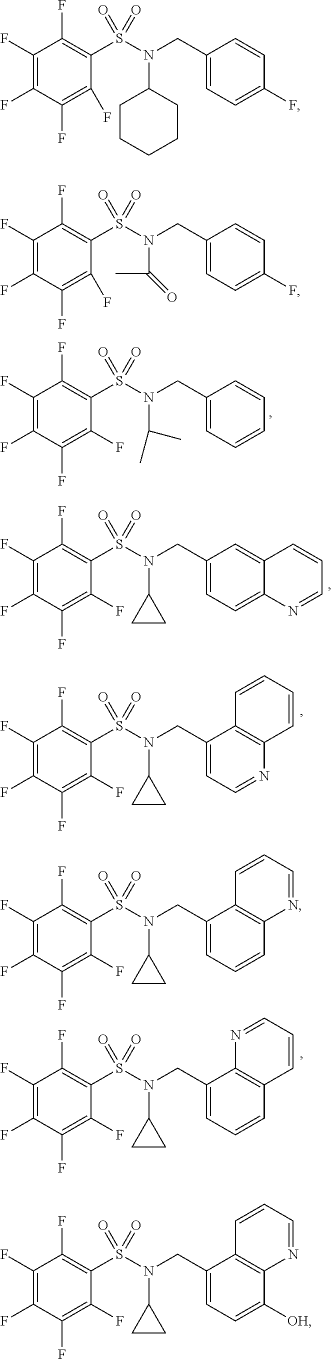

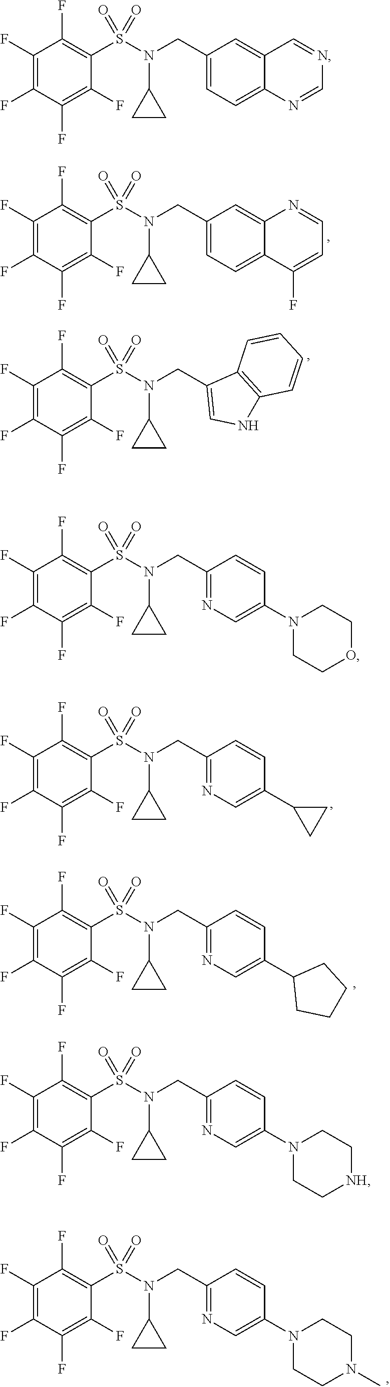

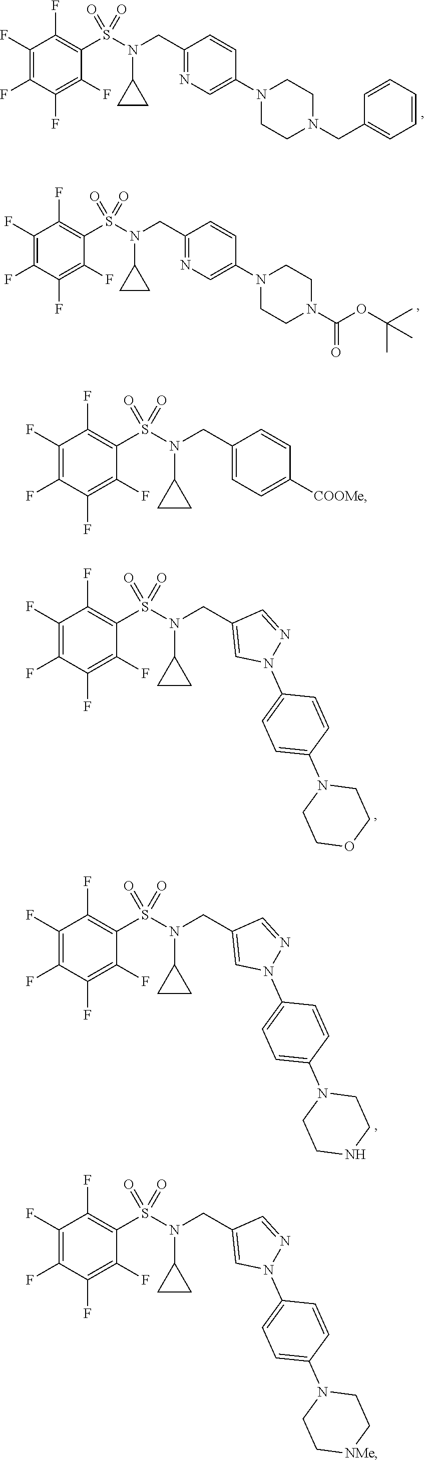

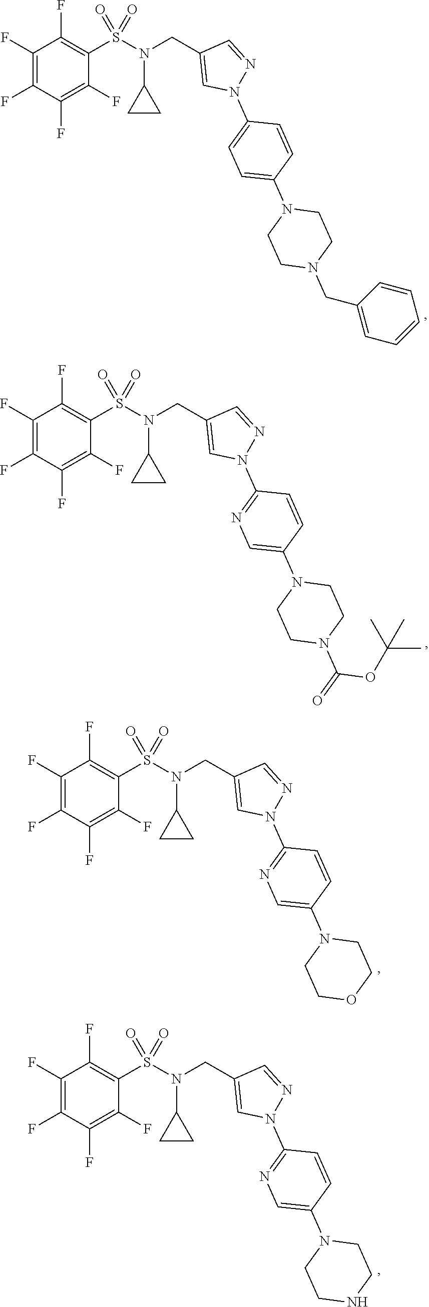 [SMILES: O=S(=O)(c1c(F)c(F)c(F)c(F)c1F)N(Cc1cnn(-c2ccc(N3CCN(Cc4ccccc4)CC3)cc2)c1)C1CC1 ; CC(C)(C)OC(=O)N1CCN(c2ccc(-n3cc(CN(C4CC4)S(=O)(=O)c4c(F)c(F)c(F)c(F)c4F)cn3)nc2)CC1 ; O=S(=O)(c1c(F)c(F)c(F)c(F)c1F)N(Cc1cnn(-c2ccc(N3CCOCC3)cn2)c1)C1CC1 ; O=S(=O)(c1c(F)c(F)c(F)c(F)c1F)N(Cc1cnn(-c2ccc(N3CCNCC3)cn2)c1)C1CC1]

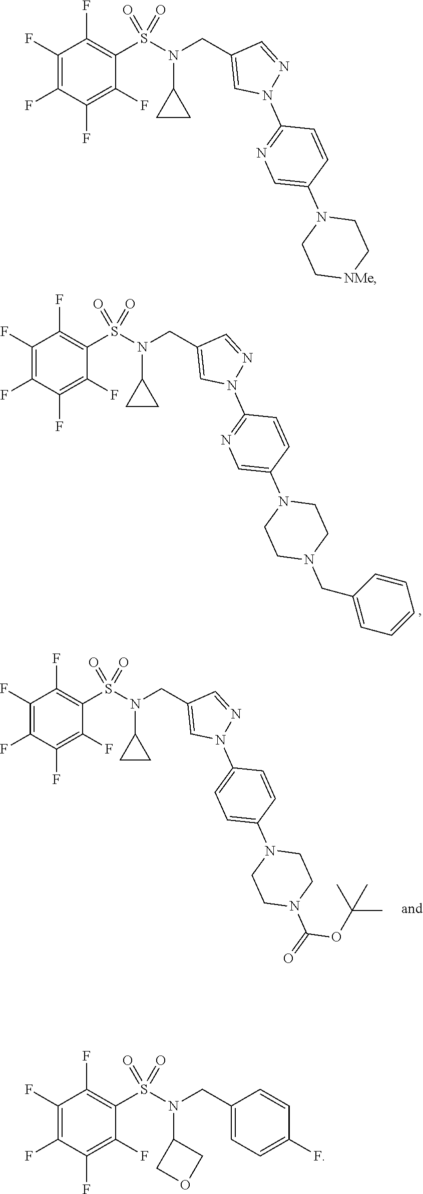 [SMILES: CN1CCN(c2ccc(-n3cc(CN(C4CC4)S(=O)(=O)c4c(F)c(F)c(F)c(F)c4F)cn3)nc2)CC1 ; O=S(=O)(c1c(F)c(F)c(F)c(F)c1F)N(Cc1cnn(-c2ccc(N3CCN(Cc4ccccc4)CC3)cn2)c1)C1CC1 ; CC(C)(C)OC(=O)N1CCN(c2ccc(-n3cc(CN(C4CC4)S(=O)(=O)c4c(F)c(F)c(F)c(F)c4F)cn3)cc2)CC1 ; O=S(=O)(c1c(F)c(F)c(F)c(F)c1F)N(Cc1ccc(F)cc1)C1COC1]

31. The compound of claim 1 selected from:

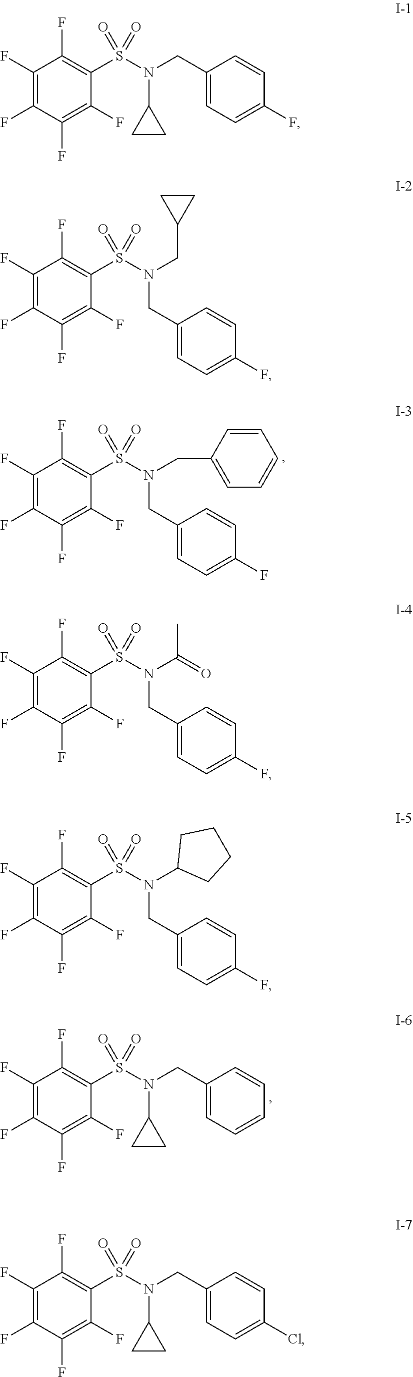

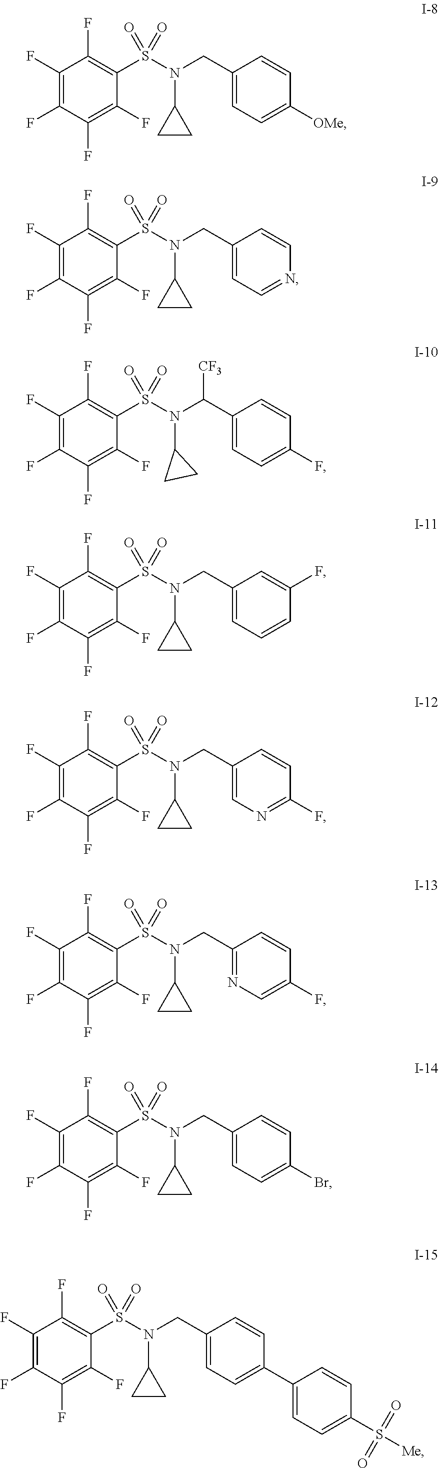

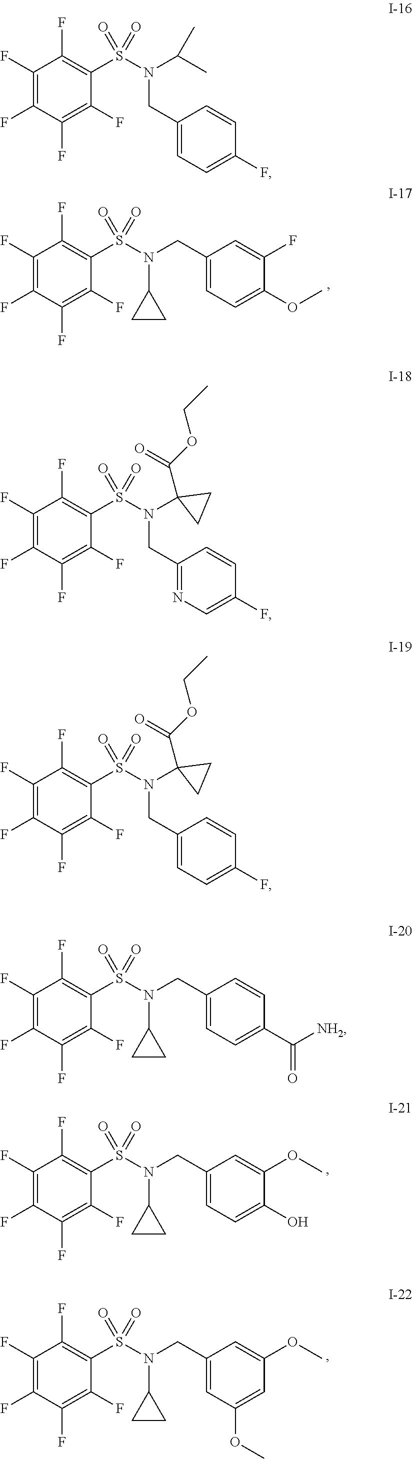

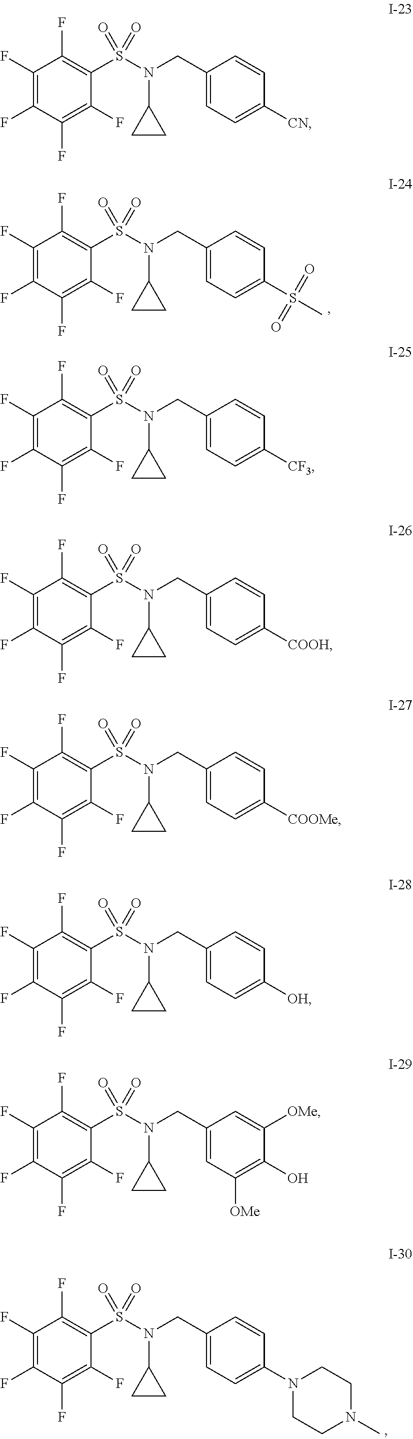

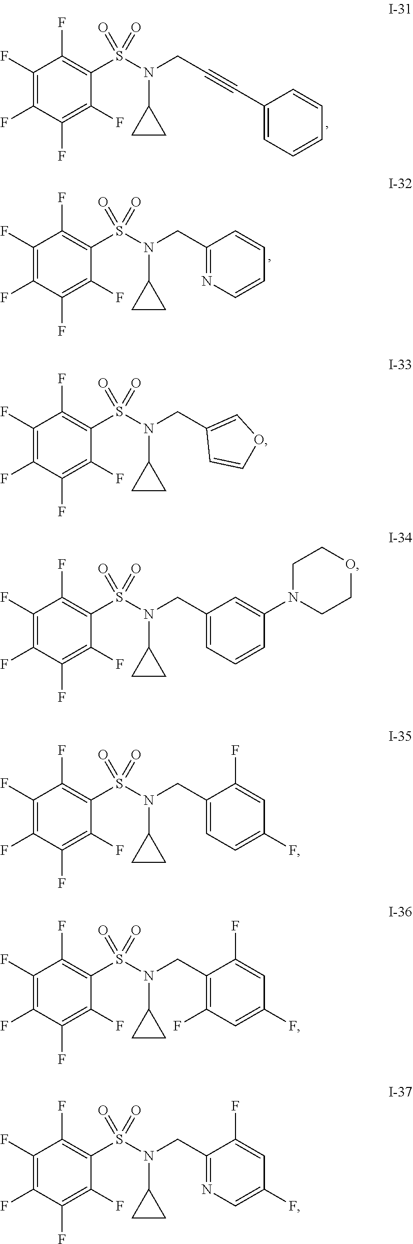

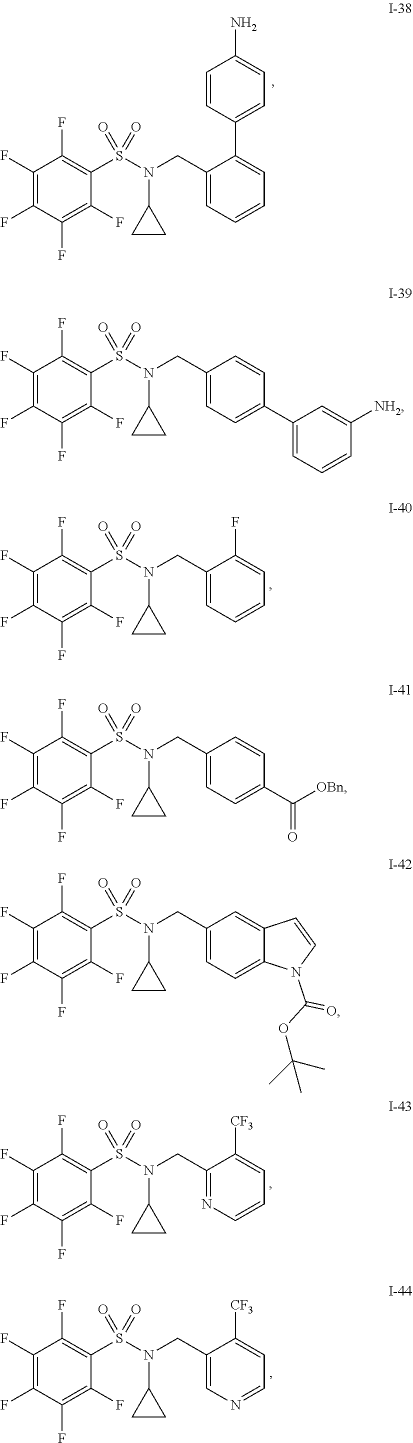

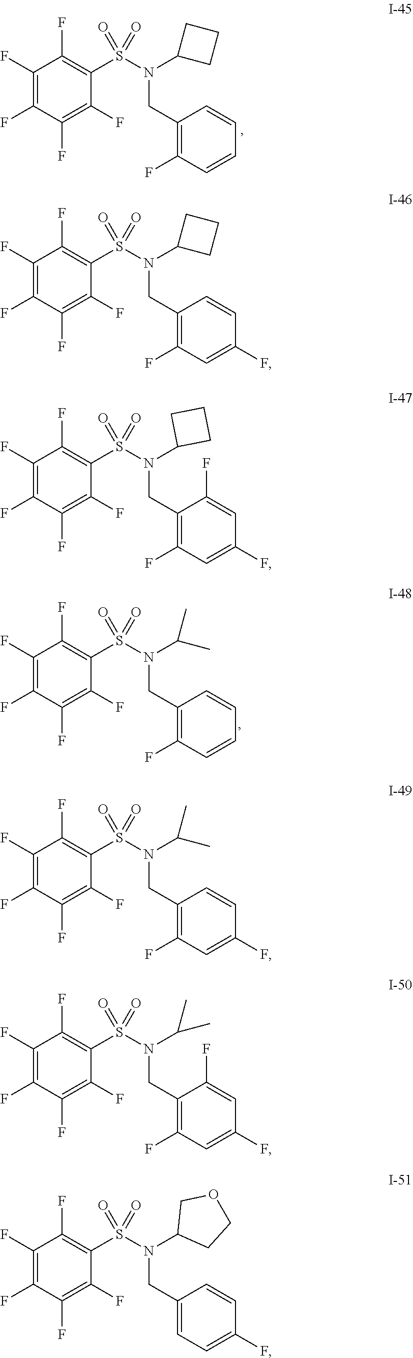

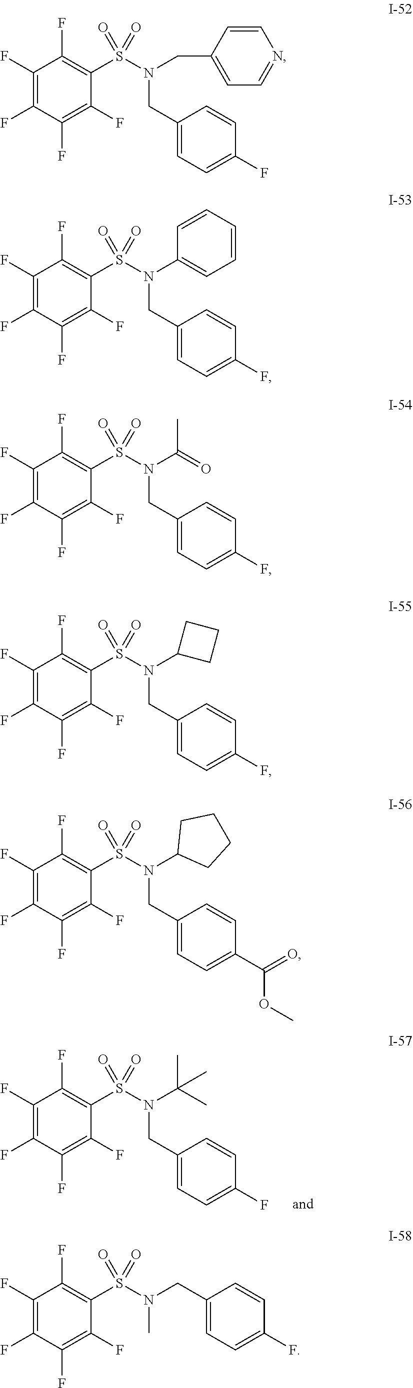

32. A pharmaceutical composition comprising one or more compounds of Formula I of any one of claims 1 to 31 and a pharmaceutically acceptable carrier.

33. A method of treating a cell proliferative disorder comprising administering one or compounds of Formula II, and/or pharmaceutically acceptable salts and/or solvates thereof, to a subject in need thereof: 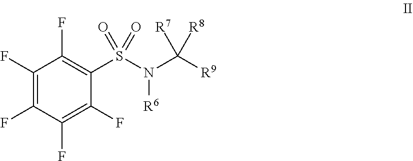
wherein:
R⁶ is selected from C₁₋₁₀alkyl, C₂₋₁₀alkenyl, C₂₋₁₀alkynyl, C(O)C₁₋₁₀alkyl, C₃₋₁₀cycloalkyl, aryl, heterocycloalkyl, heteroaryl, CH₂C₃₋₁₀cycloalkyl, CH₂aryl, CH₂heterocycloalkyl and CH₂heteroaryl, the latter 8 of which are each optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆ alkenyl, OC₂₋₆ alkynyl, C₁₋₆ alkyleneOC₁₋₆ alkyl, C₁₋₆ alkyleneOC₂₋₆ alkenyl, C₁₋₆ alkyleneOC₂₋₆alkynyl, C(O)C₁₋₆ alkyl, C(O)C₂₋₆ alkenyl, C(O)C₂₋₆ alkynyl, C(O)OC₁₋₆ alkyl, C(O)OC₂₋₆ alkenyl, C(O)OC₂₋₆alkynyl, S(O)ₓC₁₋₆alkyl, S(O)ₓC₂₋₆alkenyl, S(O)ₓC₂₋₆alkynyl, C(O)NH₂, C(O)NHC₁₋₆alkyl, C(O)N(C₁₋₆alkyl(C₁₋₆alkyl) and NHC(O)C₁₋₆alkyl;
R⁷, and R⁸ are each independently selected from H, C₁₋₆ alkyl, C₂₋₆ alkenyl and C₂₋₆ alkynyl; or
both R⁷ and R⁸ combine to form ═O, or R⁷ and R⁸ together with the carbon to which they are attached form C₃₋₆cycloalkyl;
R⁹ is selected from aryl, heteroaryl, heterocycloalkyl, C₃₋₁₀cycloalkyl, C≡C-aryl, C≡C-heteroaryl, and C≡C-heterocycloalkyl, each of which is optionally substituted with one or more of halo, CN, OH, NH₂, ═O, CO₂H, SO₂F, C₁₋₁₀alkyl, C₂₋₁₀alkenyl, C₂₋₁₀alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆alkynyl, C(O)OC₁₋₆alkyl, C(O)OC₂₋₆ alkenyl, C(O)OC₂₋₆ alkynyl, S(O)ₓC₁₋₆ alkyl, S(O)ₓC₂₋₆ alkenyl, S(O)ₓC₂₋₆ alkynyl, C(O)NH₂, C(O)NHC₁₋₆ alkyl, C(O)N(C₁₋₆ alkyl(C₁₋₆ alkyl), NHC(O)C₁₋₆ alkyl and R¹⁰;
R¹⁰ is selected from Z—C₃₋₁₀cycloalkyl, Z-heterocycloalkyl, Z-aryl and Z-heteroaryl, each of which is optionally substituted with one or more of halo, CN, OH, NH₂, ═O, CO₂H, SO₂F, C₁₋₁₀alkyl, C₂₋₁₀alkenyl, C₂₋₁₀ alkynyl, NH(C₁₋₆alkyl), N(C₁₋₆alkyl)(C₁₋₆alkyl), OC₁₋₆alkyl, OC₂₋₆alkenyl, OC₂₋₆alkynyl, C₁₋₆alkyleneOC₁₋₆alkyl, C₁₋₆alkyleneOC₂₋₆alkenyl, C₁₋₆alkyleneOC₂₋₆alkynyl, C(O)C₁₋₆alkyl, C(O)C₂₋₆alkenyl, C(O)C₂₋₆ alkynyl, C(O)OC₁₋₆ alkyl, C(O)OC₂₋₆ alkenyl, C(O)OC₂₋₆ alkynyl, S(O)ₓC₁₋₆ alkyl, S(O)ₓC₂₋₆ alkenyl, S(O)ₓC₂₋₆ alkynyl, C(O)NH₂, C(O)NHC₁₋₆ alkyl, C(O)N(C₁₋₆ alkyl(C₁₋₆ alkyl), NHC(O)C₁₋₆alkyl, C₃₋₁₀cycloalkyl, aryl, heteroaryl and heterocycloalkyl, the latter four groups being further optionally substituted by C₁₋₆alkyl, C(O)C₁₋₆alkyl and benzyl;
x is 0, 1 or 2;
Z is selected from a direct bond, C₁₋₄alkylene, O, NH, S, SO and SO₂ and
all alkyl, alkenyl, alkynyl, aryl, heteroaryl, heterocycloalkyl and alkylene groups are optionally halosubstituted.

34. The method of claim 33, wherein the cell proliferative disorder is cancer.

35. The method of claim 34, wherein the cancer is leukemia, bile duct, fibroblast, kidney, mesothelioma, multiple myeloma, liver, central nervous system, soft tissue, pancreas, thyroid, gastric, ovary, upper aerodigestive tract, urinary tract, lung, skin, colorectal, esophagus, breast, uterus, cervix, bone, peripheral nervous system or lymphoma.

36. The method of claim 33, wherein cancer is a hematological cancer or a brain cancer.

37. The method of claim 35, wherein the leukemia is acute myeloid leukemia or acute lymphoblastic leukemia (ALL).

38. The method of claim 36, wherein the brain cancer is glioblastoma or medulloblastoma.

39. A method for inhibiting UFMylation in a cell comprising administering an effective amount of one or more compounds of Formula I according to any one of claims 1 to 31 or one or more compounds of Formula II as defined in claim 33 to the cell.

40. A method of treating a disease, disorder or condition that benefits from inhibiting UFMylation comprising administering an effective amount of one or more compounds of Formula I according to any one of claims 1 to 31 or one or more compounds of Formula II as defined in claim 33 to a subject in need thereof.

41. A method for covalently interacting with ubiquitin-like modifier-activating enzyme 5 (UBA5) in a cell comprising administering an effective amount of one or more compounds of Formula I according to any one of claims 1 to 31 or one or more compounds of Formula II as defined in claim 33 to the cell.

42. A method of treating a disease, disorder or condition that benefits from covalently interacting with UBA5 comprising administering an effective amount of one or more compounds of Formula I according to any one of claims 1 to 31 or one or more compounds of Formula II as defined in claim 33 to a subject in need thereof.

43. The method of claim 42, wherein the disease, disorder or condition that benefits from inhibiting UFMylation is a cancer that is caused by, or has as least as part of its etiology, upregulation of the c-Myc, pS2 and/or cyclin D1 genes.

44. A method of treating a disease, disorder or condition that benefits from covalently interacting with UBA5 comprising administering an effective amount of one or more compounds of the application to a subject in need thereof.

45. The method of claim 44, wherein the disease, disorder or condition that benefits from covalently interacting with UBA5 is a cancer dependent on UBA5 activity.

46. The method of claim 45, wherein the cancer dependent on UBA5 activity is leukemia, bile duct, fibroblast, kidney, mesothelioma, multiple myeloma, liver, central nervous system, soft tissue, pancreas, thyroid, gastric, ovary, upper aerodigestive tract, urinary tract, lung, skin, colorectal, esophagus, breast, uterus, cervix, bone, peripheral nervous system or lymphoma.
